(19) United States
(12) Patent Application Publication
(10) Pub. No.: US 2010/0093691 A1
(43) Pub. Date: Apr. 15, 2010

(54) TOPICAL FORMULATIONS

(75) Inventors: Petra Helga BECK, Charlbury (GB); Marc Barry Brown, Charlbury (GB); Anthony Coates, London (GB); Yanmin Hu, London (GB); Gerlinda Stoddart, Charlbury (GB)

Correspondence Address: SUGHRUE MION, PLLC, 2100 PENNSYLVANIA AVENUE, N.W., SUITE 800, WASHINGTON, DC, 20037, US

(73) Assignee: HELPERBY THERAPEUTICS LIMITED, Shelby, North Yorks (GB)

(21) Appl. No.: 12/513,783

(22) PCT Filed: Nov. 8, 2007
(86) PCT No.: PCT/GB2007/004268

§ 371 Date: Dec. 22, 2009

(30) Foreign Application Priority Data
Nov. 8, 2006 (GB) PCT/GB2006/004178
May 17, 2007 (GB) 0709513.6

(51) Int. Cl. A61K 31/4353

(52) U.S. Cl. 514/210.16

(57) ABSTRACT

There is provided topical pharmaceutical compositions comprising compounds of formula I

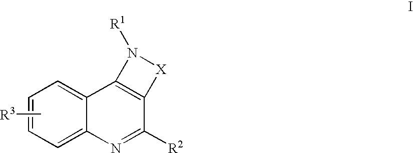

wherein R¹, R², R³ and X have meanings given in the description. These compositions can be used to treat microbial infections and to kill clinically latent microorganisms.

This invention relates to pharmaceutical formulations for topical application comprising compounds based upon the pyrrolo[3,2-c]quinoline ring system. Such formulations may be used to kill microorganisms (including clinically latent microorganisms), and thus have application in the treatment and prophylaxis of certain infections.

The listing or discussion of a prior-published document in this specification should not necessarily be taken as an acknowledgement that the document is part of the state of the art or is common general knowledge.

Before the introduction of antibiotics, patients suffering from acute bacterial infections (e.g. tuberculosis or pneumonia) had a low chance of survival. For example, mortality from tuberculosis was around 50%.

Although the introduction of antibacterial agents in the 1940s and 1950s rapidly changed this picture, bacteria have responded by progressively gaining resistance to commonly used antibiotics. Now, every country in the world has antibiotic-resistant bacteria. Indeed, more than 70% of bacteria that give rise to hospital acquired infections in the USA resist at least one of the main antimicrobial agents that are typically used to fight infection (see Nature Reviews, Drug Discovery 1, 895-910 (2002)).

One way of tackling the growing problem of resistant bacteria is the development of new classes of antimicrobial agents. However, until the introduction of linezolid in 2000, there had been no new class of antibiotic marketed for over 37 years. Moreover, even the development of new classes of antibiotic provides only a temporary solution, and indeed there are already reports of resistance of certain bacteria to linezolid (see Lancet 357, 1179 (2001) and Lancet 358, 207-208 (2001)).

In order to develop more long-term solutions to the problem of bacterial resistance, it is clear that alternative approaches are required. One such alternative approach is to minimise, as much as is possible, the opportunities that bacteria are given for developing resistance to important antibiotics.

Thus, strategies that can be adopted include limiting the use of antibiotics for the treatment of non-acute infections, as well as controlling which antibiotics are fed to animals in order to promote growth.

However, in order to tackle the problem more effectively, it is necessary to gain an understanding of the actual mechanisms by which bacteria generate resistance to antibiotic agents. To do this requires first a consideration of how current antibiotic agents work to kill bacteria.

Antimicrobial agents target essential components of bacterial metabolism. For example, the β-lactams (e.g. penicillins and cephalosporins) inhibit cell wall synthesis, whereas other agents inhibit a diverse range of targets, such as DNA gyrase (quinolones) and protein synthesis (e.g. macrolides, aminoglycosides, tetracyclines and oxazolidinones). The range of organisms against which the antimicrobial agents are effective varies, depending upon which organisms are heavily reliant upon the metabolic step(s) that is/are inhibited. Further, the effect upon bacteria can vary from a mere inhibition of growth (i.e. a bacteriostatic effect, as seen with agents such as the tetracyclines) to full killing (i.e. a bactericidal effect, as seen, for example, with penicillin).

Bacteria have been growing on Earth for more than 3 billion years and, in that time, have needed to respond to vast numbers of environmental stresses. It is therefore perhaps not surprising that bacteria have developed a seemingly inexhaustible variety of mechanisms by which they can respond to the metabolic stresses imposed upon them by antibiotic agents. Indeed, mechanisms by which the bacteria can generate resistance include strategies as diverse as inactivation of the drug, modification of the site of action, modification of the permeability of the cell wall, overproduction of the target enzyme and bypass of the inhibited steps.

Nevertheless, the rate of resistance emerges to a particular agent has been observed to vary widely, depending upon factors such as the agent's mechanism of action, whether the agent's mode of killing is time- or concentration-dependent, the potency against the population of bacteria and the magnitude and duration of the available serum concentration.

It has been proposed (see Science 264, 388-393 (1994)) that agents that target single enzymes (e.g. rifampicin) are the most prone to the development of resistance. Further, the longer that suboptimal levels of antimicrobial agent are in contact with the bacteria, the more likely the emergence of resistance.

Moreover, it is now known that many bacterial infections include sub-populations of bacteria that are phenotypically resistant to antimicrobials (see, for example: J. Antimicrob. Chemother. 4, 395-404 (1988); J. Med. Microbiol. 38, 197-202 (1993); J. Bacteria 182, 1794-1801 (2000); ibid. 182, 6358-6365 (2000); ibid. 183, 6746-6751 (2001); FEMS Microbiol. Lett. 202, 59-65 (2001); and Trends in Microbiology 13, 34-40 (2005)). There appear to be several types of such phenotypically resistant bacteria, including persisters, stationary-phase bacteria, as well as those in the depths of biofilms. However, each of these types is characterised by its low rate of growth (compared to log-phase bacteria under the same conditions). Nutritional starvation and high cell densities are also common characteristics of such bacteria.

Although resistant to antimicrobial agents in their slow-growing state, phenotypically resistant bacteria differ from those that are genotypically resistant in that they regain their susceptibility to antimicrobials when they return to a fast-growing state (e.g. when nutrients become more readily available to them).

The presence of phenotypically resistant bacteria in an infection leads to the need for prolonged courses of antimicrobial agents, comprising multiple doses. This is because the resistant, slowly multiplying bacteria provide a pool of “latent” organisms that can convert to a fast-growing state when the conditions allow (thereby effectively re-initiating the infection). Multiple doses over time deal with this issue by gradually killing off the “latent” bacteria that convert to “active” form.

However, dealing with “latent” bacteria by administering prolonged courses of antimicrobials poses its own problems. That is, prolonged exposure of bacteria to suboptimal concentrations of antimicrobial agent can lead to the emergence of genotypically resistant bacteria, which can then multiply rapidly in the presence of even high concentrations of the antimicrobial.

Long courses of antimicrobials are more likely to encourage the emergence of genotypic resistance than shorter courses on the grounds that non-multiplying bacterial will tend to survive and, interestingly, probably have an enhanced ability to mutate to resistance (see, for example: Proc. Natl. Acad. Sci. USA 92, 11736-11740 (1995); J. Bacteria 179, 6688-6691 (1997); and Antimicrob. Agents Chemother. 44, 1771-1777 (2000)). For example, non-dividing E. coli continually mutates to ciprofloxacin resistance during a seven-day exposure to the agent. Thus, “latent” bacteria might be one of the sources of genotypically resistant bacteria.

In the light of the above, a new approach to combating the problem of bacterial resistance might be to select and develop antimicrobial agents on the basis of their ability to kill “latent” microorganisms. The production of such agents would allow, amongst other things, for the shortening of chemotherapy regimes in the treatment of microbial infections, thus reducing the frequency with which genotypical resistance arises in microorganisms.

Certain pyrrolo[2,3-c]quinolines, as well as their 2,3-dihydro derivatives, are disclosed in: Science of Synthesis 15, 389-549 (2005); Heterocycles 48(2), 221-226 (1998); Tetrahedron 52(2), 647-60 (1996); ibid. 51(47), 12869-82 (1995); Synlett (Spec. Issue), 507-509 (1995); Tetrahedron Lett. 34(22), 3629-32 (1993); JP 48030280; JP 48030078; JP 48030077; Chem. & Pharm. Bull. 20(1), 109-16 (1972); Yakugaku Zasshi 77, 85-9 (1957); ibid. 81, 363-9 (1961); ibid. 81, 479-83 and 484-9 (1961); Acta Crystallographica C43(11), 2206-9 (1987); Acta Chimica Sinica 41(7), 668-71 (1984); ibid. 42(5), 470-8 (1984); J. Chem. Soc., Perkin Trans. 1 1457-63 (1997); and Anti-Cancer Drug Design 9, 51-67 (1994).

Medical utilities of such compounds, for examples as inhibitors of the gastric (H⁺/K⁺)-ATPase, as agents for the treatment of diseases related to corticotropin-releasing factor (CRF) and/or corticotropin-releasing factor receptor, as agents for the prevention and/or treatment of neurodegenerative diseases, as inhibitors of the effects of free radicals, as immunoregulators, as antiinflammatory agents, as analgesics, as antipyretic agents, as hypotensive agents, as inhibitors of enzymes of the kynurenine pathway, as cytotoxic agents, or as inhibitors of HIV particle formation are mentioned in WO 97/44342; WO 98/05660; WO 99/09029; WO 00/01696; WO 01/42247; WO 2005/076861; EP 0 307 078; EP 0 587 473; JP 06092963; U.S. Pat. No. 4,771,052; U.S. Pat. No. 6,995,163; J. Med. Chem. 33(2), 527-33 (1990); Drug Design and Delivery 7,131-8 (1991); J. Med. Chem. 35, 1845-52 (1992); Farmaco 54(3), 152-160 (1999); Bioorg. Med. Chem. Lett. 9, 2819-22 (1999); Biochem. Biophys. Acta 1029, 24-32 (1990); and Eur. J. Med. Chem. 32, 815-22 (1997).

Activity against malaria parasites, Trypanosoma cruzi and amoeba for certain 2,3-dihydropyrrolo[3,2-c]quinoline compounds is mentioned in GB 725 745, U.S. Pat. No. 2,691,023, U.S. Pat. No. 2,691,024 and Synthesis 903-906 (2005).

Further, activity against certain growing bacteria for a small number of 2,3-dihydropyrrolo[3,2-c]quinoline compounds is mentioned in Yakugaku Zasshi 77, 90-3 (1957).

PCT publication no. WO 2007/054693 (application no. PCT/GB2006/004178), a priority document for this application, discloses, inter alia, various pyrrolo[3,2-c]quinolines (and 2,3-dihydro-derivative thereof), as well as the use of such compounds in the killing of clinically latent microorganisms and the treatment of microbial infections.

According to a first aspect of the invention, there is provided a topical pharmaceutical composition comprising a compound of formula I, or a pharmaceutically-acceptable derivative thereof, in admixture with a pharmaceutically acceptable adjuvant, diluent or carrier, wherein the compound of formula I has the following structure,

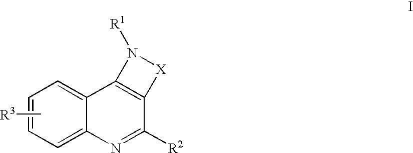

wherein

R¹ represents
(a) H,
(b) C₁₋₁₂ alkyl, C₃₋₁₂ cycloalkyl, C₃₋₁₂ cycloalkenyl (which latter three groups are optionally substituted by one or more substituents selected from halo, nitro, CN, C₁₋₆ alkyl, C₁₋₆ alkenyl, C₁₋₆ alkynyl, C₃₋₈ cycloalkyl (which latter three groups are optionally substituted by one or more substituents selected from OH, ═O, halo, C₁₋₄ alkyl and C₁₋₄ alkoxy), OR^{4a}, S(O)ₙR^{4b}, S(O)₂N (R^{4c})(R^{4d}), N(R^{4e})S(O)₂R^{4f}, N(R^{4g})(R^{4h}), B¹—C(O)—B²—R⁴ⁱ, aryl and Het¹, and which C₃₋₁₂ cycloalkyl or C₄₋₁₂ cycloalkenyl groups may additionally be substituted by ═O),
(c) aryl or
(d) Het²;

R² represents
(a) H,
(b) C₁₋₁₂ alkyl, C₁₋₁₂ alkenyl, C₁₋₁₂ alkynyl, C₃₋₁₂ cycloalkyl or C₄₋₁₂ cycloalkenyl, which latter five groups are optionally substituted by one or more substituents selected from halo, nitro, CN, C₁₋₆ alkyl, C₁₋₆ alkenyl, C₁₋₆ alkynyl, C₃₋₈ cycloalkyl (which latter three groups are optionally substituted by one or more substituents selected from OH, ═O, halo, C₁₋₄ alkyl and C₁₋₄ alkoxy), OR^{5a}, S(O)ₚR^{5b}, S(O)₂N(R^{5c})(R^{5d}), N(R^{5e})S(O)₂R^{5f}, N(R^{5g})(R^{5b}), B³—C(O)—B⁴—R⁵ⁱ, aryl and Het³, and which C₃₋₁₂ cycloalkyl or C₄₋₁₂ cycloalkenyl groups may additionally be substituted by ═O,
(c) aryl or
(d) Het⁴;

R³ represents H or one to four substituents on the fused benzene ring selected from
(a) halo,
(b) CN,
(c) C₁₋₁₂ alkyl, C₁₋₁₂ alkenyl, C₁₋₁₂ alkynyl, C₃₋₁₂ cycloalkyl or C₄₋₁₂ cycloalkenyl, which latter five groups are optionally substituted by one or more substituents selected from halo, nitro, CN, C₁₋₆ alkyl, C₁₋₆ alkenyl, C₁₋₆ alkynyl, C₃₋₈ cycloalkyl (which latter three groups are optionally substituted by one or more substituents selected from OH, ═O, halo, C₁₋₄ alkyl and C₁₋₄ alkoxy), OR^{6a}, S(O)_{q}R^{6b}, S(O)₂N(R^{6c})R^{6d}), N(R^{6e})S(O)₂R^{6f}, N(R^{6g})(R^{6h}), B⁵—C(O)—C⁶—R⁶ⁱ, aryl and Het⁵, and which C₃₋₁₂ cycloalkyl or C₄₋₁₂ cycloalkenyl groups may additionally be substituted by ═O,
(d) OR^{7a},
(e) S(O)ᵣR^{7b},
(f) S(O)₂N(R^{7c})(R^{7d}),
(g) N(R^{7e})S(O)₂R^{7f},
(h) N(R^{7g})(R^{7b}),
(i) B⁷—C(O)—B⁸—R⁷ⁱ,
(j) aryl or
(k) Het⁶;

R^{4a} to R⁴ⁱ, R^{5a} to R⁵ⁱ, R^{6a} to R⁶ⁱ and R^{7a} to R⁷ⁱ independently represent, at each occurrence,
(a) H,
(b) C₁₋₁₀ alkyl, C₂₋₁₀ alkenyl, C₂₋₁₀ alkynyl (which latter three groups are optionally substituted by one or more substituents selected from halo, OH, C₁₋₆ alkoxy, aryl and Het⁷),
(c) C₃₋₁₀ cycloalkyl, C₄₋₁₀ cycloalkenyl (which latter two groups are optionally substituted by one or more substituents selected from halo, OH, ═O, C₁₋₆ alkyl, C₁₋₆ alkoxy, aryl and Het⁸),
(d) aryl or
(e) Het⁹,
   provided that R^{4b}, R^{5b}, R^{6b} or R^{7b} does not represent H when n, p, q or r, respectively is 1 or 2;

X represents
(a) —C(R^{8a})(R^{8b})—C(R^{8c})(R^{8d})— or
(b) —C(R^{8e})═C(R^{8f})—;

R^{8a} to R^{8f} independently represent H, halo or C₁₋₄ alkyl;

each aryl independently represents a C₆₋₁₀ carbocyclic aromatic group, which group may comprise either one or two rings and may be substituted by one or more substituents selected from
(a) halo,
(b) CN,
(c) C₁₋₁₂ alkyl, C₁₋₁₂ alkenyl, C₁₋₁₂ alkynyl, C₃₋₁₂ cycloalkyl or C₄₋₁₂ cycloalkenyl, which latter five groups are optionally substituted by one or more substituents selected from halo, nitro, CN, C₁₋₆ alkyl, C₁₋₆ alkenyl, C₁₋₆ alkynyl, C₃₋₈ cycloalkyl (which latter three groups are optionally substituted by one or more substituents selected from OH, ═O, halo, C₁₋₄ alkyl and C₁₋₄ alkoxy), OR^{9a}, S(O)ₜR^{9b}, S(O)₂N(R^{9e})(R^{9d}), N(R^{9e})S(O)₂R^{9f}, N(R^{9g})(R^{9h}), B⁹—C(O)—B¹⁰—R⁹ⁱ, phenyl, naphthyl (which latter two groups are optionally substituted by one or more substituents selected from OH, halo, C₁₋₄ alkyl and C₁₋₄ alkoxy) and Het¹⁰, and which C₃₋₁₂ cycloalkyl or C₄₋₁₂ cycloalkenyl groups may additionally be substituted by ═O,
(d) OR^{10a},
(e) S(O)ᵤR^{10b},
(f) S(O)₂N(R^{10c})(R^{10d}),
(g) N(R^{10e})S(O)₂R^{10f},
(h) N(R^{10g})(R^{10h}),
(i) B¹¹—C(O)—B¹²—R¹⁰ⁱ,
(j) phenyl (which latter group is optionally substituted by one or more substituents selected from OH, halo, C₁₋₄ alkyl and C₁₋₄ alkoxy) or
(k) Het¹¹;

R^{9a} to R⁹ⁱ and R^{10a} to R¹⁰ⁱ independently represent, at each occurrence,
(a) H,
(b) C₁₋₁₂ alkyl, C₂₋₁₂ alkenyl, C₂₋₁₂ alkynyl, C₃₋₁₂ cycloalkyl, C₄₋₁₂ cycloalkenyl (which latter five groups are optionally substituted by one or more substituents selected from halo, OH, C₁₋₆ alkyl, C₃₋₁₂ cycloalkyl, C₄₋₁₂ cycloalkenyl (which latter two groups are optionally substituted by one or more substituents selected from OH, ═O, halo, C₁₋₄ alkyl and C₁₋₄ alkoxy), C₁₋₆ alkoxy, NH₂, N(H)—C₁₋₆ alkyl, N(C₁₋₆ alkyl)₂, phenyl (which latter group is optionally substituted by one or more substituents selected from OH, halo, C₁₋₄ alkyl and C₁₋₄ alkoxy) and Het¹², and which C₃₋₁₂ cycloalkyl or C₄₋₁₂ cycloalkenyl groups may additionally be substituted by ═O),
(c) phenyl (which latter group is optionally substituted by one or more substituents selected from OH, CN, halo, C₁₋₆ alkyl and C₁₋₆ alkoxy) or
(e) Het¹³,
   provided that R^{9b} or R^{1 0b} does not represent H when t or u, respectively is 1 or 2;

Het¹ to Het¹³ independently represent 4- to 14-membered heterocyclic groups containing one or more heteroatoms selected from oxygen, nitrogen and/or sulfur, which heterocyclic groups may comprise one, two or three rings and may be substituted by one or more substituents selected from
(a) halo,
(b) CN,
(c) C₁₋₁₂ alkyl, C₁₋₁₂ alkenyl, C₁₋₁₂ alkynyl, C₃₋₁₂ cycloalkyl or C₄₋₁₂ cycloalkenyl, which latter five groups are optionally substituted by one or more substituents selected from halo, nitro, CN, C₁₋₆ alkyl, C₁₋₆ alkenyl, C₁₋₆ alkynyl, C₃₋₈ cycloalkyl (which latter three groups are optionally substituted by one or more substituents selected from OH, ═O, halo, C₁₋₄ alkyl and C₁₋₄ alkoxy), OR^{11a}, S(O)ᵥR^{11b}, S(O)₂N(R^{11c})(R^{11d}), N(R^{11e})S(O)₂R^{11f}, N(R^{11g})(R^{11h}), B¹³—C(O)—B¹⁴—R¹¹ⁱ, phenyl, naphthyl (which latter two groups are optionally substituted by one or more substituents selected from OH, halo, C₁₋₄ alkyl and C₁₋₄ alkoxy) and Het^{a}, and which C₃₋₁₂ cycloalkyl or C₄₋₁₂ cycloalkenyl groups may additionally be substituted by ═O,
(d) OR^{12a},
(e) ═O,
(f) S(O)₂R^{12b},
(g) S(O)₂N(R^{12c})(R^{12d}),
(h) N(R^{12e})S(O)₂R^{12f},
(i) N(R^{12g})(R^{12h}),
(j) B¹⁵—C(O)—B¹⁶—R¹²ⁱ,
(k) phenyl (which latter group is optionally substituted by one or more substituents selected from OH, halo, C₁₋₄ alkyl and C₁₋₄ alkoxy) or
(l) Het^{b};

R^{11a} to R¹¹ⁱ and R^{12a} to R¹²ⁱ independently represent, at each occurrence,
(a) H,
(b) C₁₋₁₂ alkyl, C₂₋₁₂ alkenyl, C₂₋₁₂ alkynyl, C₃₋₁₂ cycloalkyl, C₄₋₁₂ cycloalkenyl (which latter five groups are optionally substituted by one or more substituents selected from halo, OH, C₁₋₆ alkyl, C₃₋₁₂ cycloalkyl, C₄₋₁₂ cycloalkenyl (which latter two groups are optionally substituted by one or more substituents selected from OH, ═O, halo, C₁₋₄ alkyl and C₁₋₄ alkoxy), C₁₋₆ alkoxy, phenyl (which latter group is optionally substituted by one or more substituents selected from OH, halo, C₁₋₄ alkyl and C₁₋₄ alkoxy) and Het^{c}, and which C₃₋₁₂ cycloalkyl or C₄₋₁₂ cycloalkenyl groups may additionally be substituted by ═O),
(c) phenyl (which latter group is optionally substituted by one or more substituents selected from OH, halo, C₁₋₄ alkyl and C₁₋₄ alkoxy) or
(e) Het^{d},
   provided that R^{11b} or R^{12b} does not represent H when v or w, respectively is 1 or 2;

B¹ to B¹⁶ independently represent a direct bond, O, S, NH or N(R¹³);

n, p, q, r, s, t, u, v and w independently represent 0, 1 or 2;

R¹³ represents
(a) C₁₋₆ alkyl,
(b) phenyl (which latter group is optionally substituted by one or more substituents selected from OH, halo, C₁₋₄ alkyl and C₁₋₄ alkoxy),
(c) C₃₋₇ cycloalkyl (which latter group is are optionally substituted by one or more substituents selected from OH, ═O, halo, C₁₋₄ alkyl and C₁₋₄ alkoxy) or
(e) Het^{e};

Het^{a} to Het^{e} independently represent 5- or 6-membered heterocyclic groups containing one to four heteroatoms selected from oxygen, nitrogen and/or sulfur, which heterocyclic groups may be substituted by one or more substituents selected from halo, ═O and C₁₋₆ alkyl; and

unless otherwise specified
(i) alkyl, alkenyl, alkynyl, cycloalkyl, and cycloalkenyl groups, as well as the alkyl part of alkoxy groups, may be substituted by one or more halo atoms, and
(ii) cycloalkyl and cycloalkenyl groups may comprise one or two rings and may additionally be ring-fused to one or two benzene rings.

When used herein, the term “pharmaceutically-acceptable derivative” includes references to:
(a) pharmaceutically-acceptable salts with either acids or bases (e.g. acid addition salts); and/or
(b) solvates (e.g. hydrates)

Acid addition salts that may be mentioned include carboxylate salts (e.g. formate, acetate, trifluoroacetate, propionate, isobutyrate, heptanoate, decanoate, caprate, caprylate, stearate, acrylate, caproate, propiolate, ascorbate, citrate, glucuronate, glutamate, glycolate, a-hydroxybutyrate, lactate, tartrate, phenylacetate, mandelate, phenylpropionate, phenylbutyrate, benzoate, chlorobenzoate, methylbenzoate, hydroxybenzoate, methoxybenzoate, dinitrobenzoate, o-acetoxybenzoate, salicylate, nicotinate, isonicotinate, cinnamate, oxalate, malonate, succinate, suberate, sebacate, fumarate, malate, maleate, hydroxymaleate, hippurate, phthalate or terephthalate salts), halide salts (e.g. chloride, bromide or iodide salts), sulfonate salts (e.g. benzenesulfonate, methyl-, bromo- or chloro-benzenesulfonate, xylenesulfonate, methanesulfonate, ethanesulfonate, propanesulfonate, hydroxyethanesulfonate, 1- or 2-naphthalene-sulfonate or 1,5-naphthalenedisulfonate salts) or sulfate, pyrosulfate, bisulfate, sulfite, bisulfate, phosphate, monohydrogenphosphate, dihydrogenphosphate, metaphosphate, pyrophosphate or nitrate salts, and the like.

The term “pharmaceutically-acceptable derivative” also includes references to:
(a) C₁₋₄ alkyl quaternary ammonium salts; or
(b) N-oxides,
   at either of the two tertiary N-atoms of the (2,3-dihydro-)pyrroloquinoline ring system or at a tertiary N-atom that may be present in any of substituents R¹, R² and R³

For the avoidance of doubt, the definitions of the terms aryl, alkyl, alkenyl, alkynyl, cycloalkyl, cycloalkenyl and alkoxy groups provided above apply, unless otherwise stated, at each usage of such terms herein. Further, the one or two benzene rings that may be fused to cycloalkyl groups may bear one or more of the substituents defined in respect of the relevant cycloalkyl group.

The term “halo”, when used herein, includes fluoro, chloro, bromo and iodo.

Heterocyclic (Het¹ to Het¹³ and Het^{a} to Het^{e}) groups may be fully saturated, partly unsaturated, wholly aromatic or partly aromatic in character. Values of heterocyclic (Het¹ to Het¹³ and Het^{a} to Het^{e}) groups that may be mentioned include 1-azabicyclo[2.2.2]octanyl, benzimidazolyl, benzo[c]isoxazolidinyl, benzisoxazolyl, benzodioxanyl, benzodioxepanyl, benzodioxolyl, benzofuranyl, benzofurazanyl, benzomorpholinyl, 2,1,3-benzoxadiazolyl, benzoxazolidinyl, benzoxazolyl, benzopyrazolyl, benzo[e]pyrimidine, 2,1,3-benzothiadiazolyl, benzothiazolyl, benzothienyl, benzotriazolyl, chromanyl, chromenyl, cinnolinyl, 2,3-dihydrobenzimidazolyl, 2,3-dihydrobenzo[b]furanyl, 1,3-dihydrobenzo-[c]furanyl, 1,3-dihydro-2,1-benzisoxazolyl 2,3-dihydropyrrolo[2,3-b]pyridinyl, dioxanyl, furanyl, hexahydropyrimidinyl, hydantoinyl, imidazolyl, imidazo[1,2-a]pyridinyl, imidazo[2,3-b]thiazolyl, indolyl, isoquinolinyl, isoxazolidinyl, isoxazolyl, maleimido, morpholinyl, naphtho[1,2-b]furanyl, oxadiazolyl, 1,2- or 1,3-oxazinanyl, oxazolyl, phthalazinyl, piperazinyl, piperidinyl, purinyl, pyranyl, pyrazinyl, pyrazolyl, pyridinyl, pyrimidinyl, pyrrolidinonyl, pyrrolidinyl, pyrrolinyl, pyrrolo[2,3-b]pyridinyl, pyrrolo[5,1-b)]pyridinyl, pyrrolo[2,3-c]pyridinyl, pyrrolyl, quinazolinyl, quinolinyl, sulfolanyl, 3-sulfolenyl, 4,5,6,7-tetrahydrobenzimidazolyl, 4,5,6,7-tetrahydrobenzopyrazolyl, 5,6,7,8-tetrahydro-benzo[e]pyrimidine, tetrahydrofuranyl, tetrahydropyranyl, 3,4,5,6-tetrahydro-pyridinyl, 1,2,3,4-tetrahydropyrimidinyl, 3,4,5,6-tetrahydropyrimidinyl, thiadiazolyl, thiazolidinyl, thiazolyl, thienyl, thieno[5,1-c]pyridinyl, thiochromanyl, triazolyl, 1,3,4-triazolo[2,3-b]pyrimidinyl, xanthenyl and the like.

Values of Het¹ that may be mentioned include benzodioxanyl (e.g. benzodioxan-2-yl), benzodioxolyl (e.g. benzodioxol-5-yl), pyrazinyl (e.g. pyrazin-2-yl), pyridinyl (e.g. pyridin-2-yl or pyridin-3-yl), pyrrolidinonyl (e.g. pyrrolidinon-1-yl) and tetrahydrofuranyl (e.g. tetrahydrofuran-2-yl).

Values of Het² that may be mentioned include benzimidazolyl (e.g. benzimidazol-2-yl), piperidinyl (e.g. piperidin-4-yl), pyridinyl (e.g. pyridin-3-yl) and pyrrolidinyl (e.g. pyrrolidin-3-yl).

Values of Het⁶ that may be mentioned include morpholinyl (e.g. morpholin-4-yl) and piperidinyl (e.g. piperidin-4-yl).

Values of Het⁹ that may be mentioned include piperidinyl (e.g. piperidin-1-yl).

Values of Het¹¹ that may be mentioned include piperazinyl (e.g. piperazin-1-yl), piperidinyl (e.g. piperidin-1-yl) and pyridinyl (e.g. pyridin-3-yl).

Values of Het¹³ that may be mentioned include pyridinyl (e.g. pyridin-3-yl).

Particular embodiments of the compounds of formula I include those in which:
(1) R¹ represents
   C₁₋₆ alkyl or C₃₋₇ cycloalkyl (which latter two groups are optionally substituted by one or more substituents selected from halo, C₁₋₄ alkyl, C₃₋₆ cycloalkyl (which latter group is optionally substituted by one or more substituents selected from halo, C₁₋₄ alkyl and C₁₋₄ alkoxy), C(O)OR⁴ⁱ, aryl and Het¹),
   aryl or
   Het²;
(2) R² represents C₁₋₆ alkyl optionally substituted by one or more substituents selected from halo, OR^{5a}, N(R^{5g})(R^{5h}) and C(O)OR⁵ⁱ;
(3) R³ represents H or, particularly, one to four substituents on the fused benzene ring selected from
   halo (e.g. chloro),
   CN,
   C₁₋₆ alkyl optionally substituted by one or more substituents selected from halo, CN, and OR^{6a},
   OR^{7a},
   S(O)ᵣR^{7b},
   N(H)R^{7h},
   C(O)R⁷ⁱ,
   C(O)OR⁷ⁱ,
   aryl and
   Het⁶;
(4) R^{4a} to R⁴ⁱ, R^{5a} to R⁵ⁱ, R^{6a} to R⁶ⁱ and R^{7a} to R⁷ⁱ independently represent, at each occurrence,
   C₁₋₁₀ alkyl (optionally substituted by one or more substituents selected from halo and aryl),
   C₃₋₆ cycloalkyl (optionally substituted by one or more substituents selected from halo, C₁₋₄ alkyl and C₁₋₄ alkoxy),
   aryl or
   Het⁹,
   or R^{4a} to R⁴ⁱ, R^{5a} to R⁵ⁱ, R^{6a} to R⁶ⁱ and R^{7c} to R⁷ⁱ may also represent H, provided that R^{4b}, R⁵, R^{6b} or R^{7h} does not represent H when n, p, q or r, respectively is 1 or 2;
(5) X represents —C(H)R^{8a}—C(H)R^{8c}—;
(6) R^{8a} to R^{8f} independently represent H or methyl;
(7) each aryl independently represents a C₆₋₁₀ carbocyclic aromatic group, which group may comprise either one or two rings and may be substituted by one or more substituents selected from
   halo,
   CN,
   C₁₋₆ alkyl optionally substituted by one or more substituents selected from halo, C₃₋₆ cycloalkyl (which latter groups is optionally substituted by one or more substituents selected from halo, C₁₋₄ alkyl and C₁₋₄ alkoxy),
   OR^{9a}, S(O)ₜR^{9b}, S(O)₂N(H)R^{9c}, N(H)S(O)₂R^{9f}, N(R^{9g})(R^{9h}), B⁹—C(O)—B¹⁰—R⁹ⁱ, phenyl (which latter groups is optionally substituted by one or more substituents selected from OH, halo, methyl and methoxy) and Het¹⁰,
   OR^{10a},
   S(O)ᵤR^{10b},
   N(R^{10g})(R^{10h}),
   B¹¹—C(O)—B¹²—R¹⁰ⁱ,
   phenyl (which latter group is optionally substituted by one or more substituents selected from OH, halo, C₁₋₄ alkyl and C₁₋₄ alkoxy) or
   Het“;
(8) R^{9a} to R⁹ⁱ and R^{10a} to R¹⁰ⁱ independently represent, at each occurrence,
   H,
   C₁₋₆ alkyl, C₃₋₆ cycloalkyl (which latter two groups are optionally substituted by one or more substituents selected from halo, OH, C₁₋₄ alkyl, C₄₋₆ cycloalkyl (which latter group is optionally substituted by one or more substituents selected from halo, C₁₋₄ alkyl and C₁₋₄ alkoxy), C₁₋₄ alkoxy, NH₂, N(H)—C₁₋₄ alkyl, N(C₁₋₄ alkyl)₂, phenyl (which latter group is optionally substituted by one or more substituents selected from OH, halo, methyl and methoxy) and Het¹²),
   phenyl (which latter group is optionally substituted by one or more substituents selected from OH, halo, C₁₋₄ alkyl and C₁₋₄alkoxy) or
   Het¹³,
   provided that R^{9b} or R^{10b} does not represent H when t or u, respectively is 1 or 2;
(9) Het¹ to Het¹³ independently represent 5- to 10-membered heterocyclic groups containing from one to four heteroatoms selected from oxygen, nitrogen and/or sulfur, which heterocyclic groups may comprise one or two rings and may be substituted by one or more substituents selected from halo,
   C₁₋₆ alkyl, C₃₋₆ cycloalkyl, which latter two groups are optionally substituted by one or more substituents selected from halo, OH, C₁₋₄ alkyl, C₄₋₆ cycloalkyl (which latter group is optionally substituted by one or more substituents selected from halo, C₁₋₄ alkyl and C₁₋₄ alkoxy), C₁₋₄ alkoxy, phenyl (which latter group is optionally substituted by one or more substituents selected from OH, halo, methyl and methoxy) and Het^{a},
   OR^{12a},
   ═O,
   S(O)_{w}R^{12b},
   N(R^{12g})(R^{12b}),
   B¹⁵—C(O)—B¹⁶—R¹²ⁱ,
   phenyl (which latter group is optionally substituted by one or more substituents selected from OH, halo, methyl and methoxy) or
   Het^{b};
(10) R^{11a} to R¹¹ⁱ and R^{12a} to R¹²ⁱ independently represent, at each occurrence,
   H,
   C₁₋₆ alkyl, C₃₋₆ cycloalkyl (which latter two groups are optionally substituted by one or more substituents selected from halo, OH, C₁₋₄ alkyl, C₄₋₆ cycloalkyl (which latter group is optionally substituted by one or more substituents selected from halo, C₁₋₄ alkyl and C₁₋₄ alkoxy), C₁₋₄ alkoxy, phenyl (which latter group is optionally substituted by one or more substituents selected from OH, halo, methyl and methoxy) and Het^{c},
   phenyl (which latter group is optionally substituted by one or more substituents selected from OH, halo, methyl and methoxy) or
   Het^{d},
   provided that R^{11b} or R^{12b} does not represent H when v or w, respectively is 1 or 2;
(11) B¹ to B¹⁶ independently represent a direct bond, O, S or NH;
(12) R¹³ represents C₁₋₄ alkyl or phenyl (which latter group is optionally substituted by one or more substituents selected from OH, halo, methyl and methoxy);
(13) Het^{a} to Het^{e} independently represent 5- or 6-membered heterocyclic groups containing one or heteroatoms selected from oxygen, nitrogen and/or sulfur, which heterocyclic groups may be substituted by one or more substituents selected from halo, ═O and methyl;
(14) unless otherwise specified, alkyl, alkenyl, alkynyl, cycloalkyl, and cycloalkenyl groups, as well as the alkyl part of alkoxy groups, are unsubstituted;
(15) unless otherwise specified, cycloalkyl groups comprise one or (if sufficient number of C-atoms is present) two rings and are optionally ring-fused to a benzene ring (so as to form a group such as, for example, 1,2,3,4-tetrahydronaphthyl or, particularly, indanyl).

More particular embodiments of the compounds of formula I include those in which:
(1) R¹ represents
   C₁₋₅ alkyl (which latter group is optionally substituted by one or more substituents selected from fluoro, C₃₋₅ cycloalkyl (which latter group is optionally substituted by one or more substituents selected from fluoro, methyl and methoxy), C₁₋₄ alkoxy (e.g. methoxy), phenoxy, phenyl (which latter group is optionally substituted by one or more substituents selected from halo, C₁₋₄ alkyl and C₁₋₄ alkoxy) and Het¹),
   C₃₋₆ cycloalkyl, (which latter group is optionally fused to a benzene ring (e.g. to form a group such as indanyl or 1,2,3,4-tetrahydronaphthyl) and is optionally substituted by one or more substituents selected from fluoro, methyl and methoxy),
   phenyl (which latter group is optionally substituted by one or more substituents selected from halo, C₁₋₆ alkyl (which latter group is optionally substituted by one or more substituents selected from OR^{9a}, N(R^{9g})(R^{9h}) and phenyl), OR^{10a} and Het¹¹), or
   Het²;
(2) Het¹ represents 5- to 10-membered, aromatic or part-aromatic heterocyclic group containing from one to four heteroatoms selected from oxygen, nitrogen and/or sulfur, which heterocyclic group comprises either one or two rings and is optionally substituted by one or more substituents selected from halo, C₁₋₃ alkyl (e.g. methyl) and C₁₋₃ alkoxy (e.g. methoxy) (e.g. Het¹ represents a 9-or 10-membered, aromatic or part-aromatic heterocyclic group containing one or two heteroatoms selected from oxygen and nitrogen, such as a benzodioxanyl or benzodioxolyl group);
(3) Het² represents a 5- to 10-membered, heterocyclic group containing from one to four heteroatoms selected from oxygen, nitrogen and/or sulfur, which heterocyclic group comprises either one or two rings and is optionally substituted by one or more substituents selected from halo, C₁₋₃ alkyl (which latter group is optionally substituted by phenyl) and OR^{12a} (e.g. Het² represents a 5- or 6-membered, aromatic or fully saturated heterocyclic group containing one or two heteroatoms selected from oxygen and nitrogen, such as a pyridyl or piperidinyl group, which group is optionally substituted by C₁₋₂ alkyl (which latter group is optionally substituted by phenyl), C₁₋₃ alkoxy (e.g. methoxy) or phenoxy);
(4) Het¹¹ represents a 5- or 6-membered, fully saturated, partly unsaturated or aromatic heterocyclic group containing one or two heteroatoms selected from oxygen, nitrogen and/or sulfur, which heterocyclic group is optionally substituted by one or more substituents selected from halo and C₁₋₃ alkyl (e.g. Het¹¹ represents a 6-membered, fully saturated heterocyclic group containing one or two heteroatoms selected from oxygen and nitrogen, such as a piperazinyl group, which group is optionally substituted by C₁₋₃ alkyl (e.g. methyl));

(⁵) R^{9a} to R⁹ⁱ independently represent, at each occurrence, H or C₁₋₃ alkyl (e.g. methyl);
(6) R^{10a} represents, independently at each occurrence,
   H,
   C₁₋₄ alkyl, C₅₋₆ cycloalkyl (which latter two groups are optionally substituted by one or more substituents selected from halo, methyl, methoxy, NH₂, N(H)CH₃, N(CH₃)₂ or phenyl),
   phenyl (which latter group is optionally substituted by one or more substituents selected from halo, methyl and methoxy) or
   Het¹³;
(7) Het¹³ represents a 5- to 10-membered, aromatic heterocyclic group containing from one to four heteroatoms selected from oxygen, nitrogen and/or sulfur, which heterocyclic group comprises either one or two rings and is optionally substituted by one or more substituents selected from halo, C₁₋₃ alkyl (e.g. methyl) and C₁₋₃ alkoxy (e.g. methoxy) (e.g. Het² represents a 5- or 6-membered, aromatic heterocyclic group containing one or two heteroatoms selected from oxygen and nitrogen, such as an unsubstituted pyridyl group);
(7) R₁₂ represents C₁₋₆ alkyl, C₅₋₆ cycloalkyl (which latter two groups are optionally substituted by one or more substituents selected from halo, methyl and methoxy) or phenyl (which latter group is optionally substituted by one or more substituents selected from halo, methyl and methoxy);
(8) R² represents C₁₋₃ alkyl optionally substituted by one or more substituents selected from halo, OH and N(H)R^{5g} (e.g. R² represents unsubstituted C₁₋₃ alkyl, such as methyl);
(9) R³ represents one to three (e.g. one or two) substituents on the fused benzene ring selected from
   C₁₋₃ alkyl (optionally substituted by one or more halo groups),
   N(H)R^{7h},
   Het⁶ or, particularly,
   OR^{7a};
(10) R^{7a} represents, independently at each occurrence,
   C₁₋₆ alkyl (optionally substituted by one or more substituents selected from halo and phenyl (which latter group is optionally substituted by one or more substituents selected from halo, C₁₋₄ alkyl and C₁₋₄ alkoxy)),
   C₅₋₆ cycloalkyl (optionally substituted by one or more substituents selected from halo, methyl and methoxy),
   phenyl (which latter group is optionally substituted by one or more substituents selected from halo, C₁₋₄ alkyl and C₁₋₄ alkoxy) or

Het⁹;
(11) R^{7a} represents phenyl optionally substituted by one or more substituents selected from halo, C₁₋₄ alkyl and C₁₋₄ alkoxy);
(12) Het⁶ represents a 5- or 6-membered fully saturated heterocyclic group containing one or two heteroatoms selected from oxygen and nitrogen (e.g. a piperidinyl (such as a piperidin-1-yl) group), which group is optionally substituted by one or more C₁₋₂ alkyl groups;
(13) Het⁹ represents a 5- to 10-membered, aromatic heterocyclic group containing from one to four heteroatoms selected from oxygen, nitrogen and/or sulfur, which heterocyclic group comprises either one or two rings and is optionally substituted by one or more substituents selected from halo, C₁₋₃ alkyl (e.g. methyl) and C₁₋₃ alkoxy (e.g. methoxy);
(14) X represents —CH₂—CH₂—.

Certain particular embodiments of the compound of formula I include those in which the compound may be represented as a compound of formula Ia,

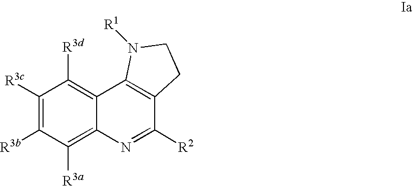

wherein R¹ and R² are as hereinbefore defined and each of R^{3a} to R^{3d} represents either H or a substituent as hereinbefore defined in relation to the group R³.

Hereinafter, references to compounds of formula I are, unless the context indicates otherwise, intended to include references to compounds of formula Ia. Conversely, where reference is made to particular embodiments of the compounds of formula Ia, these embodiments apply equally, where relevant, to compounds of formula I.

Particular embodiments of the compounds of formula Ia that may be mentioned include those in which:
(1) one or both of R^{3a} and R^{3c} represent a substituent as hereinbefore defined in relation to the group R³, and R^{3b} and R^{3d} both represent H;
(2) R^{3a} and R^{3c} independently represent H, OR^{7a}, N(H)R^{7h} or Het⁶ (e.g. H or OR^{7a}), wherein R^{7a}, R^{7h} and Het⁶ are as hereinbefore defined, provided that R^{3a} and R^{3c} do not both represent H.

More particular embodiments of the compounds of formula Ia that may be mentioned include those in which:

R^{l} represents
C₁₋₅ alkyl (which latter group is optionally substituted by C₃₋₅ cycloalkyl (e.g. cyclopropyl), phenyl (which latter group is optionally substituted by one or more substituents selected from halo, methyl and methoxy), phenoxy, benzodioxanyl (e.g. benzodioxan-2-yl) or benzodioxolyl (e.g. benzodioxol-5-yl),
C₃₋₆ cycloalkyl (which latter group is optionally fused to a benzene ring (e.g. to provide a cycloalkyl group such as cyclopropyl, or a benzo-fused cycloalkyl group such as 1,2,3,4-tetrahydronaphthyl or indanyl (e.g. indan-2-yl, indan-1-yl, (S)-indan-1-yl or (R)-indan-1-yl))),
phenyl (which latter group is optionally substituted by one or more substituents (e.g. one or two substituents, such as a single substituent in the 4-position) selected from halo (e.g. fluoro), C₁₋₄ alkyl (e.g. methyl or, particularly, iso-propyl), OH, C₁₋₄ alkoxy (which latter group is optionally substituted by N(CH₃)₂, providing, for example, a methoxy or OCH₂CH₂N(CH₃)₂ group), phenoxy (which latter group is either unsubstituted or is substituted by one or more, e.g. one or two, substituents selected from methoxy or, particularly, halo (such as fluoro)), piperidin-1-yl, pyridyloxy (e.g. pyrid-3-yloxy) and piperazinyl (optionally substituted by methyl, providing, for example, 4-methylpiperazin-1-yl) (e.g. one or more substituents selected from those listed above, other than piperidin-1-yl),
pyridyl (e.g. pyrid-3-yl), which latter group is optionally substituted (e.g. in the 6-position) by methoxy or phenoxy, or
piperidinyl (e.g. piperidin-4-yl), which latter group is optionally substituted (e.g. at the 1-position) by C₁₋₂ alkyl (which latter group is optionally substituted by phenyl, providing, for example, benzyl);

R^{3a} and R^{3c} independently represent
H,
C₁₋₄ alkoxy (optionally substituted by one or more halo atoms (e.g. to provide a substituted alkoxy group such as trifluoromethoxy or, particularly, an unsubstituted alkoxy group such as methoxy or ethoxy)),
—N(H)-(phenyl), the phenyl part of which latter group is (optionally substituted by one or more substituents selected from halo, methyl and methoxy),
a 5- or 6-membered N-linked, fully saturated heterocycle containing an N-atom (that via which the group is linked) and optionally containing one further heteroatom selected from N and O (e.g. a piperidin-1-yl group) or
phenoxy (optionally substituted by one or more substituents selected from halo, methyl and methoxy),
(e.g. R^{3a} and R^{3c} independently represent H, C₁₋₄ alkoxy (optionally substituted by one or more halo atoms (e.g. to provide a substituted alkoxy group such as trifluoromethoxy or, particularly, an unsubstituted alkoxy group such as methoxy or ethoxy)), or phenoxy (optionally substituted by one or more substituents selected from halo, methyl and methoxy)),
   provided that R^{3a} and R^{3c} do not both represent H.

Further, in compounds of formula Ia, embodiments of the group R^{l} that may be mentioned include phenyl substituted (e.g. at the 4-position) by a C₃₋₁₂ alkyl group (e.g. a branched C₃₋₁₂ alkyl group, such as iso-propyl), and optionally further substituted as defined above in respect of R¹ (when that group represents aryl).

Specific embodiments of the compounds of formula Ia that may be mentioned further include those in which:
(1) R^{3a} and R^{3c} are both other than H (e.g. R^{3a} and R^{3c} both represent OR^{7a}, wherein R^{7a} is as hereinbefore defined), and R^{3b} and R^{3d} both represent H;
(2) R^{3a} is other than H (e.g. R^{3a} represents OR^{7a}, wherein R^{7a} is as hereinbefore defined), and R^{3b}, R^{3c} and R^{3d} all represent H; or, particularly,
(3) R^{3c} is other than H (e.g. R^{3c} represents OR^{7a}, wherein R^{7a} is as hereinbefore defined), and R^{3a}, R^{3b} and R^{3d} all represent H.

Specific values of R¹ that may be mentioned in relation to compounds of formula I include 3-methylbut-1-yl, 1-methylbenzimidazol-2-yl, cyclopropyl, cyclopropylmethyl, 2-phenoxyethyl, benzodioxol-5-ylmethyl, 6-methoxypyridin-3-yl, 6-phenoxypyridin-3-yl, 3-hydroxyphenyl, 3-hydroxy-5-methylphenyl, 4-hydoxyphenyl, 4-(2-dimethylaminoethoxy)phenyl, 3-fluoro-4-(4-methylpiperazin-1-yl)phenyl, 4-(pyridin-3-yloxy)phenyl or, particularly, benzodioxan-2-ylmethyl, 1-benzylpiperidin-4-yl, cyclohexyl, 1,2,3,4-tetrahydronaphth-1-yl, 1-phenylethyl, 2-phenylethyl, phenyl, 4-iso-propylphenyl, 4-methoxyphenyl, 3-phenoxyphenyl, 4-phenoxyphenyl, benzyl, (2-methylphenyl)methyl, indan-1-yl or indan-2-yl.

Other specific values of R¹ that may be mentioned in relation to compounds of formula I include 3-methoxypropyl, ethoxycarbonylmethyl, 2-(methoxycarbonyl)ethyl, 2-(ethoxycarbonyl)ethyl, 3-(methoxycarbonyl)propyl, 3-(ethoxycarbonyl)propyl, 1-benzylpyrrolidin-3-yl, 1-methylpiperidin-4-yl, tetrahydrofuran-2-ylmethyl, 2-pyridylmethyl, 5-methylpyrazin-2-ylmethyl, 2-(2-pyridyl)ethyl, 2-(3-pyridyl)ethyl, 3-(1-pyrrolidin-2-onyl)propyl, 2-methylphenyl, 4-(piperidin-1-yl)phenyl, 4-(3-pyridyl)phenyl, 2-phenylpropyl or, particularly, (S)-indan-1-yl, (R)-indan-1-yl, 2-(4-chlorophenyl)ethyl, 2-(4-methoxyphenyl)ethyl or 4-(4-fluorophenoxy)phenyl.

Particular compounds of formula Ia that may be mentioned include those of formula Ib,

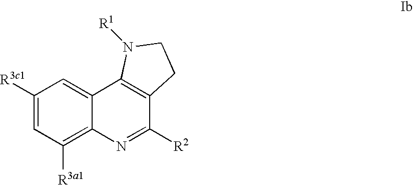

wherein:

R¹ and R² are as hereinbefore defined;

R^{3a1} represents H and R^{3c1} represents phenoxy,

or, when R¹ represents
C₁₋₂ alkyl substituted by optionally substituted phenyl (e.g. benzyl, (2-methylphenyl)methyl, 1-phenylethyl or, particularly, 2-phenylethyl),
C₅₋₆ cycloalkyl fused to a benzene ring (e.g. 1,2,3,4-tetrahydronaphthyl, indan-1-yl or, particularly, indan-2-yl), or
phenyl substituted by phenoxy or piperidin-1-yl,
   then R^{3a1} can additionally represent methoxy, or phenoxy and R^{3c1} can additionally represent H, piperidin-1-yl, methoxy, trifluoromethoxy or ethoxy, provided that R^{3a1} and R^{3c1} do not both represent phenoxy.

Hereinafter, references to compounds of formula I (or Ia) are, unless the context indicates otherwise, intended to include references to compounds of formula Ib. Conversely, where reference is made to particular embodiments of the compounds of formula Ib, these embodiments apply equally, where relevant, to compounds of formula I (or Ia).

Embodiments of the compounds of formula Ib include those in which:
(1) R¹ represents
   (a) C₁₋₅ alkyl, which latter group is optionally substituted by C₃₋₅ cycloalkyl, phenyl (which latter group is optionally substituted by one or more substituents selected from halo, methyl and methoxy), phenoxy, benzodioxanyl or benzodioxolyl,
   (b) C₃₋₆ cycloalkyl, which latter group is optionally fused to a benzene ring,
   (c) phenyl, which latter group is optionally substituted by one or more substituents selected from halo, C₁₋₄ alkyl, OH, C₁₋₄ alkoxy (which latter group is optionally substituted by N(CH₃)₂), phenoxy (which latter group is optionally substituted by one or more substituents selected from methoxy and halo), piperidin-1-yl, pyridyloxy and piperazinyl (which latter group is optionally substituted by methyl),
   (d) pyridyl, which latter group is optionally substituted by methoxy or phenoxy, or
   (e) piperidinyl, which latter group is optionally substituted by C₁₋₂ alkyl (which latter group is optionally substituted by phenyl); and
(2) R² represents C₁₋₃ alkyl optionally substituted by one or more halo substituents.

Other embodiments of the compounds of formula Ib that may be mentioned include those in which:
(1) R^{l} represents
   (a) C₁₋₅ alkyl (e.g. C₁₋₄ alkyl, such as C₁₋₃ alkyl or, particularly, C₁₋₂ alkyl), which latter group is either unsubstituted or, in a particular embodiment, is substituted by cyclopropyl, phenyl (which latter group is optionally substituted by one or more substituents selected from halo, methyl and methoxy), phenoxy, benzodioxan-2-yl or benzodioxol-5-yl,
   (b) cyclopropyl, 1,2,3,4-tetrahydronaphthyl or indanyl (e.g. indan-2-yl, indan-1-yl, (S)-indan-1-yl or (R)-indan-1-yl),
   (c) phenyl, which latter group is optionally substituted by one or two substituents (such as a single substituent in the 4-position) selected from fluoro, methyl, iso-propyl, OH, methoxy or OCH₂CH₂N(CH₃)₂, phenoxy (which latter group is either unsubstituted or is substituted by one or two substituents selected from methoxy or, particularly, fluoro), piperidin-1-yl, pyrid-3-yloxy and 4-methylpiperazin-1-yl,
   (d) pyridin-3-yl, which latter group is optionally substituted (e.g. in the 6-position) by methoxy or phenoxy, or
   (e) piperidin-4-yl, which latter group is optionally substituted (e.g. at the 1-position) by C₁₋₂ alkyl (which latter group is optionally substituted by phenyl, providing, for example, benzyl);
(2) R² represents methyl;
(3) R^{3a} represents H and R^{3b} represents phenoxy, or, when R¹ represents
   benzyl, (2-methylphenyl)methyl, 1-phenylethyl or 2-phenylethyl,
   1,2,3,4-tetrahydronaphthyl, indan-1-yl or indan-2-yl, or
   phenyl substituted by phenoxy or piperidin-1-yl,
   then R^{3a} can additionally represent methoxy or phenoxy and R^{3b} can additionally represent H, piperidin-1-yl, methoxy, trifluoromethoxy or ethoxy,
   provided that R^{3a1} and R^{3c1} do not both represent phenoxy.

Specific compounds of formula I, Ia and Ib that may be mentioned include the following compounds:
(a) 6,8-dimethoxy-1-(4-iso-propylphenyl)-4-methyl-2,3-dihydro-1H-pyrrolo[3,2-c]quinoline;
(b) 6-methoxy-1-(4-phenoxyphenyl)-4-methyl-2,3-dihydro-1H-pyrrolo[3,2-c]quinoline;
(c) 6-methoxy-1-(4-iso-propylphenyl)-4-methyl-2,3-dihydro-1H-pyrrolo[3,2-c]quinoline;
(d) 6,8-dimethoxy-1-(4-phenoxyphenyl)-4-methyl-2,3-dihydro-1H-pyrrolo[3,2-c]quinoline;
(e) 4-methyl-8-phenoxy-1-(4-phenoxyphenyl)-2,3-dihydro-1H-pyrrolo[3,2-c]quinoline;
(f) 1-(4-iso-propylphenyl)-6-phenoxy-4-methyl-2,3-dihydro-1H-pyrrolo[3,2-c]quinoline; and
(g) 4,6-dimethyl-1-(4-methylphenyl)-2,3-dihydro-1H-pyrrolo[3,2-c]quinoline,
   as well as, particularly, the compounds of Preparative Examples 1 to 8 below.

When used herein, the term “compounds of Preparative Examples 1 to 8 below” refers to the title compounds of those examples, i.e.:
(1) 6,8-dimethoxy-4-methyl-1-(3-phenoxyphenyl)-2,3-dihydro-1H-pyrrolo[3,2-c]-quinoline;
(2) 6,8-dimethoxy-4-methyl-1-(2-phenoxyethyl)-2,3-dihydro-1H-pyrrolo[3,2-c]-quinoline;
(3) 1-cyclopropyl-6,8-dimethoxy-4-methyl-2,3-dihydro-1H-pyrrolo[3,2-c]-quinoline;
(4) 8-methoxy-4-methyl-1-(4-phenoxyphenyl)-2,3-dihydro-1H-pyrrolo[3,2-c]-quinoline;
(5) {2-[4-(8-methoxy-4-methyl-2,3-dihydro-1H-pyrrolo[3,2-c]quinolin-1-yl)-phenyoxy]ethyl}dimethylamine;
(6) 8-methoxy-4-methyl-1-[4-(pyridin-3-yloxy)phenyl]-2,3-dihydro-1H-pyrrolo[3,2-c]quinoline;
(7) 4-methyl-8-phenoxy-1-phenyl-2,3-dihydro-1H-pyrrolo[3,2-c]quinoline;
(8) 1-benzyl-4-methyl-8-phenoxy-2,3-dihydro-1H-pyrrolo[3,2-c]quinoline
(9) 1-(indan-2-yl)-4-methyl-8-phenoxy-2,3-dihydro-1H-pyrrolo[3,2-c]quinoline
(10) 4-methyl-6-phenoxy-1-phenyl-2,3-dihydro-1H-pyrrolo[3,2-c]quinoline;
(11) 1-benzyl-4-methyl-6-phenoxy-2,3-dihydro-1H-pyrrolo[3,2-c]quinoline;
(12) 1-(indan-2-yl)-4-methyl-6-phenoxy-2,3-dihydro-1H-pyrrolo[3,2-c]quinoline;
(13) 4-methyl-1-(2-phenylethyl)-8-phenoxy-2,3-dihydro-1H-pyrrolo[3,2-c]-quinoline;
(14) 8-methoxy-4-methyl-1-(2-phenylethyl)-2,3-dihydro-1H-pyrrolo[3,2-c]-quinolin-6-ol;
(15) 1-(1-benzyl-piperidin-4-yl)-4-methyl-8-phenoxy-2,3-dihydro-1H-pyrrolo[3,2-c]quinoline;
(16) 1-(indan-1-yl)-4-methyl-8-phenoxy-2,3-dihydro-1H-pyrrolo[3,2-c]quinoline;
(17) 1-(benzodioxan-2-ylmethyl)-4-methyl-8-phenoxy-2,3-dihydro-1H-pyrrolo[3,2-c]quinoline;
(18) 4-methyl-8-phenoxy-1-(1,2,3,4-tetrahydronaphthalen-1-yl)-2,3-dihydro-1H-pyrrolo[3,2-c]quinoline;
(19) 1-cyclohexyl-4-methyl-8-phenoxy-2,3-dihydro-1H-pyrrolo[3,2-c]quinoline;
(20) 8-ethoxy-4-methyl-1-(4-phenoxyphenyl)-2,3-dihydro-1H-pyrrolo[3,2-c]-quinoline;
(21) 1-(4-methoxyphenyl)-4-methyl-8-phenoxy-2,3-dihydro-1H-pyrrolo[3,2-c]-quinoline;
(22) 4-methyl-1-(4-phenoxyphenyl)-2,3-dihydro-1H-pyrrolo[3,2-c]quinoline; (23) 4-methyl-1-(2-methylphenyl)methyl-8-phenoxy-2,3-dihydro-1H-pyrrolo[3,2-c]quinoline;
(24) 4-methyl-8-phenoxy-1-(4-iso-propylphenyl)-2,3-dihydro-1H-pyrrolo[3,2-c]-quinoline;
(25) 4-methyl-8-phenoxy-1-(1-phenylethyl)-2,3-dihydro-1H-pyrrolo[3,2-c]-quinoline;
(26) 8-methoxy-4-methyl-1-(2-phenylethyl)-2,3-dihydro-1H-pyrrolo[3,2-c]-quinoline;
(27) 6,8-dimethoxy-1-(4-hydroxyphenyl)-4-methyl-2,3-dihydro-1H-pyrrolo[3,2-c]quinoline;
(28) 6,8-dimethoxy-1-(3-hydroxyphenyl)-4-methyl-2,3-dihydro-1H-pyrrolo[3,2-c]quinoline;

(29) 6,8-dimethoxy-1-(3-hydroxy-5-methylphenyl)-4-methyl-2,3-dihydro-1H-pyrrolo[3,2-c]quinoline;
(30) 8-methoxy-1-(4-methoxyphenyl)-4-methyl-2,3-dihydro-1H-pyrrolo[3,2-c]-quinoline;
(31) 8-trifluoromethoxy-1-(4-phenoxyphenyl)-4-methyl-2,3-dihydro-1H-pyrrolo[3,2-c]quinoline;
(32) 6,8-dimethoxy-4-methyl-1-[4-(pyridin-3-yloxy)phenyl]-2,3-dihydro-1H-pyrrolo[3,2-c]quinoline;
(33) 1-benzyl-6,8-dimethoxy-4-methyl-2,3-dihydro-1H-pyrrolo[3,2-c]quinoline;
(34) 6,8-dimethoxy-4-methyl-1-(2-phenylethyl)-2,3-dihydro-1H-pyrrolo[3,2-c]quinoline;
(35) 4-methyl-1-(2-phenylethyl)-8-trifluoromethoxy-2,3-dihydro-1H-pyrrolo[3,2-c]quinoline;
(36) 6,8-dimethoxy-1-(indan-1-yl)-4-methyl-2,3-dihydro-1H-pyrrolo[3,2-c]-quinoline;
(37) 6,8-dimethoxy-4-methyl-1-[(6-phenoxy)pyridin-3-yl]-2,3-dihydro-1H-pyrrolo[3,2-c]quinoline;
(38) 6,8-dimethoxy-1-[(6-methoxy)pyridin-3-yl]-4-methyl-2,3-dihydro-1H-pyrrolo[3,2-c]quinoline;
(39) 1-(benzodioxol-5-ylmethyl)-6,8-dimethoxy-4-methyl-2,3-dihydro-1H-pyrrolo[3,2-c]quinoline;
(40) 6,8-dimethoxy-4-methyl-1-(3-methylbutyl)-2,3-dihydro-1H-pyrrolo[3,2-c]-quinoline;
(41) 1-cyclopropylmethyl-6,8-dimethoxy-4-methyl-2,3-dihydro-1H-pyrrolo[3,2-c]quinoline;
(42) 4-methyl-8-(morpholin-4-yl)-1-(4-phenoxyphenyl)-2,3-dihydro-1H-pyrrolo[3,2-c]quinoline;
(43) 8-methoxy-4-methyl-1-(1,2,3,4-tetrahydronaphthalen-1-yl)-2,3-dihydro-1H-pyrrolo[3,2-c]quinoline;
(44) 4-methyl-1-(2-phenylethyl)-2,3-dihydro-1H-pyrrolo[3,2-c]quinoline;
(45) 4,6-dimethyl-1-(2-methylphenyl)-2,3-dihydro-1H-pyrrolo[3,2-c]quinoline;
(46) 4,6-dimethyl-1-(2-phenylethyl)-2,3-dihydro-1H-pyrrolo[3,2-c]quinoline;
(47) 4-methyl-8-(piperidin-1-yl)-1-[4-(piperidin-1-yl)phenyl]-2,3-dihydro-1H-pyrrolo[3,2-c]quinoline;
(48) 4-methyl-8-(piperidin-1-yl)-1-(3-phenoxyphenyl)-2,3-dihydro-1H-pyrrolo[3,2-c]quinoline;
(49) 1-{4-[2-(N,N-dimethylamino)ethoxy]phenyl}-4-methyl-8-phenoxy-2,3-dihydro-1H-pyrrolo[3,2-c]quinoline;
(50) 1-[4-(4-fluorophenoxy)phenyl]-8-methoxy-4-methyl-2,3-dihydro-1H-pyrrolo[3,2-c]quinoline;
(51) 1-(benzodioxan-2-ylmethyl)-8-methoxy-4-methyl-2,3-dihydro-1H-pyrrolo[3,2-c]quinoline;
(52) 1-cyclohexyl-8-methoxy-4-methyl-2,3-dihydro-1H-pyrrolo[3,2-c]quinoline;
(53) 8-methoxy-4-methyl-1-phenyl-2,3-dihydro-1H-pyrrolo[3,2-c]quinoline;
(54) 4-methyl-8-phenoxy-1-[4-(3-pyridyl)phenyl]-2,3-dihydro-1H-pyrrolo[3,2]-quinoline;
(55) 4-methyl-8-phenoxy-1-[2-(3-pyridyl)ethyl]-2,3-dihydro-1H-pyrrolo[3,2-quinoline;
(56) 4-methyl-8-phenoxy-1-(2-pyridylmethyl)-2,3-dihydro-1H-pyrrolo[3,2-c]-quinoline;
(57) 4-methyl-1-(5-methylpyrazin-2-ylmethyl)-8-phenoxy-2,3-dihydro-1H-pyrrolo[3,2-c]quinoline;
(58) 8-chloro-4-methyl-1-(2-phenylethyl)-2,3-dihydro-1H-pyrrolo[3,2-c]-quinoline;
(59) methyl 4-methyl-1-(2-phenylethyl)-2,3-dihydro-1H-pyrrolo[3,2-c]quinoline-8-carboxylate;
(60) 4-methyl-8-(morpholin-1-yl)-1-(2-phenylethyl)-2,3-dihydro-1H-pyrrolo[3,2-c]quinoline;
(61) ethyl [4-methyl-1-(2-phenylethyl)-2,3-dihydro-1H-pyrrolo[3,2-c]quinoline-8-yl]acetate;
(62) 1-[3-(4-methyl-8-phenoxy-2,3-dihydro-1H-pyrrolo[3,2-c]quinolin-1-yl)propyl]-pyrrolidin-2-one;
(63) 4-methyl-8-phenoxy-1-[2-(2-pyridyl)ethyl]-2,3-dihydro-1H-pyrrolo[3,2-c]quinoline;
(64) ethyl 3-(8-methoxy-4-methyl-2,3-dihydro-1H-pyrrolo[3,2-c]quinoline-1-yl)propionate;
(65) ethyl 4-(4-methyl-8-phenoxy-2,3-dihydro-1H-pyrrolo[3,2-c]quinoline-1-yl)butanoate;
(66) methyl 4-(4-methyl-8-phenoxy-2,3-dihydro-1H-pyrrolo[3,2-c]quinoline-1-yl)butanoate;
(67) ethyl (4-methyl-8-phenoxy-2,3-dihydro-1H-pyrrolo[3,2-c]quinoline-1-yl)acetate;
(68) 4-methyl-1-(1-methylpiperidin-4-yl)-8-phenoxy-2,3-dihydro-1H-pyrrolo[3,2-c]quinoline;
(69) 1-(1-benzylpyrrolidin-3-yl)-8-methoxy-4-methyl-2,3-dihydro-1H-pyrrolo[3,2-c]quinoline;
(70) methyl 3-(4-methyl-8-phenoxy-2,3-dihydro-1H-pyrrolo[3,2-c]quinoline-1-yl)propionate;
(71) 1-((S)-indan-1-yl)-4-methyl-8-phenoxy-2,3-dihydro-1H-pyrrolo[3,2-c]-quinoline;
(72) 1-((R)-indan-1-yl)-4-methyl-8-phenoxy-2,3-dihydro-1H-pyrrolo[3,2-c]-quinoline;
(73) 1-(3-methoxypropyl)-4-methyl-8-phenoxy-2,3-dihydro-1H-pyrrolo[3,2-c]-quinoline;
(74) 4-methyl-8-phenoxy-1-(tetrahydrofuran-2-ylmethyl)-2,3-dihydro-1H-pyrrolo[3,2-c]quinoline;
(75) 1-[2-(4-chlorophenyl)ethyl]-4-methyl-8-phenoxy-2,3-dihydro-1H-pyrrolo[3,2-c]quinoline;
(76) 1-[2-(4-methoxyphenyl)ethyl]-4-methyl-8-phenoxy-2,3-dihydro-1H-pyrrolo[3,2-c]quinoline;
(77) 4-methyl-8-phenoxy-1-(2-phenylpropyl)-2,3-dihydro-1H-pyrrolo[3,2-c]-quinoline;
(78) 8-cyano-4-methyl-1-(2-phenylethyl)-2,3-dihydro-1H-pyrrolo[3,2-c]quinoline;
(79) 8-hydroxy-4-methyl-1-(2-phenylethyl)-2,3-dihydro-1H-pyrrolo[3,2-c]-quinoline;
(80) 8-phenoxy-1-(2-phenylethyl)-2,3-dihydro-1H-pyrrolo[3,2-c]quinoline;
(81) 6,8-dimethoxy-1-(4-hydroxyphenyl)-4-methylpyrrolo[3,2-c]quinoline;
(82) 8-methoxy-4-methyl-1-[4-(4-methylpiperazin-1-yl)-3-fluorophenyl]-2,3-dihydro-1H-pyrrolo[3,2-c]quinoline;
(83) 4-methyl-8-phenylamino-1-(2-phenylethyl)-2,3-dihydro-1H-pyrrolo[3,2-c]-quinoline; (84) [4-methyl-1-(2-phenylethyl)-2,3-dihydro-1H-pyrrolo[2,3-c]quinoline-8-oyl]-piperidine,
   and pharmaceutically-acceptable salts and/or solvates thereof.

Embodiments of the compound of formula I include those in which:
(1) R¹ represents cyclohexyl or, particularly, 2-phenylethyl;
(2) R² represents methyl;
(3) R^{3a} represents H;
(4) R^{3b} represents phenoxy.

More particular embodiments of the compound of formula I include those in which the compound is:
8-methoxy-4-methyl-1-(4-phenoxyphenyl)-2,3-dihydro-1H-pyrrolo[3,2-c]-quinoline;
4-methyl-8-phenoxy-1-(4-iso-propylphenyl)-2,3-dihydro-1H-pyrrolo[3,2-c]-quinoline;
1-(indan-2-yl)-4-methyl-8-phenoxy-2,3-dihydro -1H-pyrrolo[3,2-c]quinoline;
1-benzyl-4-methyl-8-phenoxy-2,3-dihydro-1H-pyrrolo[3,2-c]quinoline;
4-methyl-8-phenoxy-1-phenyl-2,3-dihydro-1H-pyrrolo[3,2-c]quinoline;
1-(benzodioxan-2-ylmethyl)-4-methyl-8-phenoxy-2,3-dihydro-1H-pyrrolo[3,2-c]quinoline; p0 4-methyl-8-phenoxy-1-(1,2,3,4-tetrahydronaphthalen-1-yl)-2,3-dihydro-1H-pyrrolo[3,2-c]quinoline;
1-cyclohexyl-4-methyl-8-phenoxy-2,3-dihydro-1H-pyrrolo[3,2-c]quinoline;
8-ethoxy-4-methyl-1-(4-phenoxyphenyl)-2,3-dihydro-1H-pyrrolo[3,2-c]-quinoline;
4-methyl-8-(piperidin-1-yl)-1-[4-(piperidin-1-yl)phenyl]-2,3-dihydro-1H-pyrrolo[3,2-c]quinoline;
4-methyl-8-(piperidin-1-yl)-1-(3-phenoxyphenyl)-2,3-dihydro-1H-pyrrolo[3,2-c]quinoline;
1-(indan-1-yl)-4-methyl-8-phenoxy-2,3-dihydro -1H-pyrrolo[3,2-c]quinoline;
1-((S)-indan-1-yl)-4-methyl-8-phenoxy-2,3-dihydro-1H-pyrrolo[3,2-c]-quinoline;
1-((R)-indan-1-yl)-4-methyl-8-phenoxy-2,3-dihydro-1H-pyrrolo[3,2-c]-quinoline;
1-[2-(4-chlorophenyl)ethyl]-4-methyl-8-phenoxy-2,3-dihydro-1H-pyrrolo[3,2-c]quinoline;
1-[2-(4-methoxyphenyl)ethyl]-4-methyl-8-phenoxy-2,3-dihydro-1H-pyrrolo[3,2-c]quinoline;
4-methyl-8-phenoxy-1-(2-phenylpropyl)-2,3-dihydro-1H-pyrrolo[3,2-c]-quinoline;
   or, particularly,
4-methyl-1-(2-phenyl ethyl)-8-phenoxy-2,3-dihydro-1H-pyrrolo[3,2-c]quinoline,
   or a pharmaceutically-acceptable salt and/or solvate thereof (e.g. a hydrochloride salt thereof).

In addition to the above, compounds of formula I that may be mentioned include the following.
(1) A compound of formula I, as hereinbefore defined, provided that the compound is not of the formula

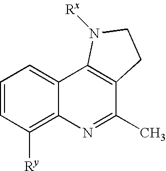

in which:
(a) R^{y} represents H and R^{x} represents H, methyl, 2-hydroxyethyl, phenyl, 4-methylphenyl, 4-methoxyphenyl or 2-chlorophenyl;
(b) R^{y} represents methoxy and R^{x} represents phenyl; or
(c) R^{y} represents hydroxy and R^{x} represents methyl, 2-hydroxyethyl or phenyl.
(In other words, the compound is not:
4-methyl-2,3-dihydro-1H-pyrrolo[3,2-c]quinoline;
1,4-dimethyl-2,3-dihydro-1H-pyrrolo[3,2-c]quinoline;

1-[2-hydroxyethyl]-4-methyl-2,3-dihydro-1H-pyrrolo[3,2-c]quinoline;
4-methyl-1-phenyl-2,3-dihydro-1H-pyrrolo[3,2-c]quinoline;
4-methyl-1-(4-methylphenyl)-2,3-dihydro-1H-pyrrolo[3,2-c]quinoline;
4-methyl-1-(4-methoxyphenyl)-2,3-dihydro-1H-pyrrolo[3,2-c]quinoline;
4-methyl-1-(2-chlorophenyl)-2,3-dihydro-1H-pyrrolo[3,2-c]quinoline;

6-methoxy-4-methyl-1-phenyl-2,3-dihydro-1H-pyrrolo[3,2-c]quinoline;
6-hydroxy-1,4-dimethyl-2,3-dihydro-1H-pyrrolo[3,2-c]quinoline;
6-hydroxy-1-[2-hydroxyethyl]-4-methyl-2,3-dihydro-1H-pyrrolo[3,2-c]quinoline; or
6-hydroxy-4-methyl-1-phenyl-2,3-dihydro-1H-pyrrolo[3,2-c]quinoline.)
   (2) A compound of formula I, as hereinbefore defined, provided that R³ does not represent H or a single OR^{7a} substituent at the 6-position of the pyrrolo[3,2-c]quinoline or 2,3-dihydro-1H-pyrrolo[3,2-c]quinoline ring system, wherein R^{7a} either:
      (a) is as hereinbefore defined;
      (b) represents H or C₁₋₁₀ alkyl; or
      (c) represents H or methyl.
   (3) A compound of formula I, as hereinbefore defined, in which X represents —C(R^{8a})(R^{8b})—C(R^{8c})(R^{8d})—, wherein R^{8a} to R^{8d} are as hereinbefore defined.
   (4) A compounds of formula I, as hereinbefore defined, except that the compound is not:
      (a) of the following formula

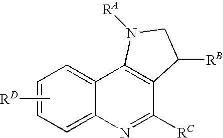

in which
(i) R^{A} represents methyl, benzyl or CH₂CH₂N(C₁₋₂ alkyl)₂,
   R^{B} represents H,
   R^{C} represents H or methyl and
   R^{D} represents H or one or two substituents selected from Cl, OH,
   C₁₋₂ alkoxy and C(0)N(H)CH₃,
(ii) R^{A} represents CH(C₂H₅)₂ or CH(C₂H₅)(CH₂OCH₃),
   R^{B} represents H,
   R^{C} represents methyl or 2,4,6-trimethylphenyl and
   R^{D} represents a single substituent selected from 2,4,6-trimethylphenyl and iodo,
(iii) R^{A} represents benzyl, 1-phenylethyl or phenyl, which latter group is substituted at the 2-position by methyl or methoxy and is optionally further substituted at the 4-position by fluoro, OH, methyl, methoxy or benzyloxy, or at the 6-position by methyl,
   R^{B} represents H, C₁₋₃ alkyl (e.g. methyl, ethyl or iso-propyl) or C₁₋₂ alkyl terminated by OH,
   R^{C} represents H, methyl or hydroxymethyl and
   R^{D} represents H or a single substituent (e.g. at the 6-position) selected from F, OH, methyl, methoxy, trifluoromethoxy, OCH₂CH₂OH or OCH₂CF₃,
(iv) R^{A} represents methyl, 2-hydroxyethyl or phenyl, which latter group is optionally singly substituted in the 2-position by chloro or in the 4-position by methyl or methoxy,
   R^{B} represents H,
   R^{C} represents methyl and
   R^{D} represents H or a single substituent (e.g. at the 6-position) selected from OH and methoxy,
(v) R^{A} represents phenyl substituted by a single OH or methoxy group,
   R^{B} represents H,
   R^{C} represents methyl and
   R^{D} represents H or
(vi) R^{A} represents H or phenyl optionally substituted by a single substituent (e.g. at the 4-position) selected from methyl, chloro or fluoro, or by a single trifluoromethyl substituent (e.g. at the 3-position),
   R^{B} represents H,
   R^{C} represents methyl and
   R^{D} represents a single chloro or fluoro substituent (e.g. at the 8-position) or two substituents (e.g. at the 6- and 8- or 6- and 9-positions) which are both either chloro or methoxy; or
(b) of the following formula

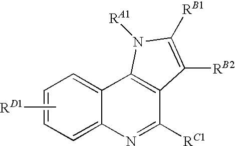

in which
(i) R^{A1} represents 2-ethoxyethyl or CH(R^{alk1})(R^{alk2}), wherein R^{alk1} and
   R^{alk2} independently represent ethyl, n-propyl or methoxymethyl,
   R^{B1} and R^{B2} both represent H,
   R^{C1} represents methyl or 2,4,6-trimethylphenyl and
   R^{D1} represents a single substituent (e.g. at the 6- or 7-position) that is selected from iodo, methyl, aryl or Het⁶, wherein aryl and Het⁶ are as hereinbefore defined, or R^{D1} represents a methyl substituent at the 6-position and a mesityl substituent at the 7-position,
(ii) R^{A1} represents C₁₋₂ alkyl, 1-phenylethyl or phenyl, which latter group is substituted at the 2-position by methyl or methoxy and is optionally further substituted at the 4-position by F, OH, methoxy, acetoxy or benzyloxy,
   R^{B1} represents H,
   R^{B2} represents H, C₁₋₃ alkyl or C₁₋₂ alkyl terminated by OH,
   R^{C1} represents H or methyl and
   R^{D1} represents a single substituent (e.g. at the 6-position) that is selected from Cl, OH, methoxy, trifluoromethoxy, OCH₂CH₂OH or OCH₂CF₃,
(iii) R^{A1} represents methyl, n-butyl, benzyl or phenyl, which latter group is substituted at the 2-position by methyl and is optionally further substituted at the 4-position by F, methoxy, OC(O)O-i-butyl or OC(O)-i-butyl,
   R^{B1} represents H, methyl, hydroxymethyl, n-propyl or phenyl,
   R^{B2} represents H, C₁₋₃ alkyl, hydroxymethyl or phenyl,
   R^{C1} represents H or methyl and
   R^{D1} represents a single substituent (e.g. at the 6-position) that is selected from Cl and methoxy,
(iv) R^{A1} represents phenyl, which group is optionally substituted by F or methoxy,
   R^{B1}, R^{B2} and R^{C1} all represent trifluoromethyl and
   R^{D1} represents H or a single substituent that is selected from F and methoxy,
(v) R^{A1}, R^{B1}, R^{B2} and R^{C1} all represent methyl and
   R^{D1} represents one or two substituents selected from Cl, methyl and methoxy,
(vi) R^{A1} represents methyl, ethyl, 2-ethoxyethyl, 2-isopropoxyethyl, 3-methoxypropyl, n-butyl or phenyl,
   R^{B1} represents methyl, hydroxymethyl or n-propyl,
   R^{B2} represents H or phenyl,
   R^{C1} represents H or methyl and
   R^{D1} represents a single substituent (e.g. at the 6-position) that is selected from Cl, methoxy and 2,4,6-trimethylphenyl or
(vii) R^{A1} represents phenyl,
   R^{B1} and R^{B2} both represent H,
   R^{C1} represents methyl and

R^{D1} represents H or a single methoxy substituent (e.g. at the 8-position).
(5) A compound of formula I, as defined in (4) immediately above, except that the compound is not:
   (a) of the following formula

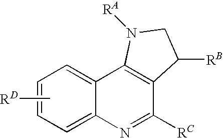

in which
(i) R^{A} represents 2-(dimethylamino)ethyl,
   R^{B} represents H,
   R^{C} represents methyl and
   R^{D} represents one or two substituents selected from Cl, OH and methoxy,
(ii) R^{A} represents phenyl substituted by one or two substituents consisting of an ethyl group at the 4-position or one or two methoxy groups at the 2- and/or 4-positions,
   R^{B} represents H,
   R^{C} represents methyl and

R^{D} represents one or two substituents, at the 6- and/or 8-positions, selected from trifluoromethyl and methoxy or
(iii) R^{A} represents methyl or phenyl, which latter group is optionally substituted by a single substituent selected from Cl, F, methyl, trifluoromethyl and methoxy, or by two methyl groups (e.g. at the 2- and 6-positions),
   R^{B} represents H,
   R^{C} represents H or methyl and
   R^{D} represents H or one or two substituents selected from Cl, F, methyl and methoxy; or
(b) (i) 7,9-dibromo-6-hydroxy-4-methyl-1-phenyl-2,3-dihydro-1H-pyrrolo[3,2-c]quinoline,
(ii) 6-methoxy-4,5-dimethyl-1-(2-methylphenyl)-2,3-dihydro-1H-pyrrolo[3,2-c]quinolinium iodide;
(iii) 1-ethyl-5-methyl-2,3-dihydro-1H-pyrrolo[3,2-c]quinolinium hexafluorophosphate;
(iv) 6,8-dimethoxy-1-(4-iso-propylphenyl)-4-methyl-2,3-dihydro-1H-pyrrolo[3,2-c]quinoline,
(v) 6-methoxy-1-(4-phenoxyphenyl)-4-methyl-2,3-dihydro-1H-pyrrolo[3,2-c]quinoline,
(vi) 6-methoxy-1-(4-iso-propylphenyl)-4-methyl-2,3-dihydro-1H-pyrrolo[3,2-c]quinoline,
(vii) 6,8-dimethoxy-1-(4-phenoxyphenyl)-4-methyl-2,3-dihydro-1H-pyrrolo[3,2-c]quinoline,
(viii) 6,8-dimethoxy-1-(4-hydroxyphenyl)-4-methyl-2,3-dihydro-1H-pyrrolo[3,2-c]quinoline,
(ix) 6,9-dimethoxy-1,2,3,4-tetramethyl-1H-pyrrolo[3,2-c]quinoline,
(x) 6,9-dimethoxy-1,2,3,4,8-pentamethyl-1H-pyrrolo[3,2-c]quinoline,
(xi) 2,3-difluoro-1-phenyl-2,3,4-tris(trifluoromethyl)-2,3-dihydro-1H-pyrrolo[3,2-c]quinoline,
(xii) 6,9-dimethoxy-2,3,4,8-tetramethyl-2,3-dihydro-1H-pyrrolo[3,2-c]quinoline,
(xiii) 7-chloro-3-methyl-2,3-dihydro-1H-pyrrolo[3,2-c]quinoline,
(xiv) 8-chloro-3-methyl-2,3-dihydro-1H-pyrrolo[3,2-c]quinoline,
(xv) 7-fluoro-3-methyl-2,3-dihydro-1H-pyrrolo[3,2-c]quinoline,
(xvi) 7-chloro-3-phenyl-2,3-dihydro-1H-pyrrolo[3,2-c]quinoline or
(xvii) 4-phenyl-2,3-dihydro-1H-pyrrolo[3,2-c]quinoline.
(6) A compound of formula I, as defined in (4) immediately above, except that the compound is not:
   (xviii) 1-(4-methoxyphenyl)-4-methyl-8-phenoxy-2,3-dihydro-1H-pyrrolo[3,2-c]quinoline;
   (xix) 4-methyl-8-phenoxy-1-(4-phenoxyphenyl)-2,3-dihydro-1H-pyrrolo[3,2-c]quinoline;
   (xx) 1-(4-iso-propylphenyl)-8-phenoxy-4-methyl-2,3-dihydro-1H-pyrrolo[3,2-c]quinoline;
   (xxi) 1-(4-iso-propylphenyl)-6-phenoxy-4-methyl-2,3-dihydro-1H -pyrrolo[3,2-c]quinoline;
   (xxii) 6-phenoxy-1-(4-phenoxyphenyl)-4-methyl-2,3-dihydro-1H-pyrrolo[3,2-c]quinoline; or
   (xxiii) 6-phenoxy-1-(4-trifluoromethoxyphenyl)-4-methyl-2,3-dihydro-1H-pyrrolo[3,2-c]quinoline.
(7) A compounds of formula I, as hereinbefore defined, except that the compound is not:
   1-(4-methoxyphenyl)-4-methyl-8-phenoxy-2,3-dihydro-1H-pyrrolo[3,2-c]quinoline;
   4-methyl-8-phenoxy-1-(4-iso-propylphenyl)-2,3-dihydro-1H-pyrrolo[3,2-c]quinoline; or
   6,8-dimethoxy-1-(4-hydroxyphenyl)-4-methyl-2,3-dihydro-1H-pyrrolo[3,2-c]quinoline.

Particular compounds of formulae I that may be mentioned include those in which:
(a) R¹ is other than H;
(b) R² is other than H; and
(c) R¹ and R² are both other than H.

Other particular compounds of formula I that may be mentioned include those in which:
(1) X represents —CH₂—CH₂—;
(2) R¹ represents
   (a) C₁₋₁₂ alkenyl, C₁₋₁₂ alkynyl, C₃₋₁₂ cycloalkyl or C₄₋₁₂ cycloalkenyl, which latter four groups are optionally substituted by one or more substituents selected from halo, nitro, CN, C₁₋₆ alkyl, C₁₋₆ alkenyl, C₁₋₆ alkynyl, C₃₋₈ cycloalkyl (which latter three groups are optionally substituted by one or more substituents selected from OH, ═O, halo, C₁₋₄ alkyl and C₁₋₄ alkoxy), OR^{5a}, S(O)ₚR^{5b}, S(O)₂N(R^{5c})(R^{5d}), N(R^{5e})S(O)₂R^{5f}, N(R^{5g})(R^{5h}), B³—C(O)—B⁴—R⁵ⁱ, aryl and Het³, and which C₃₋₁₂ cycloalkyl or C₄₋₁₂ cycloalkenyl groups may additionally be substituted by ═O,
   (b) aryl substituted as defined above in respect of aryl groups, except that the substituents include at least one OR^{10a} group in which R^{10a} is other than H or C₁₋₁₂ alkyl (which latter group is optionally substituted as defined above in respect of R^{10a}) or
   (c) Het²;
(3) R¹ represents
   (a) C₃₋₁₂ cycloalkyl, which latter groups is optionally fused to a benzene ring and is optionally substituted by one or more substituents selected from halo, nitro, CN, C₁₋₆ alkyl, C₁₋₆ alkenyl, C₁₋₆ alkynyl, C₃₋₈ cycloalkyl (which latter three groups are optionally substituted by one or more substituents selected from OH, ═O, halo, C₁₋₄ alkyl and C₁₋₄ alkoxy), OR^{5a}, ═O, S(O)ₚR^{5b}, S(O)₂N(R^{5c})(R^{5d}), N(R^{5e})S(O)₂R^{5f}, N(R^{5g})(R^{5h}), B³—C(O)—B⁴—R⁵ⁱ, aryl and Het³,
   (b) phenyl substituted as defined above in respect of aryl groups, except that the substituents include at least one OR^{10a} group in which R^{10a} is phenyl (which latter group is optionally substituted by one or more substituents selected from OH, halo, methyl and methoxy) or
   (c) Het²;
(4) R¹ represents
   (a) C₄₋₅ cycloalkyl fused to a benzene ring and optionally substituted by one or more substituents selected from halo, C₁₋₄ alkyl, OH and C₁₋₄ alkoxy or
   (b) phenyl substituted as defined above in respect of aryl groups, except that the substituents include at least one OR^{10a} group in which R^{10a} is phenyl (which latter group is optionally substituted by one or more substituents selected from OH, halo, methyl and methoxy);
(5) R³ represents one to four substituents on the fused benzene ring, as defined above in respect of R³, except that the substituents include at least one OR^{7a} in which R^{7a} is other than H or C₁₋₁₀ alkyl (which latter group is optionally substituted as defined above in respect of R^{7a});
(6) R³ represents one to four (e.g. one or two) substituents on the fused benzene ring, as defined above in respect of R³, except that the substituents include at least one OR^{7a} in which R^{7a} is phenyl (which latter group is optionally substituted by one or more substituents selected from OH, halo, methyl and methoxy);
(7) R² represents unsubstituted C₁₋₃ alkyl, such as methyl.

For the avoidance of doubt, references herein to compounds of formula I include references to all embodiments described above in relation to compounds of formulae I, Ia and Ib.

The topical pharmaceutical composition according to the first aspect of the invention can be used to treat infections (e.g. infections comprising clinically latent microorganisms) and/or kill microorganisms (e.g. clinically latent microorganisms).

When used herein, the term “microorganisms” means:
(a) fungi (as defined below); and, particularly
(b) bacteria (as defined below).

References herein to the terms “microbial”, “antimicrobial” and “antimicrobially” shall be interpreted in accordance with the definition of “microorganisms”. For example, the term “microbial” means fungal or, particularly, bacterial.

When used herein, the terms “bacteria” (and derivatives thereof, such as “bacterial infection”) includes references to organisms (or infections due to organisms) of the following classes and specific types:

Gram-positive cocci, such as
Staphylococci (e.g. Staph. aureus, Staph. epidermidis, Staph. saprophyticus, Staph. auricularis, Staph. capitis capitis, Staph. c. ureolyticus, Staph. caprae, Staph. cohnii cohnii, Staph. c. urealyticus, Staph. equorum, Staph. gallinarum, Staph. haemolyticus, Staph. hominis hominis, Staph. h. novobiosepticius, Staph. hyicus, Staph. intermedius, Staph. lugdunensis, Staph. pasteuri, Staph. saccharolyticus, Staph. schleiferi schleiferi, Staph. s. coagulans, Staph. sciuri, Staph. simulans, Staph. warneri and Staph. xylosus) and
Streptococci (e.g. beta-haemolytic, pyogenic streptococci (such as Strept. agalactiae, Strept. canis, Strept. dysgalactiae dysgalactiae, Strept. dysgalactiae equisimilis, Strept. equi equi, Strept. equi zooepidemicus, Strept. iniae, Strept. porcinus and Strept. pyogenes),
   microaerophilic, pyogenic streptococci (Streptococcus “milleri”, such as Strept. anginosus, Strept. constellatus constellatus, Strept. constellatus pharyngidis and Strept. intermedius),
   oral streptococci of the “mitis” (alpha-haemolytic—Streptococcus “viridans”, such as Strept. mitis, Strept. oxalis, Strept. sanguinis, Strept. cristatus, Strept. gordonii and Strept. parasanguinis), “salivarius” (non-haemolytic, such as Strept. salivarius and Strept. vestibularis) and “mutans” (tooth-surface streptococci, such as Strept. criceti, Strept. mutans, Strept. ratti and Strept. sobrinus) groups,
   Strept. acidominimus, Strept. bovis, Strept. faecalis, Strept. equinus, Strept. pneumoniae and Strept. suis,
or Streptococci alternatively classified as Group A, B, C, D, E, G, L, P, U or V Streptococcus);

Gram-negative cocci, such as Neisseria gonorrhoeae, Neisseria meningitidis, Neisseria cinerea, Neisseria elongata, Neisseria flavescens, Neisseria lactamica, Neisseria mucosa, Neisseria sicca, Neisseria subflava and Neisseria weaveri;

Bacillaceae, such as Bacillus anthracis, Bacillus subtilis, Bacillus thuringiensis, Bacillus stearothermophilus and Bacillus cereus;

Enterobacteriaceae, such as
Escherichia coli,
Enterobacter (e.g. Enterobacter aerogenes, Enterobacter agglomerans and Enterobacter cloacae)
Citrobacter (such as Citrob. freundii and Citrob. divernis),
Hafnia (e.g. Hafnia alvei),
Erwinia (e.g. Erwinia persicinus),
Morganella morganii,
Salmonella (Salmonella enterica and Salmonella typhi),
Shigella (e.g. Shigella dysenteriae, Shigella flexneri, Shigella boydii and Shigella sonnei),
Klebsiella (e.g. Klebs. pneumoniae, Klebs. oxytoca, Klebs. ornitholytica, Klebs. planticola, Klebs. ozaenae, Klebs. terrigena, Klebs. granulomatis (Calymmatobacterium granulomatis) and Klebs. rhinoscleromatis),
Proteus (e.g. Pr. mirabilis, Pr. rettgeri and Pr. vulgaris),
Providencia (e.g. Providencia alcalifaciens, Providencia rettgeri and Providencia stuartii),
Serratia (e.g. Serratia marcescens and Serratia liquifaciens), and
Yersinia (e.g. Yersinia enterocolitica, Yersinia pestis and Yersinia pseudotuberculosis);

Enterococci (e.g. Enterococcus avium, Enterococcus casseliflavus, Enterococcus cecorum, Enterococcus dispar, Enterococcus durans, Enterococcus faecalis, Enterococcus faecium, Enterococcus flavescens, Enterococcus gallinarum, Enterococcus hirae, Enterococcus malodoratus, Enterococcus mundtii, Enterococcus pseudoavium, Enterococcus raffinosus and Enterococcus solitarius);

Helicobacter (e.g. Helicobacter pylori, Helicobacter cinaedi and Helicobacter fennelliae);

Acinetobacter (e.g. A. baumanii, A. calcoaceticus, A. haemolyticus, A. johnsonii, A. junii, A. lwoffi and A. radioresistens);

Pseudomonas (e.g. Ps. aeruginosa, Ps. maltophilia (Stenotrophomonas maltophilia), Ps. alcaligenes, Ps. chlororaphis, Ps. fluorescens, Ps. luteola. Ps. mendocina, Ps. monteilii, Ps. oryzihabitans, Ps. pertocinogena, Ps. pseudalcaligenes, Ps. putida and Ps. stutzeri);

Bacteriodes fragilis;

Peptococcus (e.g. Peptococcus niger);

Peptostreptococcus;

Clostridium (e.g. C. perfringens, C. difficile, C. botulinum, C. tetani, C. absonum, C. argentinense, C. baratii, C. bifermentans, C. beijerinckii, C. butyricum, C. cadaveric, C. carnis, C. celatum, C. clostridioforme, C. cochlearium, C. cocleatum, C. fallax, C. ghonii, C. glycolicum, C. haemolyticum, C. hastiforme, C. histolyticum, C. indolis, C. innocuum, C. irregulare, C. leptum, C. limosum, C. malenominatum, C. novyi, C. oroticum, C. paraputrificum, C. piliforme, C. putrefasciens, C. ramosum, C. septicum, C. sordelii, C. sphenoides, C. sporogenes, C. subterminale, C. symbiosum and C. tertium);

Mycoplasma (e.g. M. pneumoniae, M. hominis, M. genitalium and M. urealyticum);

Mycobacteria (e.g. Mycobacterium tuberculosis, Mycobacterium avium, Mycobacterium fortuitum, Mycobacterium marinum, Mycobacterium kansasii, Mycobacterium chelonae, Mycobacterium abscessus, Mycobacterium leprae, Mycobacterium smegmitis, Mycobacterium africanum, Mycobacterium alvei, Mycobacterium asiaticum, Mycobacterium aurum, Mycobacterium bohemicum, Mycobacterium bovis, Mycobacterium branderi, Mycobacterium brumae, Mycobacterium celatum, Mycobacterium chubense, Mycobacterium confluentis, Mycobacterium conspicuum, Mycobacterium cookii, Mycobacterium flavescens, Mycobacterium gadium, Mycobacterium gastri, Mycobacterium genavense, Mycobacterium gordonae, Mycobacterium goodii, Mycobacterium haemophilum, Mycobacterium hassicum, Mycobacterium intracellulare, Mycobacterium interjectum, Mycobacterium heidelberense, Mycobacterium lentiflavum, Mycobacterium malmoense, Mycobacterium microgenicum, Mycobacterium microti, Mycobacterium mucogenicum, Mycobacterium neoaurum, Mycobacterium nonchromogenicum, Mycobacterium peregrinum, Mycobacterium phlei, Mycobacterium scrofulaceum, Mycobacterium shimoidei, Mycobacterium simiae, Mycobacterium szulgai, Mycobacterium terrae, Mycobacterium thermoresistabile, Mycobacterium triplex, Mycobacterium triviale, Mycobacterium tusciae, Mycobacterium ulcerans, Mycobacterium vaccae, Mycobacterium wolinskyi and Mycobacterium xenopi);

Haemophilus (e.g. Haemophilus influenzae, Haemophilus ducreyi, Haemophilus aegyptius, Haemophilus parainfluenzae, Haemophilus haemolyticus and Haemophilus parahaemolyticus);

Actinobacillus (e.g. Actinobacillus actinomycetemcomitans, Actinobacillus equuli, Actinobacillus hominis, Actinobacillus lignieresii, Actinobacillus suis and Actinobacillus ureae);

Actinomyces (e.g. Actinomyces israelii);

Propionibacteria (e.g. Propionibacterium acnes);

Brucella (e.g. Brucella abortus, Brucella canis, Brucella melintensis and Brucella suis);

Campylobacter (e.g. Campylobacter jejuni, Campylobacter coli, Campylobacter lari and Campylobacter fetus);

Listeria monocytogenes;

Vibrio (e.g. Vibrio cholerae and Vibrio parahaemolyticus, Vibrio alginolyticus, Vibrio carchariae, Vibrio jiuvialis, Vibrio furnissii, Vibrio hollisae, Vibrio metschnikovii, Vibrio mimicus and Vibrio vulnificus);

Erysipelothrix rhusopathiae;

Corynebacteriaceae (e.g. Corynebacterium diphtheriae, Corynebacterium jeikeium and Corynebacterium urealyticum);

Spirochaetaceae, such as Borrelia (e.g. Borrelia recurrentis, Borrelia burgdorferi, Borrelia afzelii, Borrelia andersonii, Borrelia bissettii, Borrelia garinii, Borrelia japonica, Borrelia lusitaniae, Borrelia tanukii, Borrelia turdi, Borrelia valaisiana, Borrelia caucasica, Borrelia crocidurae, Borrelia duttoni, Borrelia graingeri, Borrelia hermsii, Borrelia hispanica, Borrelia latyschewii, Borrelia mazzottii, Borrelia parkeri, Borrelia persica, Borrelia turicatae and Borrelia venezuelensis) and Treponema (Treponema pallidum ssp. pallidum, Treponema pallidum ssp. endemicum, Treponema pallidum ssp. pertenue and Treponema carateum);

Pasteurella (e.g. Pasteurella aerogenes, Pasteurella bettyae, Pasteurella canis, Pasteurella dagmatis, Pasteurella gallinarum, Pasteurella haemolytica, Pasteurella multocida multocida, Pasteurella multocida gallicida, Pasteurella multocida septica, Pasteurella pneumotropica and Pasteurella stomatis);

Bordetella (e.g. Bordetella bronchiseptica, Bordetella hinzii, Bordetella holmseii, Bordetella parapertussis, Bordetella pertussis and Bordetella trematum);

Nocardiaceae, such as Nocardia (e.g. Nocardia asteroides and Nocardia brasiliensis);

Rickettsia (e.g. Ricksettsii or Coxiella burnetii);

Legionella (e.g. Legionalla anisa, Legionalla birminghamensis, Legionalla bozemanii, Legionalla cincinnatiensis, Legionalla dumoffii, Legionalla feeleii, Legionalla gormanii, Legionalla hackeliae, Legionalla israelensis, Legionalla jordanis, Legionalla lansingensis, Legionalla longbeachae, Legionalla maceachernii, Legionalla micdadei, Legionalla oakridgensis, Legionalla pneumophila, Legionalla sainthelensi, Legionalla tucsonensis and Legionalla wadsworthii);

Moraxella catarrhalis;

Stenotrophomonas maltophilia;

Burkholderia cepacia;

Francisella tularensis;

Gardnerella (e.g. Gardneralla vaginalis and Gardneralla mobiluncus);

Streptobacillus moniliformis;

Flavobacteriaceae, such as Capnocytophaga (e.g. Capnocytophaga canimorsus, Capnocytophaga cynodegmi, Capnocytophaga gingivalis, Capnocytophaga granulosa, Capnocytophaga haemolytica, Capnocytophaga ochracea and Capnocytophaga sputigena);

Bartonella (Bartonella bacilliformis, Bartonella clarridgeiae, Bartonella elizabethae, Bartonella henselae, Bartonella quintana and Bartonella vinsonii arupensis);

Leptospira (e.g. Leptospira biflexa, Leptospira borgpetersenii, Leptospira inadai, Leptospira interrogans, Leptospira kirschneri, Leptospira noguchii, Leptospira santarosai and Leptospira weilii);

Spirillium (e.g. Spirillum minus);

Bacteroides (e.g. Bacteroides caccae, Bacteroides capillosus, Bacteroides coagulans, Bacteroides distasonis, Bacteroides eggerthii, Bacteroides forsythus, Bacteroides fragilis, Bacteroides merdae, Bacteroides ovatus, Bacteroides putredinis, Bacteroides pyogenes, Bacteroides splanchinicus, Bacteroides stercoris, Bacteroides tectus, Bacteroides thetaiotaomicron, Bacteroides uniformis, Bacteroides ureolyticus and Bacteroides vulgatus);

Prevotella (e.g. Prevotella bivia, Prevotella buccae, Prevotella corporis, Prevotella dentalis (Mitsuokella dentalis), Prevotella denticola, Prevotella disiens, Prevotella enoeca, Prevotella heparinolytica, Prevotella intermedia, Prevotella loeschii, Prevotella melaninogenica, Prevotella nigrescens, Prevotella oralis, Prevotella oris, Prevotella oulora, Prevotella tannerae, Prevotella venoralis and Prevotella zoogleoformans);

Porphyromonas (e.g. Porphyromonas asaccharolytica, Porphyromonas cangingivalis, Porphyromonas canoris, Porphyromonas cansulci, Porphyromonas catoniae, Porphyromonas circumdentaria, Porphyromonas crevioricanis, Porphyromonas endodontalis, Porphyromonas gingivalis, Porphyromonas gingivicanis, Porphyromonas levii and Porphyromonas macacae);

Fusobacterium (e.g. F. gonadiaformans, F. mortiferum, F. naviforme, F. necrogenes, F. necrophorum necrophorum, F. necrophorum fundiliforme, F. nucleatum nucleatum, F. nucleatum fusiforme, F. nucleatum polymorphum, F. nucleatum vincentii, F. periodonticum, F. russii, F. ulcerans and F. varium);

Chlamydia (e.g. Chlamydia trachomatis);

Chlamydophila (e.g. Chlamydophila abortus (Chlamydia psittaci), Chlamydophila pneumoniae (Chlamydia pneumoniae) and Chlamydophila psittaci (Chlamydia psittaci));

Leuconostoc (e.g. Leuconostoc citreum, Leuconostoc cremoris, Leuconostoc dextranicum, Leuconostoc lactis, Leuconostoc mesenteroides and Leuconostoc pseudomesenteroides);

Gemella (e.g. Gemella bergeri, Gemella haemolysans, Gemella morbillorum and Gemella sanguinis); and

Ureaplasma (e.g. Ureaplasma parvum and Ureaplasma urealyticum).

When used herein, the terms “fungi” (and derivatives thereof, such as “fungal infection”) includes references to organisms (or infections due to organisms) of the following classes and specific types:

Absidia (e.g. Absidia corymbifera);

Ajellomyces (e.g. Ajellomyces capsulatus and Ajellomyces dermatitidis);

Arthroderma (e.g. Arthroderma benhamiae, Arthroderma fulvum, Arthroderma gypseum, Arthroderma incurvatum, Arthroderma otae and Arthroderma vanbreuseghemii);

Aspergillus (e.g. Aspergillus flavus, Aspergillus fumigatus, Aspergillus niger and Aspergillus terreus, such as any species other than the latter);

Blastomyces (e.g. Blastomyces dermatitidis);

Candida (e.g. Candida albicans, Candida glabrata, Candida guilliermondii, Candida krusei, Candida parapsilosis, Candida tropicalis, Candida pelliculosa and Candida lusitaniae, such as any species other than the latter);

Cladophialophora (e.g. Cladophialophora carrionii);

Coccidioides (e.g. Coccidioides immitis);

Cryptococcus (e.g. Cryptococcus neoformans);

Cunninghamella (e.g. Cunninghamella sp.)

Epidermophyton (e.g. Epidermophyton floccosum);

Exophiala (e.g. Exophiala dermatitidis);

Filobasidiella (e.g. Filobasidiella neoformans);

Fonsecaea (e.g. Fonsecaea pedrosoi);

Fusarium (e.g. Fusarium solani and Fusarium oxysporum, such as the former species);

Geotrichum (e.g. Geotrichum candidum);

Histoplasma (e.g. Histoplasma capsulatum);

Hortaea (e.g. Hortaea werneckii);

Issatchenkia (e.g. Issatchenkia orientalis);

Madurella (e.g. Madurella grisae);

Malassezia (otherwise known as Pityrosporum) (e.g. Malassezia furfur, Malassezia globosa, Malassezia obtusa, Malassezia pachydermatis, Malassezia restricta, Malassezia slooffiae, Malassezia sympodialis, Malassezia dermatis, Malassezia nana and Malassezia yamatoensis, such as any species other than the latter three);

Microsporum (e.g. Microsporum canis, Microsporum fulvum, Microsporum gypseum, Microsporum audouinii and Microsporum ferrugineum, such as any one of the three former species);

Mucor (e.g. Mucor circinelloides);

Nectria (e.g. Nectria haematococca);

Paecilomyces (e.g. Paecilomyces variotii);

Paracoccidioides (e.g. Paracoccidioides brasiliensis);

Penicillium (e.g. Penicillium marneffei);

Pichia (e.g. Pichia anomala and Pichia guilliermondii);

Pneumocystis (e.g. Pneumocystis jiroveci (Pneumocystis carinii));

Pseudallescheria (e.g. Pseudallescheria boydii);

Rhizopus (e.g. Rhizopus oryzae and Rhizopus oligosporus, such as the former species);

Rhodotorula (e.g. Rhodotorula rubra);

Scedosporium (e.g. Scedosporium apiospermum);

Schizophyllum (e.g. Schizophyllum commune);

Sporothrix (e.g. Sporothrix schenckii);

Trichophyton (e.g. Trichophyton mentagrophytes, Trichophyton rubrum, Trichophyton verrucosum, Trichophyton violaceum, Trichophyton schoenleinii, Trichophyton tonsurans, Trichophyton concentricum, Trichophyton gourvilii, Trichophyton interdigitale, Trichophyton megninii, Trichophyton soudanense and Trichophyton yaoundei, such as any one of the four former species); and

Trichosporon (e.g. Trichosporon asahii, Trichosporon cutaneum, Trichosporon inkin and Trichosporon mucoides).

Particular bacteria that may be mentioned include:
(i) Staphylococci, such as Staph. aureus (either Methicillin-sensitive (i.e. MSSA) or Methicillin-resistant (i.e. MRSA)), Staph. epidermidis, Staphylococcus haemolyticus, Staphylococcus hominis and Staphylococcus lugdunensis (e.g. Staph. aureus and Staph. epidermidis);
(ii) Streptococci, such as Strept. agalactiae, Strept. pyogenes; Strept. pneumoniae, and Strept. Group C (e.g. either of the two former species);

(iii) Bacillaceae, such as Bacillus anthracis or Bacillus cereus (e.g. the former species);
(iv) Enterobacteriaceae, such as Escherichia coli, Klebsiella (e.g. Klebs. pneumoniae and Klebs. oxytoca) and Proteus (e.g. Pr. mirabilis, Pr. rettgeri and Pr. vulgaris);
(v) Haemophilus influenzae;
(vi) Enterococci, such as Enterococcus faecalis, Enterococcus faecium, Enterococcus gallinarum and Enterococcus casseliflavus (e.g. either of the two former species);
(vii) Mycobacteria, such as Mycobacterium tuberculosis;
(viii) Propionibacteria, such as Propionibacterium acnes;
(ix) Corynebacteriaceae such as Corynebacterium jeikeium;
(x) Stenotrophomonas maltophilia; and
(xi) Mycoplasma such as M. pneumoniae.

Certain bacteria that may be mentioned include those at (i) to (vii) above. However, other bacteria that may be mentioned in particular include those at (i), (ii) and (viii) above.

Particular fungi that may also be mentioned in this respect include:
(I) Aspergillus (e.g. Aspergillus fumigatus; Aspergillus niger, Aspergillus flavus or Aspergillus terreus, such as the former species);
(II) Candida (e.g. Candida albicans, Candida tropicalis, Candida parapsilosis, Candida glabrata or Candida lusitaniae, such as the former species);
(III) Cryptococcus neoformans;
(IV) Histoplasma capsulatum;
(V) Pneumocystis jiroveci;
(VI) Issatchenkia orientalis;
(VII) Rhizopus oligosporus;
(VIII) Fusarium oxysporum;
(IX) Microsporum (e.g. Microsporum audouinii, Microsporum ferrugineum or, particularly, Microsporum canis);
(X) Epidermophyton floccosum;
(XI) Malassezia (e.g. Malassezia furfur); and
(XII) Trichophyton (e.g. Trichophyton mentagrophytes, Trichophyton rubrum, Trichophyton verrucosum, Trichophyton violaceum, Trichophyton schoenleinii, Trichophyton tonsurans, Trichophyton concentricum, Trichophyton gourvilii, Trichophyton interdigitale, Trichophyton megninii, Trichophyton soudanense and Trichophyton yaoundei (such as Trichophyton violaceum, Trichophyton mentagrophytes or, particularly, Trichophyton rubrum)).

Certain fungi that may be mentioned include those at (I) to (V) above. However, other fungi that may be mentioned in particular include those at (I), (II), (X), (XI) and (XII) above.

Particular conditions involving microorganisms that may be mentioned include tuberculosis (e.g. pulmonary tuberculosis, non-pulmonary tuberculosis (such as genito-urinary tuberculosis) and miliary tuberculosis), anthrax, abscesses, acne vulgaris, actinomycosis, bacterial conjunctivitis, bacterial keratitis, Buruli ulcer, bronchitis (acute or chronic), burn wounds, cat scratch fever, cellulitis, chancroid, cutaneous diphtheria, cystic fibrosis, cystitis, diffuse panbronchiolitis, diphtheria, dental caries, diseases of the upper respiratory tract, epiglottitis, erysipelas, erysipeloid, erythrasma, eye infections, furuncles, Gardnerella vaginitis, gastrointestinal infections (gastroenteritis), genital infections, gingivitis, gonorrhoea, granuloma inguinale, Haverhill fever, infected burns, infections following dental operations, infections in the oral region, infections associated with prostheses, leprosy, lymphogranuloma venerium, mastitis, mycetoma, nocardiosis (e.g. Madura foot), non-specific urethritis, opthalmia (e.g. opthalmia neonatorum), otitis (e.g. otitis externa and otitis media), paronychia, pharyngitis, phlegmons, pinta, plague, pneumonia, postoperative wound infections, postoperative gas gangrene, prostatitis, pulmonary emphysema, pyoderma (e.g. impetigo), Q fever, rat-bite fever, Ritter's disease, septic infections, sinusitis, skin infections (e.g. skin granulomas), syphilis, tonsillitis, trachoma, urethritis, wound infections, yaws, aspergillosis, candidiasis (e.g. oropharyngeal candidiasis, vaginal candidiasis or balanitis), cryptococcosis, favus, histoplasmosis, intertrigo, mucormycosis, tinea (e.g. tinea corporis, tinea capitis, tinea cruris, tinea pedis and tinea unguium), onychomycosis, pityriasis versicolor, ringworm and sporotrichosis.

Further conditions that may be mentioned include infections with MSSA, MRSA, Staph. epidermidis, Strept. agalactiae, Strept. pyogenes, Escherichia coli, Klebs. pneumoniae, Klebs. oxytoca, Pr. mirabilis, Pr. rettgeri, Pr. vulgaris, Haemophilus influenzae, Enterococcus faecalis or Enterococcus faecium.

When used herein, the term “clinically latent” includes references to microorganisms that are viable but non-culturable (e.g. bacteria that cannot be detected by standard culture techniques but that are detectable and quantifiable by techniques such as broth dilution counting, microscopy, or molecular techniques such as polymerase chain reaction).

The term “clinically latent” also includes references to microorganisms that are phenotypically tolerant, for example microorganisms that:
(a) are sensitive (e.g. in log phase) to the biostatic (e.g. bacteriostatic) effects of conventional antimicrobial agents (i.e. microorganisms for which the minimum inhibitory concentration (MIC) of a conventional antimicrobial is substantially unchanged); but
(b) possess drastically decreased susceptibility to drug-induced killing (e.g. microorganisms for which, with any given conventional antimicrobial agent, the ratio of minimum microbicidal concentration (e.g. minimum bactericidal concentration, MBC) to MIC is 10 or more).

In relation to point (a) above, “substantially unchanged” refers to MIC values that are anywhere from 50 to 200% (e.g. 90 to 110%) of the value determined under standard conditions for the microorganism and conventional antimicrobial agent concerned.

For the avoidance of doubt, the term “clinically latent” excludes references to microorganisms that are genotypically resistant to conventional antimicrobial agents (i.e. microorganisms that differ genetically from antimicrobial-sensitive members of the same genus and that display an increased MIC (e.g. in log phase) for one or more conventional antimicrobial agents compared to said antimicrobial-sensitive microorganisms).

The term “clinically latent” further includes references to microorganisms that:
are metabolically active; but
(ii) have a growth rate that is below the threshold of infectious disease expression.

The term “threshold of infectious disease expression” will be understood by those skilled in the art to include references to the growth rate threshold below which the symptoms of infectious disease (in a patient infected with the relevant microorganism) are absent.

In relation to point (i) above, metabolic activity of latent microorganisms can be determined by several methods known to those skilled in the art, for example by measuring mRNA levels in the microorganisms or by determining their rate of uridine uptake. In this respect, the term “clinically latent” further includes references to microorganisms that, compared to the same number of microorganisms under logarithmic growth conditions (in vitro or in vivo), possess reduced but still significant levels of:
(I) mRNA (e.g. from 0.0001 to 50%, such as from 1 to 30, 5 to 25 or 10 to 20%, of the level of mRNA); and/or
(II) uridine (e.g. [³H]uridine) uptake (e.g. from 0.0005 to 50%, such as from 1 to 40, 15 to 35 or 20 to 30% of the level of [³H]uridine uptake).

When used herein, the term “conventional antimicrobial agent(s)” means:
(a) conventional antifungal agents; and, particularly
(b) conventional antibacterial agents,
   wherein each of (a) and (b) is as defined below.

When used herein, the term “conventional antibacterial agent(s)” include references to bactericidal and bacteristatic agents that are known in the prior art (i.e. agents that have been selected and developed on the basis of their MICs—namely their ability to inhibit the growth of bacteria). In this respect, particular conventional antibacterial agents that may be mentioned include any one or more of the following.
(a) β-Lactams, including:
   (i) penicillins, such as
      (I) benzylpenicillin, procaine benzylpenicillin, phenoxy-methylpenicillin, methicillin, propicillin, epicillin, cyclacillin, hetacillin, 6-aminopenicillanic acid, penicillic acid, penicillanic acid sulphone (sulbactam), penicillin G, penicillin V, phenethicillin, phenoxymethylpenicillinic acid, azlocillin, carbenicillin, cloxacillin, D-(−)-penicillamine, dicloxacillin, nafcillin and oxacillin,
      (II) penicillinase-resistant penicillins (e.g. flucloxacillin),
      (III) broad-spectrum penicillins (e.g. ampicillin, amoxicillin, metampicillin and bacampicillin),
      (IV) antipseudomonal penicillins (e.g. carboxypenicillins such as ticarcillin or ureidopenicillins such as piperacillin),
      (V) mecillinams (e.g. pivmecillinam), or
      (VI) combinations of any two or more of the agents mentioned at (I) to (V) above, or combinations of any of the agents mentioned at (I) to (V) above with a β-lactamase inhibitor such as tazobactam or, particularly, clavulanic acid (which acid is optionally in metal salt form, e.g. in salt form with an alkali metal such as sodium or, particularly, potassium);
   (ii) cephalosporins, such as cefaclor, cefadroxil, cefalexin (cephalexin), cefcapene, cefcapene pivoxil, cefdinir, cefditoren, cefditoren pivoxil, cefixime, cefotaxime, cefpirome, cefpodoxime, cefpodoxime proxetil, cefprozil, cefradine, ceftazidime, cefteram, cefteram pivoxil, ceftriaxone, cefuroxime, cefuroxime axetil, cephaloridine, cephacetrile, cephamandole, cephaloglycine, ceftobiprole, PPI-0903 (TAK-599), 7-aminocephalosporanic acid, 7-aminodes-acetoxycephalosporanic acid, cefamandole, cefazolin, cefmetazole, cefoperazone, cefsulodin, cephalosporin C zinc salt, cephalothin, cephapirin; and
   (iii) other β-lactams, such as monobactams (e.g. aztreonam), carbapenems (e.g. imipenem (optionally in combination with a renal enzyme inhibitor such as cilastatin), meropenem, ertapenem, doripenem (S-4661) and R⁰⁴⁹⁰⁸⁴⁶³ (CS-023)), penems (e.g. faropenem) and 1-oxa-β-lactams (e.g. moxalactam).
(b) Tetracyclines, such as tetracycline, demeclocycline, doxycycline, lymecycline, minocycline, oxytetracycline, chlortetracycline, meclocycline and methacycline, as well as glycylcyclines (e.g. tigecycline).
(c) Aminoglycosides, such as amikacin, gentamicin, netilmicin, neomycin, streptomycin, tobramycin, amastatin, butirosin, butirosin A, daunorubicin, dibekacin, dihydrostreptomycin, G 418, hygromycin B, kanamycin B, kanamycin, kirromycin, paromomycin, ribostamycin, sisomicin, spectinomycin, streptozocin and thiostrepton.
(d) (i) Macrolides, such as azithromycin, clarithromycin, erythromycin, roxithromycin, spiramycin, amphotericins B (e.g. amphotericin B), bafilomycins (e.g. bafilomycin A1), brefeldins (e.g. brefeldin A), concanamycins (e.g. concanamycin A), filipin complex, josamycin, mepartricin, midecamycin, nonactin, nystatin, oleandomycin, oligomycins (e.g. oligomycin A, oligomycin B and oligomycin C), pimaricin, rifampicin, rifamycin, rosamicin, tylosin, virginiamycin and fosfomycin.
(ii) Ketolides such as telithromycin and cethromycin (ABT-773).
(iii) Lincosamines, such as lincomycin.
(e) Clindamycin and clindamycin 2-phosphate.
Phenicols, such as chloramphenicol and thiamphenicol.
(g) Steroids, such as fusidic acid (optionally in metal salt form, e.g. in salt form with an alkali metal such as sodium).
(h) Glycopeptides such as vancomycin, teicoplanin, bleomycin, phleomycin, ristomycin, telavancin, dalbavancin and oritavancin.
(i) Oxazolidinones, such as linezolid and AZD2563.
Streptogramins, such as quinupristin and dalfopristin, or a combination thereof
(k) (i) Peptides, such as polymyxins (e.g. colistin and polymyxin B), lysostaphin, duramycin, actinomycins (e.g. actinomycin C and actinomycin D), actinonin, 7-aminoactinomycin D, antimycin A, antipain, bacitracin, cyclosporin A, echinomycin, gramicidins (e.g. gramicidin A and gramicidin C), myxothiazol, nisin, paracelsin, valinomycin and viomycin.
(ii) Lipopeptides, such as daptomycin.
(iii) Lipoglycopeptides, such as ramoplanin.
(l) Sulfonamides, such as sulfamethoxazole, sulfadiazine, sulfaquinoxaline, sulfathiazole (which latter two agents are optionally in metal salt form, e.g. in salt form with an alkali metal such as sodium), succinylsulfathiazole, sulfadimethoxine, sulfaguanidine, sulfamethazine, sulfamonomethoxine, sulfanilamide and sulfasalazine.
(m) Trimethoprim, optionally in combination with a sulfonamide, such as sulfamethoxazole (e.g. the combination co-trimoxazole).
(n) Antituberculous drugs, such as isoniazid, rifampicin, rifabutin, pyrazinamide, ethambutol, streptomycin, amikacin, capreomycin, kanamycin, quinolones (e.g. those at (q) below), para-aminosalicylic acid, cycloserine and ethionamide.
(o) Antileprotic drugs, such as dapsone, rifampicin and clofazimine.
(p) (i) Nitroimidazoles, such as metronidazole and tinidazole.
(ii) Nitrofurans, such as nitrofurantoin.
(q) Quinolones, such as nalidixic acid, norfloxacin, ciprofloxacin, ofloxacin, levofloxacin, moxifloxacin, gatifloxacin, gemifloxacin, garenoxacin, DX-619, WCK 771 (the arginine salt of S-(−)-nadifloxacin), 8-quinolinol, cinoxacin, enrofloxacin, flumequine, lomefloxacin, oxolinic acid and pipemidic acid.
(r) Amino acid derivatives, such as azaserine, bestatin, D-cycloserine, 1,10-phenanthroline, 6-diazo-5-oxo-L-norleucine and L-alanyl-L-1-aminoethyl-phosphonic acid.
(s) Aureolic acids, such as chromomycin A3, mithramycin A and mitomycin

C.
(t) Benzochinoides, such as herbimycin A.
(u) Coumarin-glycosides, such as novobiocin.
(v) Diphenyl ether derivatives, such as irgasan.
(w) Epipolythiodixopiperazines, such as gliotoxin from Gliocladium fimbriatum.
(x) Fatty acid derivatives, such as cerulenin.
(y) Glucosamines, such as 1-deoxymannojirimycin, 1-deoxynojirimycin and N-methyl-1-deoxynojirimycin.
(z) Indole derivatives, such as staurosporine.
(aa) Diaminopyrimidines, such as iclaprim (AR-100).
(ab) Macrolactams, such as ascomycin.
(ac) Taxoids, such as paclitaxel.
(ad) Statins, such as mevastatin.
(ae) Polyphenolic acids, such as (+)-usnic acid.
(af) Polyethers, such as lasalocid A, lonomycin A, monensin, nigericin and salinomycin.
(ag) Picolinic acid derivatives, such as fusaric acid.
(ah) Peptidyl nucleosides, such as blasticidine S, nikkomycin, nourseothricin and puromycin.
(ai) Nucleosides, such as adenine 9-β-D-arabinofuranoside, 5-azacytidine, cordycepin, formycin A, tubercidin and tunicamycin.
(aj) Pleuromutilins, such as GSK-565154, GSK-275833 and tiamulin.
(ak) Peptide deformylase inhibitors, such as LBM415 (NVP PDF-713) and BB 83698.
(al) Antibacterial agents for the skin, such as fucidin, benzamycin, clindamycin, erythromycin, tetracycline, silver sulfadiazine, chlortetracycline, metronidazole, mupirocin, framycitin, gramicidin, neomycin sulfate, polymyxins (e.g. polymixin B) and gentamycin;
(am) Miscellaneous agents, such as methenamine (hexamine), doxorubicin, piericidin A, stigmatellin, actidione, anisomycin, apramycin, coumeimycin A1, L(+)-lactic acid, cytochalasins (e.g. cytochalasin B and cytochalasin D), emetine and ionomycin.

Particular conventional antibacterial agents that may be mentioned include those listed at (al) above.

When used herein, the term “conventional antifungal agent(s)” include references to fungicidal and fungistatic agents that are known in the prior art (i.e. agents that have been selected and developed on the basis of their MICs—namely their ability to inhibit the growth of fungi). In this respect, particular conventional antifungal agents that may be mentioned include any one or more of the following.
(a) azole antifungals, such as imidazoles (e.g. clotrimazole, econazole, fenticonazole, ketoconazole, miconazole, sulconazole, and tioconazole) or triazoles (e.g. fluconazole, itraconazole and voriconazole);
(b) polyene antifungals, such as amphotericin and nystatin;
(c) miscellaneous antifungal agents such as griseofulvin, caspofungin or flucytosine, which latter two agents are optionally employed in combination;
(d) allylamine antifungals, such as terbinafine.

In treating treat infections and killing microorganisms, the compound of formula I can be employed as the sole antimicrobial agent in the topical pharmaceutical composition. Alternatively, the compound of formula I can be used in combination with a conventional antimicrobial agent.

Thus, according to a second aspect of the invention, there is provided a combination product for topical administration comprising:
(A) a compound of formula I, as hereinbefore defined, or a pharmaceutically-acceptable derivative thereof; and
(B) a conventional antimicrobial agent, as hereinbefore defined, or a pharmaceutically-acceptable derivative thereof, and/or
   a conventional sterilising agent, as defined below,
      wherein each of components (A) and (B) is formulated in admixture with a pharmaceutically-acceptable adjuvant, diluent or carrier.

The term “conventional sterilising agent”, when used herein, includes references to alcohols (e.g. industrial methylated spirits or ethanol), sodium chloride, thymol, chlorhexidine, cationic surfactants (e.g. cetrimide), iodine (optionally combined with povidone), phenolics (e.g. triclosan), oxidants (e.g. hydrogen peroxide, potassium permanganate or sodium hypochlorite) and any one or more of the conventional antimicrobial agents described above.

The combination product provides for the administration of component (A) in conjunction with component (B), and may thus be presented either as separate topical formulations, wherein at least one of those formulations comprises component (A) and at least one comprises component (B), or may be presented (i.e. formulated) as a combined topical preparation (i.e. presented as a single topical formulation including component (A) and component (B)).

Thus, there is further provided:
(1) a topical pharmaceutical composition including a compound of formula I, as hereinbefore defined, or a pharmaceutically-acceptable derivative thereof, and
   a conventional antimicrobial agent, as hereinbefore defined, or a pharmaceutically-acceptable derivative thereof and/or
   a conventional sterilising agent, as hereinbefore defined, in admixture with a pharmaceutically-acceptable adjuvant, diluent or carrier (which formulation is hereinafter referred to as a “combined preparation”); and
(2) a kit of parts comprising components:
   (I) a topical pharmaceutical composition including a compound of formula I, as hereinbefore defined, or a pharmaceutically-acceptable derivative thereof, in admixture with a pharmaceutically-acceptable adjuvant, diluent or carrier; and
   (II) a topical pharmaceutical formulation including
   a conventional antimicrobial agent, as hereinbefore defined, or a pharmaceutically-acceptable derivative thereof and/or
   a conventional sterilising agent, as hereinbefore defined in admixture with a pharmaceutically-acceptable adjuvant, diluent or carrier,
   which components (I) and (II) are each provided in a form that is suitable for administration in conjunction with the other.

Component (I) of the kit of parts is thus component (A) in admixture with a pharmaceutically-acceptable adjuvant, diluent or carrier. Similarly, component (II) is component (B) in admixture with a phal naceutically-acceptable adjuvant, diluent or carrier.

The invention also encompasses a method of making a kit of parts as defined above, which method comprises bringing a component (I), as defined above, into association with a component (II), as defined above, thus rendering the two components suitable for topical administration in conjunction with each other.

By bringing the two components “into association with” each other, we include that components (I) and (II) of the kit of parts may be:
(i) provided as separate formulations (i.e. independently of one another), which are subsequently brought together for use in conjunction with each other in combination therapy; or
(ii) packaged and presented together as separate components of a “combination pack” for use in conjunction with each other in combination therapy.

Thus, there is further provided a kit of parts comprising:
(1) one of components (I) and (II) as defined herein; together with
(2) instructions to use that component in conjunction with the other of the two components.

The kits of parts described herein may comprise more than one formulation including an appropriate quantity/dose of component (A), and/or more than one formulation including an appropriate quantity/dose of component (B), in order to provide for repeat dosing. If more than one formulation (comprising either active compound) is present, such formulations may be the same, or may be different in terms of the dose of component (A) or component (B), chemical composition and/or physical form.

When used herein, the term “topical” includes references to formulations that are adapted for application to body surfaces (e.g. the skin or mucous membranes).

Mucous membranes that may be mentioned in this respect include the mucosa of the vagina, the penis, the urethra, the bladder, the anus, the mouth (including the mucosa of the cheek, the soft palate, the under surface of tongue and the floor of the mouth), the nose, the throat (including the mucosa of the pharynx, the larynx, the trachea and the esophagus), the bronchi, the lungs, the eye and the ear.

Thus, in certain embodiments of the first and second aspects of the invention, the topical pharmaceutical composition or combination product is, for example, an intravaginal, an intraurethral, an intravesical, a buccal or, particularly, an intranasal composition or product (i.e. is specifically adapted for intravaginal, intraurethral, intravesical, buccal or, particularly, intranasal administration).

Thus, the present invention also encompasses intranasal, buccal, intraurethral, intravesical and intravaginal compositions comprising a compound of formula I, as hereinbefore defined, or a pharmaceutically-acceptable derivative thereof, in admixture with a pharmaceutically acceptable adjuvant, diluent or carrier.

Similarly, the present invention also encompasses combination products for intranasal, buccal, intraurethral, intravesical or intravaginal administration comprising:
(A) a compound of formula I, as hereinbefore defined, or a pharmaceutically-acceptable derivative thereof; and
(B) a conventional antimicrobial agent, as hereinbefore defined,
   wherein each of components (A) and (B) is formulated in admixture with a pharmaceutically-acceptable adjuvant, diluent or carrier.

As for the combination product according to the secod aspect of the invention, this combination product provides for the administration of component (A) in conjunction with component (B), and may thus be presented either as separate topical formulations, wherein at least one of those formulations comprises component (A) and at least one comprises component (B), or may be presented (i.e. formulated) as a combined topical preparation (i.e. presented as a single topical formulation including component (A) and component (B)).

In alternative embodiments, the invention also relates to a mouthwash, or a formulation for inhalation, comprising a compound of formula I, as hereinbefore defined, or a pharmaceutically-acceptable derivative thereof, in admixture with a pharmaceutically acceptable adjuvant, diluent or carrier.

Further, alternative embodiments of the present invention also encompass a mouthwash, or a formulation for inhalation, comprising a compound of formula I, as hereinbefore defined, or a pharmaceutically-acceptable derivative thereof and a conventional antimicrobial agent, as hereinbefore defined.

Topical compositions, which are useful for treating disorders of the skin or of membranes (e.g. those accessible by digitation, such as membranes of the mouth, vagina, cervix, anus and rectum), include creams, ointments, lotions, sprays, gels and sterile aqueous solutions or suspensions. As such, topical compositions include those in which the active ingredient(s) is (are) dissolved or dispersed in a dermatological vehicle known in the art (e.g. aqueous or non-aqueous gels, ointments, water-in-oil or oil-in-water emulsions). Constituents of such vehicles may comprise water, aqueous buffer solutions, non-aqueous solvents (such as ethanol, isopropanol, benzyl alcohol, 2-(2-ethoxyethoxy)ethanol, propylene glycol, propylene glycol monolaurate, glycofurol or glycerol), oils (e.g. a mineral oil such as a liquid paraffin, natural or synthetic triglycerides such as Miglyol™, or silicone oils such as dimethicone). Depending, inter alia, upon the nature of the formulation as well as its intended use and site of application, the dermatological vehicle employed may contain one or more components (for example, when the formulation is an aqueous gel, components in addition to water) selected from the following list:

a solubilising agent or solvent (e.g. a (3-cyclodextrin, such as hydroxypropyl P-cyclodextrin, or an alcohol or polyol such as ethanol, propylene glycol or glycerol);

a thickening agent (e.g. hydroxyethylcellulose, hydroxypropylcellulose, carboxymethylcellulose or carbomer);

a gelling agent (e.g. a polyoxyethylene-polyoxypropylene copolymer);

a preservative (e.g. benzyl alcohol, benzalkonium chloride, chlorhexidine, chlorbutol, a benzoate, potassium sorbate or EDTA or salt thereof); and

pH buffering agent(s) (such as a mixture of dihydrogen phosphate and hydrogen phosphate salts, or a mixture of citric acid and a hydrogen phosphate salt).

The amount of compound of formula I, Ia or Ib used in topical compositions or combination products will depend, inter alia, upon the particular nature of the composition or combination product, as well as its intended use. In any event, those skilled in the art will be able to determine, by routine and non-inventive methods, amounts of compound of formula I, Ia or Ib that can be employed. Typically, however, the compound of formula I, Ia or Ib will be present in the topical composition or combination product at from 0.01 to 25% by weight (e.g. from 0.1 to 10% by weight, such as from 0.1 to 5% by weight or, particularly, from 0.5 to 3% (e.g. 2% or, particularly, 1%) by weight) of the composition or product.

In certain embodiments of the first aspect of the invention, the topical compositions comprises a compound of formula I (e.g. at 0.5 to 3%, such as 2% or 1%, by weight) and:
(a) water;
(b) one or more polar, non-aqueous solvents (e.g. an alcohol or polyol such as ethanol, propylene glycol and/or glycerol);
(c) a preservative (e.g. benzyl alcohol);
(d) a thickening agent (e.g. hydroxyethylcellulose); and, optionally
(e) pH buffering agent(s) (such as a mixture of dihydrogen phosphate and hydrogen phosphate salts).

In particular compositions, and depending, inter alia, upon the amount of compound of formula I present (typically, the higher the amount of the compound of formula I, the larger the amount of polar, non-aqueous solvents required to solublise the compound):
(i) water may be present at from 55 to 75% (e.g. from 60 to 72.5%) by weight;
(ii) the one or more polar, non-aqueous solvents may (together) be present at from 15 to 40% (e.g. from 24 to 35%) by weight;
(iii) glycerol, if used, may be present at from 5 to 25% (e.g. from 15 to 20%) by weight;
(iv) ethanol, if used, may be present at from 3 to 10% (e.g. from 5 to 8%) by weight;
(v) propylene glycol, if used, may be present at from 2 to 15% (e.g. from 4 to 6%) by weight;
(vi) the preservative may be present at from 0.1 to 3% (e.g. about 1%) by weight;
(vii) the thickening agent may be present at from 1 to 5% (e.g. about 2% by weight).

In further particular topical compositions, the pH buffering agent(s) may, if employed and when dissolved in the water component of the composition, provide a pH in the range of 5 to 7 (e.g. about pH 5.5).

Methods of producing topical pharmaceutical compositions (including intranasal, buccal, intraurethral, intravesical and intravaginal compositions, as well as mouthwashes and formulations for inhalation) such as creams, ointments, lotions, sprays and sterile aqueous solutions or suspensions are well known in the art. Suitable methods of preparing topical pharmaceutical compositions are described, for example in WO 95/10999, U.S. Pat. No. 6,974,585, WO 2006/048747, as well as in documents cited in any of these references.

Typically, the topical pharmaceutical compositions and combination products according to the present invention can be prepared by mixing together the components of the compositions or (parts of) products. However, for topical compositions comprising a mixture of aqueous and non-aqueous components, the composition may, in particular embodiments, be prepared by:
(a) dissolving the compound of formula I, Ia or Ib in one or more non-aqueous solvents (e.g. one or more polar, non-aqueous solvents, such as one or more solvents selected from alcohols (e.g. ethanol) and polyols (e.g. propylene glycol and/or glycerol), optionally in combination with a preservative (e.g. benzyl alcohol));
(b) adding the aqueous component (e.g. either water or a buffered aqueous solution); and, if a gel is required,
(c) adding a gelling agent (e.g. a polyoxyethylene-polyoxypropylene copolymer) or, particularly, a thickening agent (e.g. hydroxyethyl-cellulose).

Topical pharmaceutical compositions and combination products according to the present invention may be used to treat a variety of skin or membrane disorders, such as infections of the skin or membranes (e.g. e.g. infections of nasal membranes, axilla, groin, perineum, rectum, dermatitic skin, skin ulcers, and sites of insertion of medical equipment such as i.v. needles, catheters and tracheostomy or feeding tubes) with any of the bacteria or fungi described hereinbefore, (e.g. any of the Staphylococci, Streptococci, Mycobacteria or Pseudomonas organisms mentioned hereinbefore, such as S. aureus (e.g. Methicillin resistant S. aureus (MRSA))).

Particular bacterial conditions that may be treated by topical pharmaceutical compositions and combination products according to the present invention also include the skin- and membrane-related conditions disclosed hereinbefore, as well as: acne vulgaris; acne rosacea; rosacea (including erythematotelangiectatic rosacea, papulopustular rosacea, phymatous rosacea and ocular rosacea); erysipelas; erythrasma; ecthyma; ecthyma gangrenosum; impetigo; paronychia; cellulitis; folliculitis (including hot tub folliculitis); furunculosis; carbunculosis; staphylococcal scalded skin syndrome; surgical scarlet fever; streptococcal perianal disease; streptococcal toxic shock syndr ome; pitted keratolysis; trichomycosis axillaris; pyoderma; external canal ear infections; green nail syndrome; spirochetes; necrotizing fasciitis; Mycobacterial skin infections (such as lupus vulgaris, scrofuloderma, warty tuberculosis, tuberculides, erythema nodosum, erythema induratum, cutaneous manifestations of tuberculoid leprosy or lepromatous leprosy, erythema nodosum leprosum, cutaneous M. kansasii, M. malmoense, M. szulgai, M. simiae, M. gordonae, M. haemophilum, M. avium, M. intracellulare, M. chelonae (including M. abscessus) or M. fortuitum infections, swimming pool (or fish tank) granuloma, lymphadenitis and Buruli ulcer (Bairnsdale ulcer, Searles' ulcer, Kakerifu ulcer or Toro ulcer)); atopic eczma with staphylococcal carriage; as well as infected eczma, burns, abrasions and skin wounds.

Particular fungal conditions that may be treated by topical pharmaceutical compositions and combination products according to the present invention also include include the skin- and membrane-related conditions disclosed hereinbefore, as well as: candidiasis; sporotrichosis; ringworm (e.g. tinea pedis, tinea cruris, tinea capitis, tinea unguium or tinea corporis); tinea versicolor; and infections with Trichophyton, Microsporum, Epidermophyton or Pityrosporum ovale (Malassezia furfur) fungi.

In addition to the above, the topical compositions and combination products according to the present invention may be used to effect clearance (e.g. prophylactic clearance) of:
(a) Staphylococci (e.g. MRSA);
(b) Propionibacteria, such as Propionibacterium acnes; or
(c) fungi (such as Candida albicans, Cryptococcus neoformans, Histoplasma capsulatum, Epidermophyton floccosum, Malassezia (e.g. Malassezia furfur) or, particularly, Trichophyton (e.g. Trichophyton violaceum, Trichophyton mentagrophytes or, particularly, Trichophyton rubrum), from the skin or membranes of a patient in need of such clearance.

In the case of Staphylococci the clearance may be effected particularly from the skin (e.g. before surgery or insertion of medical equipment such as i.v. needles, catheters and tracheostomy or feeding tubes), nose (e.g. anterior nares), wounds or atopic eczma (atopic dermatitis).

Thus, according to a further aspect of the invention, there is provided a method for treating any of the above-mentioned conditions and infections, or of effecting clearance of microorganisms as described above, the method comprising administering to a patient in need thereof an effective amount of a topical composition according to the first aspect of the invention, or a combination product according to the second aspect of the invention.

Similarly, there is provided a topical composition according to the first aspect of the invention, or a combination product according to the second aspect of the invention for use in the treatment of any of the above-mentioned conditions and infections, or in effecting clearance of microorganisms as described above.

For the avoidance of doubt, as used herein, the term “treatment” includes therapeutic and/or prophylactic treatment.

The microorganisms killed by application of the topical composition or combination product may be clinically latent. Thus, the invention also encompasses a method of killing clinically latent microorganisms in a mammal infected with such latent microorganisms, the method comprising administering to said mammal a microbicidally effective amount of a topical composition according to the first aspect of the invention, or a combination product according to the second aspect of the invention.

As well as having activity against fungi and bacteria, compounds of formula I may also have activity against other organisms, such as protozoa. Therefore, according to further aspects of the invention, there is provided:
(i) a method of treating a topical protozoal disease, said method comprising administering to a patient in need thereof an effective amount of a topical pharmaceeutical composition according to the first aspect of the invention;
(ii) a topical pharmaceeutical composition according to the first aspect of the invention for use in the treatment of a topical protozoal disease.

When used herein, the term “topical protozoal disease” includes references to Leishmaniasis and infections with Trichomonas vaginalis (such as trichomoniasis).

Compounds of formula I may be prepared in accordance with techniques known to those skilled in the art, for example as described hereinafter.

Process that may be employed for the preparation of a compound of formula I include:
(a) for compounds of formula I in which X represents —C(R^{8a})(R^{8b})—C(R^{8e})(R^{8d})—, reaction of a compound of foimula II,

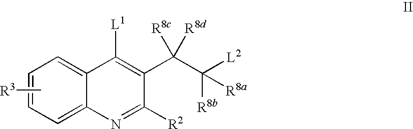

wherein L¹ and L² independently represent a suitable leaving group (e.g. halo) and R², R³, R^{8a}, R^{8b}, R^{8c} and R^{8d} are as hereinbefore defined, with a compound of formula III,

R¹—NH₂ III

wherein R¹ is as hereinbefore defined, for example under conditions known to those skilled in the art (e.g. by reaction at elevated temperature (such as 70 to 225° C.) and/or pressure (i.e. above 1 atmosphere) in the presence of a suitable organic solvent, such as a C₁₋₄ alcohol (e.g. ethanol or n-butanol) (for example, the reaction may be performed by reaction of the compound of formula II with between 1 and 3 equivalents (e.g. from 1.5 to 2 equivalents) of the compound of formula III at elevated temperature (e.g. above 120° C., such as from 150 to 200° C. or, particularly, from 175 to 185° C. (e.g. 180° C.)), wherein the reaction mixture is optionally heated by use of microwaves, in the presence of a suitable high-boiling solvent (e.g. an alkylene glycol, such as ethylene glycol) or, when the compound of formula III is liquid at the reaction temperature, in the presence of excess compound of formula III); or
(b) for compounds of formula I in which X represents —C(R^{8e})═C(R^{8f})—, dehydrogenation of a corresponding compound of formula I in which X represents —C(H)(R^{8a})—C(H)(R^{8c})—, for example under conditions known to those skilled in the art (such as reaction at elevated temperature (e.g. from 70 to 225° C.) in the presence of a (de)hydrogenation catalyst (e.g. palladium supported on carbon) and a suitable, reaction-inert solvent (e.g. diphenylether)).

In the formation of compounds of formula I in which X represents —C(R^{8a})(R^{8b})—C(R^{8c})(R^{8d})— (e.g. as outlined at (a) above), elimination of extrane (e.g. atmospheric oxygen), may be utilised in order to minimise the formation of corresponding compounds of formula I in which X represents —C(R^{8e})═C(R^{8f})—. This may be effected, for example, by degassing reaction solvents and/or reagents, or by use of an antioxidant (e.g. at a low level, such as 0.5 mol. %) such as butylated hydroxytoluene (“BHT”).

Compounds of formula II in which L¹ and L² both represent halo may be prepared according to methods known to those skilled in the art, for example by reaction of a corresponding compound of formula IV,

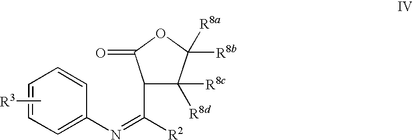

wherein R², R³, R^{8a}, R^{8b}, R^{8c} and R^{8d} are as hereinbefore defined, with a combined dehydrating/halogenating agent (e.g. P(O)Cl₃), for example under conditions know to those skilled in the art (e.g. at elevated temperature, optionally in the presence of a suitable organic solvent). For example, the reaction may be performed by reaction at elevated temperature (e.g. from 75 to 120° C., such as from 90 to 100° C.) of the compound of formula IV with from 1 to 5 (e.g. 2) equivalents of P(O)Cl₃, optionally (and preferably) in the presence of a suitable solvent (e.g. acetonitrile or, particularly, toluene).

Compounds of formula IV may be prepared by reaction of a corresponding compound of formula V,

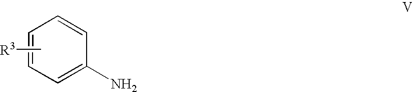

wherein R³ is as hereinbefore defined, with a compound of formula VI,

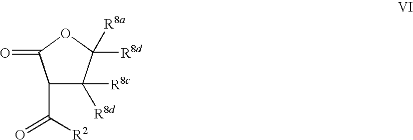

R², R^{8a}, R^{8b}, R^{8c} and R^{8d} are as hereinbefore defined, for example under conditions know to those skilled in the art (e.g. at elevated temperature, such as from 100 to 180° C.). For example, the reaction may be performed by reaction at elevated temperature (e.g. from 75 to 120° C., such as from 100 to 118° C.) of the compound of formula V with from 1 to 1.5 equivalents (e.g. 1 or 1.1 equivalents) of the compound of formula VI in the presence of a suitable solvent (e.g. a high-boiling, water-immiscible hydrocarbon, such as toluene) and optionally in the presence of a suitable catalyst (e.g. an acid, such as acetic acid or, particularly, an acidic polymer resin (ion exchange resin), such as a polysulfonated polymer of styrene or copolymer of styrene and divinylbenzene (e.g. Amberlyst 15)). In this instance, the reaction may be performed in the presence of a dehydrating agent (such as molecular sieves) or in such a way that water generated by the condensation reaction is removed whilst the reaction is in progress (e.g. by use of a water-immiscible solvent such as toluene and a Dean-Stark apparatus, as known to those skilled in the art).

Compounds of formulae III, V and VI are either commercially available, are known in the literature, or may be obtained by analogy with the processes described herein, or by conventional synthetic procedures, in accordance with standard techniques, from readily available starting materials using appropriate reagents and reaction conditions.

Substituents on alkyl, alkenyl, alkynyl, cycloalkyl, cycloalkenyl, aryl and heterocyclic groups in compounds of formulae I, II, III, IV, V and VI may be introduced and/or interconverted using techniques well known to those skilled in the art by way of standard functional groups interconversions, in accordance with standard techniques, from readily available starting materials using appropriate reagents and reaction conditions. For example, hydroxy may be converted to alkoxy, phenyl may be halogenated to give halophenyl, halo may be displaced by cyano, etc.

Compounds of formula I may be isolated from their reaction mixtures using conventional techniques. For example, compounds of formula I may be isolated by conversion to an acid (e.g. hydrochloric acid) salt (e.g. by way of addition of acid to the crude product) and then recrystallisation of the salt from a suitable solvent (e.g. methanol or, particularly, ethanol). Alternatively, the salt can simply be washed with or slurried in the presence such a suitable solvent in order to isolate the pure acid salt of the compound of formula I.

In accordance with the present invention, pharmaceutically acceptable derivatives of compounds of formula I also include “protected” derivatives, and/or compounds that act as prodrugs, of compounds of formula I.

Compounds of formula I may exhibit tautomerism. All tautomeric forms and mixtures thereof are included within the scope of the invention.

Compounds of formula I may also contain one or more asymmetric carbon atoms and may therefore exhibit optical and/or diastereoisomerism. Diastereoisomers may be separated using conventional techniques, e.g. chromatography. The various stereoisomers may be isolated by separation of a racemic or other mixture of the compounds using conventional, e.g. HPLC techniques. Alternatively the desired optical isomers may be made by reaction of the appropriate optically active starting materials under conditions which will not cause racemisation or epimerisation, or by derivatisation, for example with a homochiral acid followed by separation of the diastereomeric derivatives by conventional means (e.g. HPLC, chromatography over silica). All stereoisomers are included within the scope of the invention.

It will be appreciated by those skilled in the art that in the processes described above and hereinafter the functional groups of intermediate compounds may need to be protected by protecting groups.

Functional groups that it is desirable to protect include hydroxy, amino and carboxylic acid. Suitable protecting groups for hydroxy include optionally substituted and/or unsaturated alkyl groups (e.g. methyl, allyl, benzyl or tert-butyl), trialkylsilyl or diarylalkylsilyl groups (e.g. t-butyldimethylsilyl, t-butyldiphenylsilyl or trimethylsilyl) and tetrahydropyranyl. Suitable protecting groups for carboxylic acid include C₁₋₆ alkyl or benzyl esters.

The protection and deprotection of functional groups may take place before or after coupling, or before or after any other reaction in the above-mentioned schemes.

Protecting groups may be removed in accordance with techniques that are well known to those skilled in the art and as described hereinafter.

Persons skilled in the art will appreciate that, in order to obtain compounds of formula I in an alternative, and, on some occasions, more convenient, manner, the individual process steps mentioned hereinbefore may be performed in a different order, and/or the individual reactions may be performed at a different stage in the overall route (i.e. substituents may be added to and/or chemical transformations performed upon, different intermediates to those mentioned hereinbefore in conjunction with a particular reaction). This may negate, or render necessary, the need for protecting groups.

The type of chemistry involved will dictate the need, and type, of protecting groups as well as the sequence for accomplishing the synthesis.

The use of protecting groups is described in “Protective Groups in Organic Chemistry”, edited by J W F McOmie, Plenum Press (1973), and “Protective Groups in Organic Synthesis”, 3^{rd} edition, T. W. Greene & P. G. M. Wutz, Wiley-Interscience (1999).

Protected derivatives of compounds of formula I may be converted chemically to compounds of the invention using standard deprotection techniques (e.g. hydrogenation). The skilled person will also appreciate that certain compounds of formula I may also be referred to as being “protected derivatives” of other compounds of formula I.

Those skilled in the art will also appreciate that certain compounds of formula I will be useful as intermediates in the synthesis of certain other compounds of formula I.

Topical pharmaceutical compositions and combination products according to the present invention have the advantage that they may be used to kill clinically latent microorganisms. Further, in treating microbial infections, topical pharmaceutical compositions and combination products according to the present invention may possess the further advantage that they allow for a shorter period of therapy, thus increasing patient compliance (through, for example, the need to take fewer or smaller doses of antimicrobial agents) and/or minimising the risk of generating sub-populations of microorganisms that are (genetically) resistant to conventional antimicrobial agents.

Additionally, topical pharmaceutical compositions and combination products according to the invention may have the advantage that they may be more stable than, more efficacious than, be less toxic than, have a broader range of activity than, be more potent than, produce fewer side effects than, or have other useful physical or pharmacological properties over other antimicrobial compositions known in the prior art.

Biological Tests

Test procedures that may be employed to determine the biological (e.g. bactericidal or antibacterial) activity of the (compositions comprising) compounds of formula I include those known to persons skilled in the art for determining:
(a) bactericidal activity against stationary-phase or “persister” bacteria (i.e. “clinically latent” bacteria); and
(b) antibacterial activity against log phase bacteria.

In relation to (b) above, methods for determining activity against log phase bacteria include a determination, under standard conditions (i.e. conditions known to those skilled in the art, such as those descried in WO 2005/014585, the disclosures of which document are hereby incorporated by reference), of Minimum Inhibitory Concentration (“MIC”) or Minimum Bactericidal Concentration (“MBC”) for a test compound.

In relation to (a) above, methods for determining activity against clinically latent bacteria include a determination, under conditions known to those skilled in the art (such as those described in Nature Reviews, Drug Discovery 1, 895-910 (2002), the disclosures of which are hereby incorporated by reference), of Minimum Stationary-cidal Concentration (“MSC”) or Minimum Dormicidal Concentration (“MDC”) for a test compound. Specific examples of such methods are described below.

Protocol for Pyogenic Bacteria

Bacterial Strains

Unless otherwise specified, the strains used for screening were those shown in the following table.

| | | |
|---|---|---|
| Staphylococcus aureus (Oxford) | Gram positive | Reference strain |
| Escherichia coli K12 | Gram negative | Reference strain |
| Enterococcus | Gram positive | Clinical isolate |
| Pseudomonas | Gram negative | Clinical isolate |
| Methicillin resistant S. aureus | Gram positive | Clinical isolate |
| (MRSA) | | |
| Klebsiella aerogrenes | Gram negative | Clinical isolate |
| E. coli | Gram negative | Clinical isolate |
| Streptococcus pneumoniae | Gram positive | Reference strain |
| Streptococcus pyogenes Group A | Gram positive | Reference strain |
| Streptococci | | |
| Group B streptococci (Streptococcus | Gram positive | Reference strain |
| agalactiae) | | |
| Streptococcus viridans | Gram positive | Reference strain |
| Haemophilus influenzae | Gram negative | Reference strain |
| Coagulase negative staphylococcus | Gram positive | Reference strain |
| (CNS) | | |
| Propionibacterium acnes | Gram positive | Reference strain |
| | | (NCTC 737) |

Growth of Bacteria

The bacteria (except for streptococci, H. influenzae and P. acnes) were grown in 10 mL of nutrient broth (No. 2 (Oxoid)) overnight at 37° C., with continuous shaking at 120 rpm. Streptococci and H. influenzae were grown overnight in Todd-Hewitt broth (Sigma) without shaking. P. acnes was grown overnight in 10 mL of nutrient broth without shaking. The overnight cultures were diluted (1000×) in 100 mL of growth medium and then incubated with or without shaking for 10 days. Viability of the bacteria was estimated by colony forming unit (CFU) counts at 2 hours intervals at the first 24 hours and at 12-24 hours afterwards. From serial 10-fold dilutions of the experimental cultures, 100 μL samples were added to triplicate plates of nutrient agar plates (Oxoid) and blood agar plates (Oxoid). Colony forming units (CFU) were counted after incubation of the plates at 37° C. for 24 hours. CFU counts of P. acnes were estimated after the plates were incubated under anaerobic conditions for 48 hours.

Log-phase cultures: The above-described overnight cultures were diluted (1000×) with iso-sensitest broth. The cultures were then incubated at 37° C. with shaking for 1-2 hours to reach log CFU 6, except for streptococci, H. influenzae and P. acnes, which were incubated at 37° C. without shaking. These cultures were served as log-phase cultures.

Stationary phase cultures: Cultures incubated for more than 24 hours are in stationary phase. For drug screening, 5-6 day old stationary phase cultures are used. The cultures were diluted with phosphate buffered saline to log 6, which were used to incubate with testing compounds.

Measurements of Bactericidal Activity Against Log-Phase Cultures

Different concentrations of each test compound were incubated with the log-phase cultures in 96 well plates for various periods of time (2, 4, 6, 12, 24 hours). Bactericidal activity was then examined by taking a spectrophotometer reading (using a plate reader) of the resulting cultures, as well as by CFU counts as described above.

Measurements of Bactericidal Activity Against Stationary-Phase Cultures

Different concentrations of each test compound were incubated with stationary phase cultures (5-6 day cultures) in 96 well plates for 24 or 48 hours. Bactericidal activity was then determined by taking CFU counts of the resulting cultures, as described above.

Measurements of Bactericidal Activity Against Persistent Bacteria

An antibiotic (e.g. gentamicin) was added to 5-6 day stationary-phase cultures to the final concentration of 50 to 100 μg/mL for 24 hours. After 24 hours of antibiotic treatment, the cells are washed 3 times with phosphate buffered saline

(PBS), and then resuspended in PBS. The surviving bacterial cells are used as persisters. Viability is estimated by CFU counts. The persisters were then used in measurements of bactericidal activity for test compounds.

Different concentrations of each test compound were incubated with the (persister) cell suspension in 96 well plates for various periods of time (24 and 48 hours). Bactericidal activity was then determined by taking CFU counts of the resulting cultures, as described above.

Protocol for M. tuberculosis

Growth of M. tuberculosis

M. tuberculosis H37Rv was grown in 10 mL of Middlebrook 7H9 broth containing 0.05% Tween 80 supplemented with 10% ADC without disturbing for up to 100 days. In order to obtain evenly dispersed cultures prior to experimental treatment, clumps in the cultures were broken up by vortexing the cultures in the presence of 2 mm glass beads (Philip Harris Scientific, Staffordshire, UK) for 2 minutes, followed by sonication in a water bath sonicator (Branson Ultrasonic B. V.) for 5 minutes. The numbers of viable M. tuberculosis in the cultures were determined by colony forming unit (CFU) counts on Middlebrook 7H11 agar.

Serials of 10-fold dilutions of the cultures are made in Middlebrook 7H9 broth with 0.05% (v/v) Tween 80 but without ADC. Then, 100 μL of samples was added to one-third segments of the agar plates in duplicate. The plates were incubated in polythene bags for 3 weeks at 37° C.

Measurements of Bactericidal Activity Against Log-Phase Cultures

Different concentrations of each test compound were incubated with log-phase cultures (4 day cultures) for various time periods (4, 8, 16, 24 days). Bactericidal activity was then determined by taking CFU counts of the resulting cultures, as described above.

Measurements of Bactericidal Activity Against Stationary-Phase Cultures and Persistent Bacteria

Model 1—Stationary-phase cultures. Different concentrations of each test compound were incubated with the sonicated 100-day cultures, each concentration to a separate 10 mL culture. After incubation for 5 days, counts of viable CFU were determined by inoculating a pair of 7H11 plates with 100 μL of 10-fold serial dilutions of the resulting cultures.

Model 2—Persistent bacteria selected by rifampicin. Rifampicin (100 mg/L) was added to each of a set of sonicated 100-day cultures, which cultures were then incubated for 5 days. After the first day of incubation, no colonies could be obtained on plates inoculated from the culture. After washing twice with PBS by centrifugation, fresh (and rifampicin-free) 7H9 medium was added to make up the volume to 10 mL and the test compound was added in the same concentrations as in model 1. After further incubation for 7 days, CFU counts were determined by inoculating 1 mL from each container onto a 7H11 plate. These plates were then incubated for 2 weeks and the very small colonies were counted and marked. After a further 2 weeks of incubation, any additional unmarked colonies (i.e. those that grew slowly) were added to the counts. Control studies have shown that plate counts begin to yield colonies on subculture after about 4 days of incubation of the rifampicin-free cultures.

Model 3. The procedure is similar to model 2, but only different concentrations of the test compound was added to the 100-day culture at three days after the rifampicin treatment. At the end of the 7-day incubation period (4 days with candidate drugs plus rifampicin), all cultures were washed, replacing with medium free of test compound, and then were incubated for a further 7 days before CFU counts were determined.

Protocol for Candida albicans

Candida albicans, a clinical isolate was used. The strain was grown in 10 mL of

Potato dextrose broth medium (Sigma-Aldrich) at 24° C., with continuous shaking at 120 rpm for 24 hours. Then, 1 mL of the culture was inoculated in 100 mL of fresh broth medium, which was incubated at the same conditions for 6 days.

Log-phase cultures: The above-described 24 hour culture was diluted (100×) with potato glucose broth medium. The cultures were then incubated at 24° C. with shaking for 20-24 hours served as log-phase cultures. The log phase cultures were diluted with fresh broth medium to CFU log 6, which were used to test the activities of compounds.

Stationary phase cultures: For drug screening, 5-6 day old stationary phase cultures were used. The stationary phase cultures were diluted with phosphate buffered saline to CFU log 6, which were used to examine the activities of test compounds.

Measurements of Activity Against Log-Phase Cultures

Different concentrations of each test compound were incubated with the log-phase cultures in 96 well plates for various periods of time (2, 4, 6, 12, 24 hours). The activity of drugs was then examined by taking a spectrophotometer reading (using a plate reader) of the resulting cultures, as well as by CFU counts as described above.

Measurements of Activity Against Stationary-Phase Cultures

Different concentrations of each test compound were incubated with stationary phase cultures (5-6 day cultures) in 96 well plates for 24 or 48 hours. The activity was then determined by taking CFU counts of the resulting cultures, as described above.

Skin (Topical) Models

In addition to in vitro testing against stationary- and log-phase bacteria, compounds of formula I may also be tested in various in vivo models, including those known to those skilled in the art. For example, for determination of compound activity against bacteria in or on the skin, protocols that may be followed include those described in Antimicrobial Agents and Chemotherapy 49(8), 3435-41 (2005), as well as the following.

Mouse Superficial Skin Bacterial Model (Intact Skin)

ICR or BALB/c mice aged 6-8 weeks were obtained from Harlan UK. The mice were anaesthetized by intraperitoneal injection of 200 μL of ketamine hydrochloride/xylazine solution. Fur on the back of the mouse was removed using an electrical clipper. A 2 cm² (2 cm×1 cm) area of skin was marked with a marker pen. The marked skin area was swabbed 3 times using a disposable swab in order to examine the bacterial numbers on the skin. The bacteria on the swab were spread on blood agar plates (Oxoid™).

Log-phase or stationary phase bacterial or yeast cultures were used. The cultures were concentrated by centrifugation to obtain 10⁹ to 10¹⁰ CFU/mL. The cell pellet was resuspended with nutrient broth or PBS and glycerol (50%). 15-20 μL of the cell suspension was added to the skin area (2 cm²) which gave 10⁶⁻⁷ CFU on the skin. The skin was allowed to dry for about 15 min. Solutions (or more viscous compositions, such as aqueous gels) of test compound at different concentrations were applied on the skin area for different periods of time.

Bacterial or yeast numbers on the skin were estimated as follows: After the mouse was euthanised, the skin at the marked area was cut and added to a 2 mL tube containing 1 mL water and glass beads (1 mm). The skin was homogenised using a reciprocal shaker (Hybaid Ltd, UK) for 45 seconds (speed setting 6.5) or votexing for 1 min. Residual test compound was removed by washing 3 times with water or PBS (if the test compound precipitated in the buffer system, water alone was used for washing). CFU counts were performed after a serial of 10 fold dilution of the homogenates. 100 μL samples were added to one third of blood agar plates (Oxoid™) in duplicate. Colony forming units (CFU) were then counted using aCoLye (a colony counter) after incubation of the bacterial plates at 37° C. for 24 hours or yeast plates at 24° C. for 48 hours.

Mouse Superficial Skin Infection Model (Tape-Stripping Infection Model)

ICR or BALB/c mice aged 6-8 weeks were obtained from Harlan UK. The mice were anaesthetized by intraperitoneal injection of 200 μL of ketamine hydrochloride/xylazine solution. The fur of the mice on the back was removed by electric clipper. An area of 2 cm² skin was tape-stripped using autoclave tape. The skin was stripped 10 times in succession. After this procedure, the skin became visibly damaged and was characterized by reddening and glistening but no regular bleeding. Buprenorphine was given during the anaesthetic period and every 12 hours for up to 3 days to reduce prolonged pain. After stripping of the skin, a bacterial infection was initiated by placing 10 μL of bacterial cell suspension containing 10⁷ cells from overnight or stationary phase cultures on the damaged skin area. At 0 and 4 hours after infection, 3 mice were killed to estimate the CFU counts on the skin. After 24 hours, solutions (or more viscous compositions, such as aqueous gels) of test compound at different concentrations were applied on the skin area for different periods of time. The experiments were terminated 18 h after the last topical treatment.

Bacterial numbers of the wounds were estimated as follows: After the mouse was been euthanised, the wounds, approximately 2 cm² were cut and added to a 2 mL tube containing 1 mL water and glass beads (1 mm). The skin was homogenised using a reciprocal shaker (Hybaid Ltd, UK) for 45 seconds (speed setting 6.5). Residual test compound was removed by washing 3 times with water. CFU counts were performed after a serial of 10 fold dilution of the homogenates. 100 μL samples were added to one third of blood agar plates (Oxoid™) in duplicate. Colony forming units (CFU) were counted using aCoLye (a colony counter) after incubation of the plates at 37° C. for 24 hours.

The invention is illustrated, but in no way limited, by the following examples and by reference to the figures, which present data relating, inter alia, to the biological studies described above.

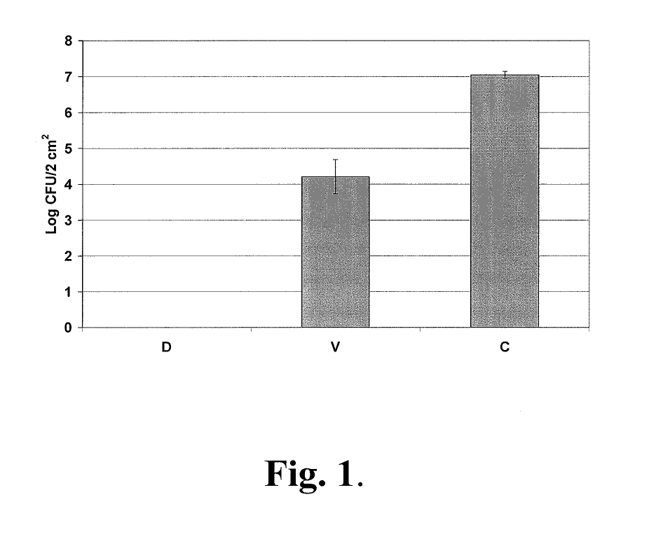

FIG. 1 illustrates the effect of compound 4-methyl-1-(2-phenylethyl)-8-phenoxy-2,3-dihydro-1H-pyrrolo-[3,2-c]quinoline hydrochloride (the compound of Preparative Example 9) against stationary phase Candida albicans on intact mouse skin. The test compound was applied to the mouse skin as an aqueous gel formulated as described in Example 2 below (see Formulation 10).

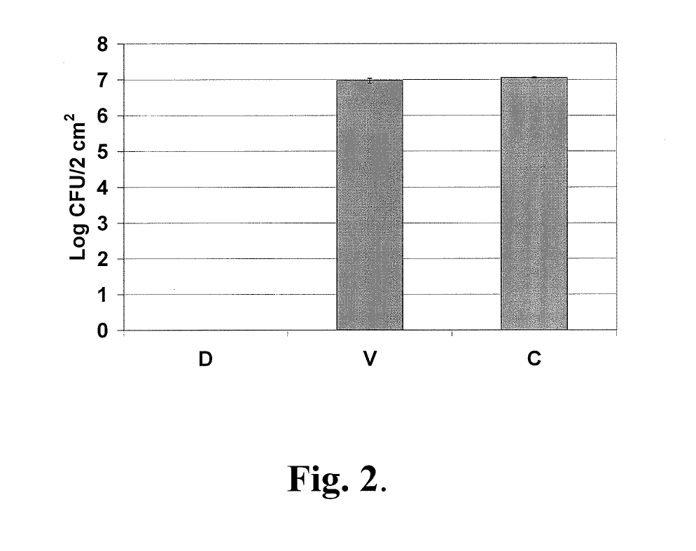

FIG. 2 illustrates the effect of compound 4-methyl-1-(2-phenylethyl)-8-phenoxy-2,3-dihydro-1H-pyrrolo-[3,2-c]quinoline hydrochloride (the compound of Preparative Example 9) against stationary phase Staphylococcus aureus on intact mouse skin. The test compound was applied to the mouse skin as an aqueous gel formulated as described in Example 2 below (see Formulation 10).

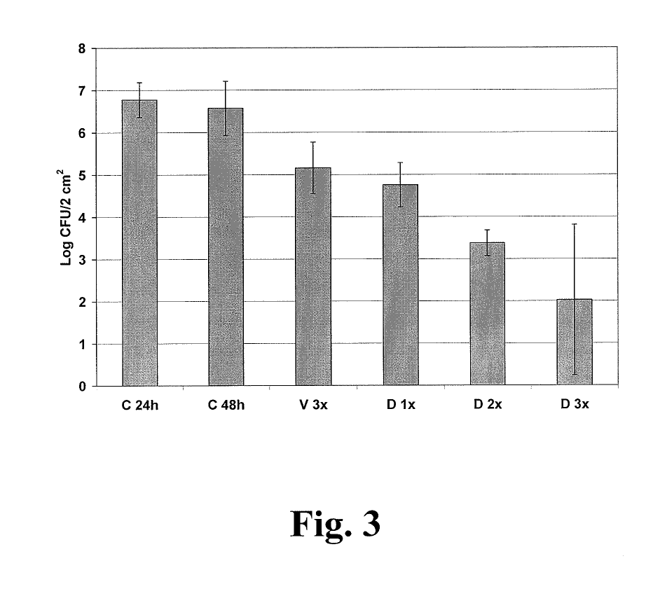

FIG. 3 illustrates the effect of compound 4-methyl-1-(2-phenylethyl)-8-phenoxy-2,3-dihydro-1H-pyrrolo-[3,2-c]quinoline hydrochloride (the compound of Preparative Example 9) against stationary phase Staphylococcus aureus on infected mouse skin (tape stripping model described above). The test compound was applied (one, two or three times) to the mouse skin as an aqueous gel formulated as described in Example 2 below (see Formulation 10).

Key
HT61: 4-methyl-1-(2-phenylethyl)-8-phenoxy-2,3-dihydro-1H-pyrrolo-[3,2-c]quinoline hydrochloride
C: control (no treatment)
D: aqueous gel formulation containing test compound (see Example 2 below, Formulation 10)
V: aqueous gel formulation lacking test compound (see Example 2 below, Formulation 9(P)).
1×: single application of the formulation
2×: two applications of the formulation (3 hour time interval between each application)
3×: three applications of the formulation (3 hour time interval between each application)

### EXAMPLES

General Experimental Procedure

Analytical LC-MS data were obtained using either Method A or Method B as indicated.

Method A: A Hewlett Packard HP1100 LC system using a 30×4.6 mm 3 micron Phenomenex Luna C18 column eluting at 2 mL/min with a gradient (5-95% over 4 minutes) of MeCN/water (+0.1% formic acid). Detection by mass spectrometry used a Micromass Platform LC quadrupole instrument in both positive and negative electrospray mode. Detection was also performed using a Sedex 65 evaporative light scattering detector and an HP1100 Diode array detector.

Method B: A Hewlett Packard 1050 LC system using a 100×3 mm 5 micron Higgins Clipeus C18 column eluting at 2 mL/min with a gradient (5 to 95% over 15 minutes) of MeCN/water (+0.1% formic acid). Detection by mass spectrometry used a Finnigan TSQ700 triple quadrupole instrument in positive electrospray mode. Detection was also performed by UV absorption at 254 nm.

Starting Materials

The following, commercially available compounds may be employed in the syntheses described below.

List 1

| | |
|---|---|
| 2,4-Dimethoxyaniline. | 4-Chloroaniline. |
| 4-Methoxyaniline. | 4-(Morpholin-4-yl)aniline. |
| 4-Phenoxyaniline. | Ethyl 4-aminophenylacetate. |
| 2-Phenoxyaniline. | Methyl 4-aminobenzoate. |
| 4-Ethoxyaniline. | 4-Cyanoaniline. |
| 4-Trifluoromethoxyaniline. | 4-Hydroxyaniline. |
| 4-(Piperidin-1-yl)aniline. | |

List 2

| | |
|---|---|
| Cyclopropylamine. | Aniline. |
| 3-Phenoxyaniline. | 4-Phenoxyaniline. |
| 4-(2-Dimethylaminoethoxy)aniline. | 4-(Pyridin-3-yloxy)aniline. |
| 1-Benzyl-piperidin-4-ylamine. | Indan-2-ylamine. |
| Benzylamine. | 2-Phenylethylamine. |
| 2-Phenoxyethylamine. | 4-Hydroxyaniline. |
| 4-Methoxyaniline. | 3-Hydroxyaniline. |
| 3-Hydroxy-5-methylaniline. | 5-Amino-2-phenoxypyridine. |
| Indan-1-ylamine. | 3,4-Methylenedioxaniline. |
| 3-Methylbutylamine. | 5-Amino-2-methoxypyridine. |
| 4-iso-Propylaniline. | Cyclopropylmethylamine. |
| 4-Bromo-3-fluoroaniline. | Benzodioxan-2-ylmethylamine. |
| 1-Amino-1,2,3,4- | Cyclohexylamine. |
| tetrahydronaphthalene. | 2-Methylbenzylamine. |
| 1-Phenylethylamine. | 4-(4-Fluorophenoxy)aniline. |
| 4-(Piperidin-1-yl)aniline. | 2-Pyridylmethylamine. |
| 2-(3-Pyridyl)ethylamine. | 1-(3-Aminopropyl)pyrrolidin- |
| (5-Methylpyrazin-2-yl)methylamine. | 2-one. |
| 2-(2-Pyridyl)ethylamine. | Ethyl 3-aminopropionate |
| Ethyl 4-aminobutanoate. | Methyl 3-aminopropionate. |
| Methyl 4-aminobutanoate. | Ethyl aminoacetate. |
| 4-Amino-1-methylpiperidine. | 1-Benzyl-3-aminopyrrolidine. |
| 3-Methoxypropylamine. | Tetrahydrofuran-2-ylmethylamine. |
| 2-(4-Chlorophenyl)ethylamine. | 2-(4-Methoxyphenyl)ethylamine. |
| 2-Phenylpropylamine. | |

Preparations

Preparation 1

The compounds listed below were prepared by the following general method.

The relevant aniline (0.05 mol; see List 1) and 2-acetyl-5-butyrolactone (0.05 mol) were heated to 120° C. for one hour, and then heated to 160° C. for two hours. After cooling to room temperature, phosphoryl chloride (50 mL) was added and the mixture heated at reflux for one hour. After cooling to room temperature again, the mixture was poured onto crushed ice (100 g) and neutralised with sodium carbonate (added as a solid). The resulting oily product was extracted into dichloromethane (50 mL) and the organic solution washed with water (25 mL), then brine (25 mL) and dried with anhydrous magnesium sulphate. Filtration and evaporation gave a brown solid, recrystallisation of which from ethanol gave the target substituted 4-chloro-3-(2-chloroethyl)-2-methylquinoline as a colourless or off-white solid.

### (a) 4-Chloro-3-(2-chloroethyl)-6,8-dimethoxy-2-methylquinoline

LCMS (Method A): Rt=3.17 min, m/z=300.06 [M+H]⁺; C₁₄H₁₅Cl₂NO₂, Mono-isotopic mass=299.1.

### (b) 4-Chloro-3-(2-chloroethyl)-6-methoxy-2-methylquinoline

LCMS (Method A): Rt=3.16 min, m/z=269.98 [M+H]⁺; C₁₃H₁₃Cl₂NO, Mono-isotopic mass=269.0.

### (c) 4-Chloro-3-(2-chloroethyl)-2-methyl-6-phenoxyquinoline

LCMS (Method A): Rt=4.38 min, m/z=332.00 [M+H]⁺; C₁₈H₁₁₅Cl₂NO, Mono-isotopic mass=331.05.

### (d) 4-Chloro-3-(2-chloroethyl)-2-methyl-8-phenoxyquinoline

LCMS (Method A): Rt=4.27 min, m/z=332.01 [M+H]⁺; C₁₈H₁₅Cl₂NO, Mono-isotopic mass=331.05.

### (e) 4-Chloro-3-(2-chloroethyl)-6-ethoxy-2-methylquinoline

LCMS (Method A): Rt=3.54 min, m/z=284.16 [M+H]⁺; C₁₄H₁₅Cl₂NO, Mono-isotopic mass=283.05.

### (f) 4-Chloro-3-(2-chloroethyl)-2-methyl-6-(morpholin-4-yl)quinoline

¹H NMR (400 Mz, D₆DMSO) δ 7.82 (d, J=9.3 Hz, 1H), 7.65 (dd, J=9.3, 2.7 Hz 1H), 7.23 (d, J=2.7 Hz 1H), 3.88 (t, J=6.7 Hz, 2H), 3.79 (m, 4H), 3.41 (t, J=6.7 Hz, 2H), 3.28 (m, 4H), 2.70 (s, 3H).

### (g) 4-Chloro-3-(2-chloroethyl)-2-methyl-6-trifluoromethoxyquinoline

LCMS (method A): Rt=4.39 min, m/z=323.89 [M+H]⁺; C₁₃H₁₀Cl₂F₃NO, Mono-isotopic mass=323.01.

### (h) 4-Chloro-3-(2-chloroethyl)-2-methylquinoline

LCMS (method A): Rt=3.14 min, m/z=240.13 [M+H]⁺; C₁₂H₁₁Cl₂N, Mono-isotopic mass=239.03.

### (i) 4-Chloro-3-(2-chloroethyl)-2,8-dimethylquinoline

LCMS (method A): Rt=4.53 min, m/z=253.98 [M+H]⁺; C₁₃H₁₃Cl₂N, Mono-isotopic mass=253.04.

### (j) 4-Chloro-3-(2-chloroethyl)-2-methyl-6-(piperidin-1-yl)quinoline

Used directly without purification.

### (k) 3-(2-Chloroethyl)-4,6-dichloro-2-methylquinoline

¹H NMR (400 Mz, D₆DMSO) δ 8.13 (d, J=2.2 Hz, 1H), 8.00 (d, J=9.0 Hz, 1H), 7.82 (dd, J=9.0 Hz, 2.2 Hz, 1H), 3.92 (t, J=7.6 Hz, 2H), 3.45 (t, J=7.6 Hz, 2H), 2.78 (s, 3H).

### (l) Methyl 4-chloro-3-(2-chloroethyl)-2-methylquinolin-6-carboxylate

LCMS (method A): Rt=3.80 min, m/z=298.05 [M+H]⁺; C₁₄H₁₃Cl₂NO₂, Mono-isotopic mass=297.03.

### (m) Ethyl 4-chloro-3-(2-chloroethyl)-2-methylquinolin-6-ylacetate

LCMS (method A): Rt=3.47 min, m/z=326.13 [M+H]⁺; C₁₆H₁₇Cl₂NO₂, Mono-isotopic mass=325.06.

### (n) 4-Chloro-3-(2-chloroethyl)-6-cyano-2-methylquinoline

LCMS (method A): Rt=3.69 min, m/z=264.95 [M+H]⁺; C₁₃H₁₀Cl₂N₂, Mono-isotopic mass=264.02.

### (o) 4-Chloro-3-(2-chloroethyl)-6-hydroxy-2-methylquinoline

Used directly without purification.

### (p) 6-Bromo-4-chloro-3-(2-chloroethyl)-2-methylquinoline

¹H NMR (400 Mz, CDCl₃) δ 8.98 (d, J=9.1 Hz, 1H), 8.54 (d, J=2.4 Hz, 1H), 8.07 (dd, J=9.1, 2.4 Hz, 1H), 3.92 (t, J=6.1 Hz, 2H), 3.59 (t, J=6.1 Hz, 2H), 3.31 (s, 3H).

Preparation 2

### 4-Chloro-3-(2-chloroethyl)-6-phenoxyquinoline

### (i) Sodium (2-oxodihydrofuran-3-ylidene)methoxide

A solution of ethyl formate (4.51 g) and γ-butyrolactone (5.0 g) in diethyl ether (50 mL) was added dropwise to a suspension of sodium hydride (60% oil dispersion, 2.56 g) in diethyl ether (100 mL) containing methanol (0.2 mL) at such a rate as to maintain gentle reflux. The resultant mixture was then stirred at room temperature for 48 hours. The mixture was evaporated to dryness and the residue was triturated with cyclohexane and the solid was collected by filtration to give the sub-title compound (7.46 g) as a white powder.

¹H NMR (400 Mz, D₂O) δ 8.35 (m, 1H), 4.25 (m, 2H), 2.70 (m, 2H).

### (ii) 3-[1-(4-Phenoxyphenylamino)methylidene]dihydrofuran-2-one

A mixture of sodium (2-oxodihydrofuran-3-ylidene)methoxide (1.0 g; see step (i) above) and 4-phenoxyaniline hydrochloride (1.62 g) in methanol (20 mL) was stirred and heated at reflux for 30 minutes. The resultant cooled mixture was poured into water and the solid was collected by filtration and washed with water and ethyl acetate. The resultant solid was purified by chromatography on silica eluting with a mixture of methanol and dichloromethane (0:100 increasing to 1:20) to give the sub-title compound (0.69 g) as a white solid.

¹H NMR (400 Mz, D₆-DMSO) δ 9.06 (d, J=13.7 Hz, 1H), 7.62 (dt, J=13.4, 2.1 Hz, 1H), 7.36 (m, 2H), 7.19 (d, J=8.7 Hz, 2H), 7.09 (m, 1H), 6.96 (m, 4H), 4.29 (t, J=7.6, 2H), 2.86 (td, J=7.6, 2.1 Hz, 2H).

### (iii) 4-Chloro-3-(2-chloroethyl)-6-phenoxyquinoline

A mixture of 3-[1-(4-phenoxyphenylamino)methylidene]dihydrofuran-2-one (0.2 g; see step (ii) above) and phosphorus oxychloride was stirred and heated at reflux for 30 minutes. The resultant cooled mixture was added carefully to water with ice cooling as required and extracted with diethyl ether. The organic phase was washed with aqueous brine solution, dried (MgSO₄) and filtered. The filtrate was evaporated to dryness and the residue was purified by chromatography eluting with a mixture of ethyl acetate and cyclohexane (1:3) to give the title compound (0.116 g) as a pale yellow oil.

¹H NMR (400 Mz, CDCl₃) δ 8.69 (s, 1H), 8.09 (d, J=9.2 Hz, 1H), 7.67 (d, J=2.6 Hz, 1H), 7.49 (dd, J=9.2, 2.6 Hz, 1H), 7.41 (m, 2H), 7.21 (m, 1H), 7.11 (m, 2H), 3.83 (t, J=7.1, 2H), 3.40 (t, J=7.1 Hz, 2H).

Preparation 3

### 3-[1-(4-Phenoxyphenylamino)ethylidene]dihydrofuran-2-one

Large Scale Process Outline.

| Step | | | |
|---|---|---|---|
| No. | Operation | Charges | |
| 1 | Set-up 10 L flange flask equipped with Dean-Stark | | |
| | separator (250 mL volume). | | |
| 2 | Charge to flask 4-phenoxyaniline (1 eq). | 1100 | g |
| 3 | Charge to flask 2-acetylbutyrolactone (1.1 eq). | 703 | mL |
| 4 | Charge to flask Amberlyst 15 ion exchange resin. | 110 | g |
| 5 | Charge to flask toluene (3 volumes) and agitate. | 3300 | mL |
| 6 | Heat reaction vessel to reflux. (Reflux temperature | | |
| | initially ~100° C. and as water is removed reflux | | |
| | temperature will increase to ~118° C.) | | |
| 7 | The reaction was stirred at reflux overnight. | | |
| | (When ~200 mL of water has collected in Dean-Stark | | |
| | trap remove sample concentrate in vacuo and analyse | | |
| | using ¹H NMR) | | |
| 8 | Reaction allowed to cool to 50° C. | | |
| 9 | The reaction mixture was filtered to remove the resin. | | |
| | Some precipitation of the product will occur in the | | |
| | Büchner flask. Addition of DCM will dissolve the | | |
| | material. Combine DCM mixture with toluene filtrate | | |
| | for concentration. | | |
| 10 | Concentrate the reaction mixture in vacuo. Divide the | | |
| | reaction mixture between several RB flasks. | | |
| 11 | Dry the crude solid in a vacuum oven overnight at | | |
| | 50° C. | | |
| 12 | The crude solid was recrystallised from absolute EtOH | 8800 | mL |
| | (8 volumes). (Dissolved at 65-70° C. and on cooling | | |
| | precipitated at ~50° C.) | | |

Preparation 4

### 4-Chloro-3-(2-chloroethyl)-2-methyl-6-phenoxyquinoline

Large Scale Process Outline.

| Step | | | |
|---|---|---|---|
| No. | Operation | Charges | |
| 1 | Set-up 10 L flange flask equipped with HCl | | |
| | scrubber. | | |
| 2 | Charge to flask 3-[1-(4-phenoxyphenylamino)- | 709 | g |
| | ethylidene]dihydrofuran-2-one (1 eq.; see | | |
| | Preparation 3 above). | | |
| 3 | Charge to flask toluene (5 volumes). | 3550 | mL |
| 4 | Charge to flask phosphorus oxychloride (2 eq). | 448 | mL |
| 5 | The reaction flask was heated to 70° C. and then | | |
| | gradually to 90° C. at which point a slow exotherm | | |
| | increased the reaction temperature to reflux. | | |
| 6 | The reaction temperature was maintained at 100° C. | | |
| | for 2 h, monitored for completion using LC analysis. | | |
| | Reaction deemed complete when <1% of 3-[1- | | |
| | (4-phenoxy-phenylamino)ethylidene]dihydrofuran- | | |
| | 2-one present. Longer stir out if >1%. | | |
| | NB Gas evolution observed at this stage. | | |
| 7 | The reaction was allowed to cool to 50° C. and | 3550 | mL |
| | added water dropwise initially (5 volumes). | | |
| | Reaction will exotherm and can be controlled by | | |
| | addition of water. NB Gas evolution observed at | | |
| | this stage. | | |
| 8 | The reaction mixture was neutralised by addition of | ~1150 | g |
| | potassium carbonate. | | |
| 9 | The reaction mixture was filtered to remove | | |
| | inorganic solids. | | |
| 10 | The toluene layer was separated, dried over MgSO₄ | | |
| | and concentrated in vacuo to yield the crude | | |
| | product. NB Concentrate in batches of 1 L and | | |
| | remove solid from the flask before concentrating | | |
| | any further toluene. | | |
| 11 | Dry the crude solid in a vacuum oven overnight | | |
| | at 50° C. | | |
| 12 | Grind the crude solid into a powder using a pestle | | |
| | and mortar. | | |
| 13 | The crude solid was slurried in MeOH (5 volumes) | 3550 | mL |
| | for 2 h at reflux, allowed to cool to room | | |
| | temperature and filtered to give the title compound, | | |
| | which was dried in a vacuum oven at 50° C. to | | |
| | constant weight. | | |

Synthesis of Compounds of Formula I

### Preparative Example 1

The compounds listed below were prepared by any one of the following three general methods. The crude compounds were then purified by any one of the purification methods described below.

General Method 1

The relevant substituted 4-chloro-3-(2-chloroethyl)-2-methylquinoline (0.5 mmol; see Preparation 1 above) and the desired primary amine or aniline (1.0 mmol; see List 2 above) were heated at reflux in butanol for 48 hours. The solvent was then evaporated prior to purification of the residue.

General Method 2

The relevant substituted 4-chloro-3-(2-chloroethyl)-2-methylquinoline (0.2 mmol; see Preparation 1 above) and the desired primary amine or aniline (0.4 mmol; see List 2 above) were dissolved in ethanol or n-butanol and heated to 170° C. in a sealed tube for up to 48 hours. The solvent was then evaporated prior to purification of the residue.

General Method 3

The relevant substituted 4-chloro-3-(2-chloroethyl)-2-methylquinoline (0.55 mmol; see Preparation 1 above), and the desired primary amine or aniline (0.55 mmol; see List 2 above) were dissolved in n-butanol or ethoxyethanol and heated to 220° C., using microwave irradiation, for 20 min. The solvent was then evaporated prior to purification of the residue.

Purification Method 1

The crude substituted 4-methyl-2,3-dihydro-1H-pyrrolo[3,2-c]quinoline (obtained by any one of the three general methods described above) was purified by preparative HPLC using a 150×20.6 mm 7 micron Genesis C18 column eluting at 10 mL/min with a gradient of water/MeCN (+0.1% trifluoroacetic acid or 0.1% formic acid). The fractions containing the desired product were concentrated in vacuo to give the desired product as a trifluoroacetate or formate salt.

Purification Method 2

The crude substituted 4-methyl-2,3-dihydro -1H-pyrrolo[3,2-c]quinoline (obtained by any one of the three general methods described above) was purified by automated preparative HPLC using a 250×10 mm 10 micron Luna C18 column eluting at 8 mL/min with a gradient of MeCN/water (+0.1% formic acid). The fractions containing the desired product were concentrated in vacuo to give the desired product as a formic acid salt.

Purification Method 3

The crude substituted 4-methyl-2,3-dihydro-1H-pyrrolo[3,2-c]quinoline (obtained by any one of the three general methods described above) was purified by flash chromatography eluting with dichloromethane/methanol/acetic acid/water (240:70:3:2). The fractions containing the desired product were concentrated in vacuo to give the desired product as the free base.

Purification Method 4

The crude substituted 4-methyl-2,3-dihydro-1H-pyrrolo[3,2-c]quinoline (obtained by any one of the three general methods described above) was purified by flash chromatography eluting with a mixture of methanol and dichloromethane (from 1:99 up to 1:4). The fractions containing the desired product were concentrated in vacuo to give the desired product as the free base.

### (a) 6,8-Dimethoxy-4-methyl-1-(3-phenoxyphenyl)-2,3-dihydro-1H-pyrrolo[3,2-c]quinoline trifluoroacetate

Prepared using General Method 1 and Purification Method 1.

LCMS (Method B): Rt=8.62 min, m/z=413.12 [M+H]⁺; C₂₆H₂₄N₂O₃, Mono-isotopic mass=412.18.

¹H-NMR (400 MHz, D₆-DMSO): δ 12.95 (s, 1H), 7.60 (t, J=8.1 Hz, 1H), 7.41 (m, 2H), 7.31 (ddd, J=8.1, 2.2, 0.9 Hz, 1H), 7.24 (t, J=2.2 Hz, 1H), 7.18 (m, 1H), 7.13 (ddd, J=8.1, 2.2, 0.9 Hz, 1H), 7.09 (d, J=2.4 Hz, 1H), 7.06 (m, 2H), 5.97 (d, J=2.4 Hz, 1H), 4.39 (t, J=9.4 Hz, 2H), 4.06 (s, 3H), 3.46 (s, 3H), 3.31 (t, J=9.4 Hz, 2H), 2.61 (s, 3H).

### (b) 6,8-Dimethoxy-4-methyl-1-(2-phenoxyethyl)-2,3-dihydro-1H-pyrrolo[3,2-c]quinoline trifluoroacetate

Prepared using General Method 2 and Purification Method 1.

LCMS (Method B): Rt=7.77 min, m/z=365.12 [M+H]⁺; C₂₂H₂₄N₂O₃, Mono-isotopic mass=364.18.

¹H-NMR (400 MHz, D₆-DMSO): δ 12.5 (s, 1H), 7.32 (d, J=2.3 Hz, 1H), 7.27 (m, 2H), 7.15 (d, J=2.3 Hz, 1H), 6.94 (m, 1H), 6.90 (m, 2H), 4.42 (t, J=5.5 Hz, 2H), 4.35 (t, J=5.5 Hz, 2H), 4.19 (t, J=9.7 Hz, 2H), 4.07 (s, 3H), 3.87 (s, 3H), 3.15 (t, J=9.7 Hz, 2H), 2.51 (s, 3H).

### (c) 1-Cyclopropyl-6,8-dimethoxy-4-methyl-2,3-dihydro-1H-pyrrolo[3,2-c]quinoline trifluoroacetate

Prepared using General Method 2 and Purification Method 1.

LCMS (Method B): Rt=6.42 min, m/z=285.12 [M+H]⁺; C₁₇H₂₀N₂O₂, Mono-isotopic mass=284.15.

¹H-NMR (400 MHz, D₆-DMSO): δ 12.54 (s, 1H), 7.83 (d, J=2.4 Hz, 1H), 7.15 (d, J=2.4 Hz, 1H), 4.06 (s, 3H), 4.03 (t, J=9.4 Hz, 2H), 3.91 (s, 3H), 3.43 (m, 1H), 3.06 (t, J=9.4 Hz, 2H), 2.50 (s, 3H), 1.12 (m, 2H), 1.06 (m, 2H).

### (d) 8-Methoxy-4-methyl-1-(4-phenoxyphenyl)-2,3-dihydro-1H-pyrrolo[3,2-c]quinoline trifluoroacetate

Prepared using General Method 3 and Purification Method 1.

LCMS (Method B): Rt=8.48 min, m/z=383.11 [M+H]⁺; C₂₅H₂₂N₂O₂, Mono-isotopic mass=382.17.

¹H-NMR (400 MHz, D₄-methanol): δ 7.70 (d, J=9.5 Hz, 1H), 7.53 (m, 2H), 7.43 (m, 3H), 7.21 (m, 1H), 7.2 (m, 2H), 7.07 (m, 2H), 6.50 (d, J=2.8 Hz, 1H), 4.42 (t, J=9.5 Hz, 2H), 3.48 (s, 3H), 3.40 (t, J=9.5 Hz, 2H), 2.60 (s, 3H).

### (e) {2-[4-(8-Methoxy-4-methyl-2,3-dihydro-1H-pyrrolo[3,2-c]quinolin-1-yl)phenyoxy]ethyl}dimethylamine hydrochloride

Prepared using General Method 2 and Purification Method 1. The product was then converted to the hydrochloride salt by the addition of 1 N hydrochloric acid followed by evaporation.

LCMS (Method B): Rt=4.63 min, m/z=378.18 [M+H]⁺; C₂₃H₂₇N₃O₂, Mono-isotopic mass=377.21.

¹H-NMR (400 MHz, D₄-methanol): δ 7.72 (d, J=9.3 Hz, 1H), 7.55 (m, 2H), 7.42 (dd, J=9.3, 2.7 Hz, 1H), 7.29 (m, 2H), 6.44 (d, J=2.7 Hz, 1H), 4.47 (t, J=4.9 Hz, 2H), 4.40 (t, J=9.4 Hz, 2H), 3.67 (t, J=4.9 Hz, 2H), 3.41 (s, 3H), 3.40 (t, J=9.4 Hz, 2H), 3.02 (s, 6H), 2.61 (s, 3H).

### (f) 8-Methoxy-4-methyl-1-[4-(pyridin-3-yloxy)phenyl]-2,3-dihydro-1H-pyrrolo[3,2-c]quinoline trifluoroacetate

Prepared using General Method 3 and Purification Method 1.

LCMS (Method B): Rt=6.60 min, m/z=384.12 [M+H]⁺; C₂₄H₂₁N₃O₂, Mono-isotopic mass=383.16

¹H-NMR (400 MHz, D₄-methanol): δ 8.54 (d, J=2.9 Hz, 1H), 8.51 (dd, J=5, 1.3 Hz, 1H), 7.86 (ddd, J=8.6, 2.9, 1.3 Hz, 1H), 7.75 (d, J=9.4 Hz, 1H), 7.75 (ddd, J=8.6, 5.0, 0.6 Hz, 1H), 7.65 (m, 2H), 7.45 (dd, J=9.4, 2.8 Hz, 1H), 7.38 (m, 2H), 6.50 (d, J=2.8 Hz, 1H), 4.45 (t, J=9.5 Hz, 2H), 3.50 (s, 3H), 3.43 (t, J=9.5 Hz, 2H), 2.63 (s, 3H).

### (g) 4-Methyl-8-phenoxy-1-phenyl-2,3-dihydro-1H-pyrrolo[3,2-c]quinoline hydrochloride

Prepared using General Method 2 and Purification Method 3.

LCMS (Method B): Rt=7.95 min, m/z=353.10 [M+H]⁺; C₂₄H₂₀N₂O, Mono-isotopic mass=352.16.

¹H-NMR (400 MHz, D₆-DMSO): δ 14.26 (s, 1H), 8.03 (d, J=9.2 Hz, 1H), 7.60 (dd, J=9.2, 2.6 Hz, 1H), 7.32 (m, 7H), 7.18 (m, 1H), 6.85 (m, 2H), 6.29 (d, J=2.6 Hz, 1H), 4.28 (t, J=9.5 Hz, 2H), 3.30 (t, J=9.5 Hz, 2H), 2.60 (s, 3H).

### (h) 1-Benzyl-4-methyl-8-phenoxy-2,3-dihydro-1H-pyrrolo[3,2-c]quinoline

Prepared using General Method 2 and Purification Method 3.

LCMS (Method B): Rt=8.17 min, m/z=367.13 [M+H]⁺; C₂₅H₂₂N₂O, Mono-isotopic mass=366.17.

¹H-NMR (400 MHz, D₆-DMSO): δ 7.97 (d, J=9.3 Hz, 1H), 7.58 (dd, J=9.3, 2.4 Hz, 1H), 7.33 (m, 3H), 7.27 (m, 3H), 7.17 (m, 1H), 7.07 (m, 2H), 6.90 (m, 2H), 4.97 (s, 2H), 4.12 (t, J=9.6 Hz, 2H), 3.23 (t, J=9.6 Hz, 2H), 2.53 (s, 3H).

### (i) 1-(Indan-2-yl)-4-methyl-8-phenoxy-2,3-dihydro-1H-pyrrolo[3,2-c]quinoline

Prepared using General Method 2 and Purification Method 3.

LCMS (Method B): Rt=8.87 min, m/z=393.17 [M+H]⁺; C₂₇H₂₄N₂O, Mono-isotopic mass=392.19.

¹H-NMR (400 MHz, D₆-DMSO): δ 7.95 (d, J=9.3 Hz, 1H), 7.75 (d, J=2.5 Hz, 1H), 7.56 (dd, J=9.3, 2.5 Hz, 1H), 7.39 (m, 2H), 7.24 (m, 2H), 7.19 (m, 2H), 7.14(m, 1H), 7.10 (m, 2H), 5.24 (m, 1H), 3.78 (t, J=9.5 Hz, 2H), 3.18 (dd, J=16.2, 5.7 Hz, 2H), 3.11 (dd, J=16.2, 7.5 Hz, 2H), 3.02 (t, J=9.5 Hz, 2H), 2.45 (s, 3H).

### (j) 4-Methyl-6-phenoxy-1-phenyl-2,3-dihydro-1H-pyrrolo[3,2-c]quinoline hydrochloride

Prepared using General Method 2 and Purification Method 3.

LCMS (Method B): Rt=8.11 min, m/z=353.12 [M+H]⁺; C₂₄H₂₀N₂O, Mono-isotopic mass=352.16.

¹H-NMR (400 MHz, D₆-DMSO): δ 13.15 (s, 1H), 7.60 (m, 5H), 7.51 (m, 2H), 7.30 (m, 1H), 7.21 (m, 2H), 7.16 (dd, J=8.5, 7.9 Hz, 1H), 7.08 (dd, J=7.9, 1.3 Hz, 1H), 6.68 (dd, J=8.5, 1.3 Hz, 1H), 4.44 (t, J=9.5 2H), 3.39 (t, J=9.5 2H), 2.68 (s, 3H).

### (k) 1-Benzyl-4-methyl-6-phenoxy-2,3-dihydro-1H-pyrrolo[3,2-c]quinoline hydrochloride

Prepared using General Method 2 and Purification Method 3.

LCMS (Method B): Rt=8.28 min, m/z=367.16 [M+H]⁺; C₂₅H₂₂N₂O, Mono-isotopic mass=366.17

¹H-NMR (400 MHz, D₆-DMSO): δ 12.84 (s, 1H), 7.75 (dd, J=8.8, 0.9 Hz, 1H), 7.49 (m, 2H), 7.39 (m, 4H), 7.30 (m, 3H), 7.19 (m, 2H), 7.10 (dd, J=7.9, 0.9 Hz, 1H), 5.24 (s, 2H), 4.18 (t, J=9.6 Hz, 2H), 3.27 (t, J=9.6 Hz, 2H), 2.58 (s, 3H).

### (1) 1-(Indan-2-yl)-4-methyl-6-phenoxy-2,3-dihydro-1H-pyrrolo[3,2-c]quinoline hydrochloride

Prepared using General Method 2 and Purification Method 3.

LCMS (Method B): Rt=8.95 min, m/z=393.17 [M+H]⁺; C₂₇H₂₄N₂O, Mono-isotopic mass=392.19.

¹H-NMR (400 MHz, D₆-DMSO): δ 12.75 (s, 1H), 8.20 (d, J=8.9 Hz, 1H), 7.49 (m, 3H), 7.31 (m, 3H), 7.22 (m, 5H), 5.61 (m, 1H), 3.94 (t, J=9.4 Hz, 2H), 3.42 (dd, J=16.3, 7.5 Hz, 2H), 3.32 (dd, J=16.3, 5.5 Hz, 2H), 3.10 (t, J=9.4 Hz, 2H), 2.53 (s, 3H).

### (m) 4-Methyl-1-(2-phenylethyl)-8-phenoxy-2,3-dihydro-1H-pyrrolo[3,2-c]quinoline hydrochloride

Prepared using General Method 2 and Purification Method 3. The product was then converted to the hydrochloride salt by addition of 1 N hydrochloric acid, followed by evaporation.

LCMS (Method B): Rt=8.58 min, m/z=381.11 [M+H]⁺; C₂₆H₂₄N₂O, Mono-isotopic mass=380.19.

¹H-NMR (400 MHz, D₆-DMSO): δ 13.91 (s, 1H) 8.03 (d, J=9.3 Hz, 1H), 7.67 J=9.3, 2.4 Hz, 1H), 7.56 (d, J=2.4 Hz, 1H), 7.45 (m, 2H), 7.21 (m, 4H), 7.14 (m, 2H), 7.02 (m, 2H), 3.96 (t, J=9.6 Hz, 2H), 3.89 (t, J=7.6 Hz, 2H), 3.08 (t, J=9.6 Hz, 2H), 2.89 (t, J=7.6 Hz, 2H), 2.49 (s, 3H)

### (n) 8-Methoxy-4-methyl-1-(4-phenoxyphenyl)-2,3-dihydro-1H-pyrrolo[3,2-c]quinoline hydrochloride A

Prepared using General Method 2 and Purification Method 1. The product was then converted to the hydrochloride salt by the addition of 1 N hydrochloric acid, followed by evaporation.

LCMS Method B; Rt=8.48 min, M+=383.15 [M+H] +; C₂₅H₂₂N₂O₂, Mono-isotopic mass=382.17.

¹H-NMR (400 MHz, D₆-DMSO): δ 14.22 (s, 1H), 7.97 (d, J=9.3 Hz, 1H), 7.60 (m, 2H), 7.51 (dd, J=9.3, 2.7 Hz, 1H), 7.46 (m, 2H), 7.22 (m, 3H), 7.07 (m, 2H), 6.36 (d, J=2.7 Hz, 1H), 4.38 (t, J=9.5 Hz, 2H), 3.45 (s, 3H), 3.34 (t, J=9.5 Hz, J=2 H), 2.61 (s, 3H).

### (o) 8-Methoxy-4-methyl-1-(2-phenylethyl)-2,3-dihydro-1H-pyrrolo[3,2-c]quinolin-6-ol trifluoroacetate

Prepared using General Method 2 and Purification Method 1 (a side-product from the preparation of Preparative Example 3(viii) below).

LCMS (Method B): Rt=7.42 min, m/z=335.12 [M+H]⁺; C₂₁H²²N₂O₂, Mono-isotopic mass=334.17.

¹H-NMR (400 MHz, D₆-DMSO): δ 12.45 (s, 1H), 11.79 (s, 1H), 7.32 (m, 4H), 7.23 (m, 1H), 6.89 (d, J=2.4 Hz, 1H), 6.86 (d, J=2.4 Hz, 1H), 4.14 (t, J=7.5 Hz, 2H), 4.00 (t, J=9.7 Hz, 2H), 3.81 (s, 3H), 3.11 (t, J=7.5 Hz, 2H), 3.08 (t, J=9.7 Hz, 2.49 (s, 3H).

### (p) 1-(1-Benzyl-piperidin-4-yl)-4-methyl-8-phenoxy-2,3-dihydro-1H-pyrrolo[3,2-c]quinoline bis-trifluoroacetate

Prepared using General Method 2 and Purification Method 1.

LCMS (Method B): Rt=5.90 min, m/z=450.22 [M+H]⁺; C₃₀H₃₁N₃O, Mono-isotopic mass=449.25

¹H-NMR (400 MHz, D₆-DMSO, NaOD): δ 7.79 (d, J=9.2 Hz, 1H), 7.46 (m, 2H), 7.35 (m, 3H), 7.27 (m, 5H), 7.14 (m, 2H), 3.71 (m, 1H), 3.64 (t, J=9.4 Hz, 2H), 3.40 (s, 2H), 2.97 (t, J=9.4 Hz, 2H), 2.74 (d, br, J=10.6 Hz, 2H), 2.36 (s, 3H), 1.63 (m, 6H).

### (q) 1-(Indan-1-yl)-4-methyl-8-phenoxy-2,3-dihydro-1H-pyrrolo[3,2-c]quinoline hydrochloride

Prepared using General Method 2 and Purification Method 3.

LCMS (Method B): Rt=9.05 min, m/z=393.09 [M+H]⁺; C₂₇H₂₄N2O, Mono-isotopic mass=392.19.

¹H-NMR (400 MHz, D₆-DMSO): δ 13.92 (s, 1H), 8.04 (d, J=9.3 Hz, 1H), 7.77 (s, 1H), 7.67 (dd, J=9.3, 1.8 Hz, 1H), 7.41 (m, 2H), 7.32 (m, 2H), 7.24 (m, 2H), 7.18 (m, 1H), 7.11 (m, 2H), 6.00 (t, J=7.0 Hz, 1H), 3.72 (m, 1H), 3.57 (m, 1H), 3.05 (m, 3H), 2.83 (m, 1H), 2.50 (s, 3H), 2.22 (m, 2H)

### (r) 1-(Benzodioxan-2-ylmethyl)-4-methyl-8-phenoxy-2,3-dihydro-1H-pyrrolo[3,2-c]quinoline hydrochloride

Prepared using General Method 2 and Purification Method 3.

LCMS (Method B): Rt=8.57 min, m/z=425.10[M+H]⁺; C₂₇H₂₄N₂O₃, Mono-isotopic mass=424.18.

¹H-NMR (400 MHz, D₆-DMSO): δ 13.99 (s, 1H), 8.02 (d, J=9.3 Hz, 1H), 7.72 (d, J=2.4 Hz, 1H), 7.67 (dd, J=9.3, 2.4 Hz, 1H), 7.27 (m, 2H), 7.11 (m, 1H), 7.05 (m, 2H), 6.88 (dd, J=8.0, 1.7 Hz, 1H), 6.83 (ddd, J=8.0, 7.1, 1.7 Hz, 1H), 6.77 (ddd, J=8.0, 7.1, 1.7 Hz, 1H), 6.57 (dd, J=8.0, 1.7 Hz, 1H), 4.57 (m, 1H), 4.25 (dd, J=11.5, 2.3 Hz, 1H), 4.18 (m, 1H), 4.02 (m, 2H), 3.92 (dd, J=15.8, 3.7 Hz, 1H), 3.72 (dd, J=11.5, 7.3 Hz, 1H), 3.17 (m, 2H), 2.52 (s, 3H).

### (s) 4-Methyl-8-phenoxy-1-(1,2,3,4-tetrahydronaphthalen-1-yl)-2,3-dihydro-1H-pyrrolo[3,2-c]quinoline hydrochloride

Prepared using General Method 2 and Purification Method 3.

LCMS (Method B): Rt=9.12 min, m/z=407.20 [M+H]⁺; C₂₈H₂₆N₂O, Mono-isotopic mass=406.20

¹H-NMR (400 MHz, D₆-DMSO): δ 13.90 (s, br, 1H), 8.02 (d, J=9.3 Hz, 1H), 7.67 (d, br, J=9.3 Hz, 1H), 7.48 (s, br, 1H), 7.36 (m, 2H), 7.13 (m, 7H), 5.53 (s, br, 1H), 3.90 (s, br, 1H), 3.56 (s, br, 1H), 3.10 (t, J=9.6 Hz, 2H), 2.73 (m, br, 2H), 2.52 (s, 3H), 1.99 (m, br, 2H), 1.90 (m, 1H), 1.68 (m, 1H).

### (t) 1-Cyclohexyl-4-methyl-8-phenoxy-2,3-dihydro-1H-pyrrolo[3,2-c]quinoline

Prepared using General Method 3 and Purification Method 4.

LCMS (Method B): Rt=8.83 min, m/z=359.17 [M+H]⁺; C₂₄H₂₆N₂O, Mono-isotopic mass=358.20.

¹H-NMR (400 MHz, D₆-DMSO): δ 13.80 (s, 1H), 8.02 (d, J=9.4 Hz, 1H), 7.72 (dd, J=9.4, 2.6 Hz, 1H), 7.50 (m, 2H), 7.39 (d, J=2.6 Hz, 1H), 7.27 (m, 1H), 7.21 (m, 2H), 3.98 (t, J=9.6 Hz, 2H), 3.96 (m, 1H), 3.07 (t, J=9.6 Hz, 2H), 2.47 (s, 1.75 (d, J=12.0 Hz, 2H), 1.57 (m, 5H), 1.07 (m, 1H), 0.87 (m, 2H).

### (u) 8-Ethoxy-4-methyl-1-(4-phenoxyphenyl)-2,3-dihydro-1H-pyrrolo[3,2-c]quinoline formate

Prepared using General Method 2 and Purification Method 2.

LCMS (Method B): Rt=8.94 min, m/z=397.15 [M+H]⁺; C₂₆H₂₄N₂O₂, Mono-isotopic mass=396.18.

¹H-NMR (400 MHz, D₄-methanol): δ 8.50 (br s, 1H), 7.71 (d, J=9.3 Hz, 1H), 7.57 (t, J=8.1 Hz, 1H), 7.42 (dd, J=9.3, 2.6 Hz, 1H), 7.37 (m, 2H), 7.24 (ddd, J=8.1, 2.2, 0.9 Hz, 1H), 7.16 (m, 1H), 7.12 (ddd, J=8.1, 2.2, 0.9 Hz, 1H), 7.09 (t, J=2.2 Hz, 1H), 7.04 (m, 2H), 6.50 (d, J=2.6 Hz, 1H), 4.39 (t, J=9.5 Hz, 2H), 3.67 (q, J=7.0 Hz, 2H), 3.37 (t, J=9.5 Hz, 2H), 2.59 (s, 3H), 1.28 (t J=7.0 Hz, 3H).

### Preparative Example 2

The following compounds were prepared, from appropriate intermediates (such as those described hereinbefore), according to or by analogy with methods described herein:

### (i) 1-(4-methoxyphenyl)-4-methyl-8-phenoxy-2,3-dihydro-1H-pyrrolo[3,2-c]quinoline trifluoroacetate

Prepared using General Method 3 and Purification Method 1.

LCMS (Method B): Rt=8.14 min, m/z=383.11 [M+H]⁺; C₂₅H₂₂N₂O₂, Mono-isotopic mass=382.17.

¹H-NMR (400 MHz, D₄-methanol): δ 7.77 (d, J=9.2 Hz, 1H), 7.59 (dd, J=9.2, 2.6 Hz, 1H), 7.30 (m, 2H), 7.18 (m, 3H), 6.82 (m, 4H), 6.41 (d, J=2.6 Hz, 1H), (t, J=9.5 Hz, 2H), 3.84 (s, 3H), 3.33 (t, J=9.5 Hz, 2H), 2.58 (s, 3H).

### (ii) 4-methyl-1-(4-phenoxyphenyl)-2,3-dihydro-1H-pyrrolo[3,2-c]quinoline hydrochloride

Prepared using General Method 2 and Purification Method 1. The product was then converted to the hydrochloride salt by the addition of 1 N hydrochloric acid, followed by evaporation.

LCMS (Method B): Rt=8.34 min, m/z=353.10 [M+H]⁺; C₂₄H₂₀N₂O, Mono-isotopic mass=352.16. ¹H-NMR (400 MHz, D₆-DMSO): δ 14.0 (s, 1H), 7.99 (d, J=8.7 Hz, 1H), 7.82 (m, 1H), 7.58 (m, 2H), 7.47 (m, 2H), 7.34 (m, 1H), 7.23 (m, 1H), 7.20 (m, 2H), 7.16 (m, 2H), 7.06 (dd, J=8.7, 1.2 Hz, 1H), 4.38 (t, J=9.4 Hz, 2H), 3.34 (t, J=9.4 Hz, 2.62 (s, 3H).

### (iii) 4-methyl-1-(2-methylphenyl)methyl-8-phenoxy-2,3-dihydro-1H-pyrrolo[3,2-c]quinoline hydrochloride

Prepared using General Method 2 and Purification Method 1. The product was then converted to the hydrochloride salt by the addition of 1 N hydrochloric acid, followed by evaporation.

LCMS (Method B): Rt=8.48 min, m/z=381.17 [M+H]⁺; C₂₆H₂₄N₂O, Mono-isotopic mass=380.19.

¹H-NMR (400 MHz, D₆-DMSO): δ 13.94 (s, br, 1H), 8.01 (d, J=9.2 Hz, 1H), 7.63 (dd, J=9.2, 2.4 Hz, 1H), 7.25 (m, 2H), 7.16 (m, 3H), 7.10 (m, 1H), 7.00 (m, 2H), 6.80 (m, 2H), 4.88 (s, 2H), 4.11 (t, J=9.6 Hz, 2H), 3.26 (t, J=9.6 Hz, 2H), 2.55 (s, 3H), 2.02 (s, 3H).

### (iv) 4-methyl-8-phenoxy-1-(4-iso-propylphenyl)-2,3-dihydro-1H-pyrrolo[3,2-c]quinoline hydrochloride

Prepared using General Method 2 and Purification Method 3. The product was then converted to the hydrochloride salt by the addition of 1 N hydrochloric acid, followed by evaporation.

LCMS (Method B): Rt=9.29 min, m/z=395.18 [M+H]⁺; C₂₇H₂₆N₂O, Mono-isotopic mass=394.20.

¹H-NMR (400 MHz, D₆-DMSO): δ 14.1 (s, br, 1H), 8.05 (d, J=9.2 Hz, 1H), 7.64 (dd, J=9.2, 2.6 Hz, 1H), 7.32 (m, 2H), 7.30 (m, 2H), 7.23 (m, 2H), 7.13 (m, 1H), 6.86 (m, 2H), 6.49 (d, J=2.6 Hz, 1H), 4.30 (t, J=9.5 Hz, 2H), 3.31 (t. J=9.5 Hz, 2H), 2.88 (hept, J=7.0 Hz, 1H), 2.61 (s, 3H), 1.16 (d, J=7.0 Hz, 6H).

### (v) 4-methyl-8-phenoxy-1-(1-phenylethyl)-2,3-dihydro-1H-pyrrolo[3,2-c]quinoline trifluoroacetate

Prepared using General Method 2 and Purification Method 1.

LCMS (Method B): Rt=8.60 min, m/z=381.17 [M+H]⁺; C₂₆H₂₄N₂O, Mono-isotopic mass=380.19.

¹H-NMR (400 MHz, D₆-DMSO): δ 13.5 (s, br, 1H), 7.86 (d, J=9.2 Hz, 1H), 7.61 (dd, J=9.2, 2.6 Hz, 1H), 7.53 (d, J=2.6 Hz, 1H), 7.41 (m, 2H), 7.29 (m, 3H), 7.23 (m, 1H), 7.16 (m, 2H), 6.97 (m, 2H), 5.69 (q, J=6.8 Hz, 1H), 4.20 (m, 1H), 3.96 (m, 1H), 3.19 (t, J=9.6 Hz, 2H), 2.50 (s, 3H), 1.64 (d, J=6.8 Hz, 3H).

### (vi) 8-methoxy-4-methyl-1-(2-phenylethyl)-2,3-dihydro-1H-pyrrolo[3,2-c]quinoline formate

Prepared using General Method 1 and Purification Method 2.

LCMS (Method B): Rt=7.44 min, m/z=319.14 [M+H]⁺; C₂₁H₂₂N₂O, Mono-isotopic mass=318.17.

¹H-NMR (400 MHz, D₄-methanol): δ 8.45 (s, 1H), 7.69 (d, J=9.2 Hz, 1H), 7.50 (dd, J=9.2, 2.6 Hz, 1H), 7.42 (d, J=2.6 Hz, 1H), 7.29 (m, 4H), 7.22 (m, 1H), 4.22 (t, J=7.2 Hz, 2H), 4.00 (t, J=9.6 Hz, 2H), 3.87 (s, 3H), 3.21 (t, J=7.2 Hz, 2H), 3.14 (t, J=9.6 Hz, 2H), 2.49 (s, 3H).

### Preparative Example 3

The following compounds were prepared, from appropriate intermediates (such as those described hereinbefore), according to or by analogy with methods described herein:

### (i) 6,8-dimethoxy-1-(4-hydroxyphenyl)-4-methyl-2,3-dihydro-1H-pyrrolo[3,2-c]quinoline

Prepared using General Method 2 and Purification Method 3.

LCMS (Method B): Rt=6.43 min, m/z=337.13 [M+H]⁺; C₂₀H₂₀N₂O₃, Mono-isotopic mass=336.15.

¹H-NMR (400 MHz, D₆-DMSO): δ 9.50 (s, br, 1H), 7.07 (m, 2H), 6.81 (m, 2H), 6.53 (d, J=2.6 Hz, 1H), 5.90 (d, J=2.6 Hz, 1H), 3.95 (t, J=9.2 Hz, 2H), 3.84 (s, 3H), 3.32 (s, 3H), 3.16 (t, J=9.2 Hz, 2H), 2.42 (s, 3H).

### (ii) 6,8-dimethoxy-1-(3-hydroxyphenyl)-4-methyl-2,3-dihydro-1H-pyrrolo[3,2-c]quinoline formate

Prepared using General Method 2 and Purification Method 2.

LCMS (Method B): Rt=6.68 min, m/z=337.09 [M+H]⁺; C₂₀H₂₀N₂O₃, Mono-isotopic mass=336.15.

¹H-NMR (400 MHz, D₆-DMSO): δ 8.18 (s, 1H), 7.21 (t, J=8.0 Hz, 1H), 6.65 (m, 3H), 6.58 (t, J=2.2 Hz, 1H), 6.04 (d, J=2.5 Hz, 1H), 4.13 (t, J=9.2 Hz, 2H), 3.90 (s, 3H), 3.39 (s, 3H), 3.21 (t, J=9.2 Hz, 2H), 2.48 (s, 3H).

### (iii) 6,8-dimethoxy-1-(3-hydroxy-5-methylphenyl)-4-methyl-2,3-dihydro-1H-pyrrolo[3,2-c]quinoline formate

Prepared using General Method 2 and Purification Method 2.

LCMS (Method B): Rt=7.14 min, m/z=351.12 [M+H]⁺; C₂₁H₂₂N₂O₃, Mono-isotopic mass=350.16.

¹H-NMR (400 MHz, D₆-DMSO): δ 8.18 (s, 1H), 6.64 (d, J=2.5 Hz, 1H), 6.46 (m, 2H), 6.36 (t, J=2.0 Hz, 1H), 6.08 (d, J=2.5 Hz, 1H), 4.11 (t, J=9.2 Hz, 2H), 3.89 (s, 3H), 3.41 (s, 3H), 3.20 (t, J=9.2 Hz, 2H), 2.46 (s, 3H), 2.21 (s, 3H).

### (iv) 8-methoxy-1-(4-methoxyphenyl)-4-methyl-2,3-dihydro-1H-pyrrolo[3,2-c]quinoline trifluoroacetate

Prepared using General Method 3 and Purification Method 1.

LCMS (Method B): Rt=7.03 min, m/z=321.12 [M+H]⁺; C₂₀H₂₀N₂O₂, Mono-isotopic mass=320.15

¹H-NMR (400 MHz, D₆-DMSO): δ 13.55 (s, 1H), 7.79 (d, J=9.3 Hz, 1H), 7.52 (m, 2H), 7.49 (dd, J=9.3, 2.8 Hz, 1H), 7.17 (m, 2H), 6.31 (d, J=2.8 Hz, 1H), 4.35 (t, J=9.5 Hz, 2H), 3.83 (s, 3H), 3.36 (s, 3H), 3.33 (t, J=9.5 Hz, 2H), 2.57 (s, 3H).

### (v) 8-trifluoromethoxy-1-(4-phenoxyphenyl)-4-methyl-2,3-dihydro-1H-pyrrolo[3,2-c]quinoline hydrochloride

Prepared using General Method 2 and Purification Method 1. The product was then converted to the hydrochloride salt by the addition of 1 N hydrochloric acid, followed by evaporation.

LCMS (Method B): Rt=8.95 min, m/z=437.10 [M+H]⁺; C₂₅H₁₉F₃N₂O₂, Mono-is mass=436.14.

¹H-NMR (400 MHz, D₆-DMSO): δ 14.4 (s, br, 1H), 8.17 (d, J=9.4 Hz, 1H), 7.86 (dd, J=9.4, 2.6 Hz, 1H), 7.62 (m, 2H), 7.46 (m, 2H), 7.23 (m, 3H), 7.09 (m, 2H), 6.80 (m, 1H), 4.41 (t, J=9.5 Hz, 2H), 3.36 (t, J=9.5 Hz, 2H), 2.64 (s, 3H).

### (vi) 6,8-dimethoxy-4-methyl-1-[4-(pyridin-3-yloxy)phenyl]-2,3-dihydro-1H-pyrrolo[3,2-c]quinoline trifluoroacetate

Prepared using General Method 2 and Purification Method 1.

LCMS (Method B): Rt=6.82 min, m/z=414.12 [M+H]⁺; C₂₅H₂₃N₃O₃, Mono-isotopic mass=413.17

¹H-NMR (400 MHz, D₆-DMSO): δ 12.9 (s, 1H), 8.45 (m, 2H), 7.61 (m, 2H), 7.52 (m, 2H), 7.30 (m, 2H), 7.07 (d, J=2.4 Hz, 1H), 5.94 (d, J=2.4 Hz, 1H), 4.39 (t, J=9.5 Hz, 2H), 4.06 (s, 3H), 3.43 (s, 3H), 3.33 (t, J=9.5 Hz, 2H), 2.62 (s, 3).

### (vii) 1-benzyl-6,8-dimethoxy-4-methyl-2,3-dihydro-1H-pyrrolo[3,2-c]quinoline formate

Prepared using General Method 2 and Purification Method 2.

LCMS (Method B): Rt=7.39 min, m/z=335.13 [M+H]⁺; C₂₁H₂₂N₂O₂, Mono-isotopic mass=334.17

¹H-NMR (400 MHz, D6-DMSO): δ 8.22 (s, 1H),7.38 (m, 4H), 7.28 (m, 1H), 6.66 (d, J=2.5 Hz, 1H), 6.62 (d, J=2.5 Hz, 1H), 4.93 (s, 2H), 3.92 (t, J=9.5 Hz, 2H), 3.87 (s, 3H), 3.44 (s, 3H), 3.14 (t, J=9.5 Hz, 2H), 2.41 (s, 3H).

### (viii) 6,8-dimethoxy-4-methyl-1-(2-phenylethyl)-2,3-dihydro-1H-pyrrolo[3,2-c]quinoline formate

Prepared using General Method 2 and Purification Method 2.

LCMS (Method B): Rt=7.82 min, m/z=349.14 [M+H]⁺; C₂₂H₂₄N₂O₂, Mono-isotopic mass=348.18.

¹H-NMR (400 MHz, D₆-DMSO): δ 8.22 (s, 1H), 7.31 (m, 4H), 7.22 (m, 1H), 6.74 (d, J=2.5 Hz, 1H), 6.72 (d, J=2.5 Hz, 1H), 3.90 (s, 3H), 3.85 (t, J=7.6 Hz, 2H), 3.77 (s, 3H), 3.76 (t, J=9.5 Hz, 2H), 3.03 (t, J=9.5 Hz, 2H), 2.97 (t, J=7.6 Hz, 2.38 (s, 3H).

### (ix) 4-methyl-1-(2-phenylethyl)-8-trifluoromethoxy-2,3-dihydro-1H-pyrrolo[3,2-c]quinoline hydrochloride

Prepared using General Method 2 and Purification Method 1. The product was then converted to the hydrochloride salt by the addition of 1 N hydrochloric acid, followed by evaporation.

LCMS (Method B): Rt=8.09 min, m/z=373.08 [M+H]⁺; C₂₁H₁₉F₃N₂O, Mono-isotopic mass=372.14

¹H-NMR (400 MHz, D6-DMSO): 13.95 (s, br, 1H), 8.06 (d, J=9.5 Hz, 1H), 7.99 (d, J=2.3 Hz, 1H), 7.91 (m, 1H), 7.29 (m, 4H), 7.20 (m, 1H), 4.16 (t, J=7.4 Hz, 2H), 4.06 (t, J=9.5 Hz, 2H), 3.12 (t, J=9.5 Hz, 2H), 3.08 (t, J=7.4 Hz, 2H), 2.50 (s, 3H).

### (x) 6,8-dimethoxy-1-(indan-1-yl)-4-methyl-2,3-dihydro-1H-pyrrolo[3,2-c]quinoline formate

Prepared using General Method 2 and Purification Method 2.

LCMS (Method B): Rt=8.19 min, m/z=361.14 [M+H]⁺; C₂₃H₂₄N₂O₂, Mono-isotopic mass=360.18.

¹H-NMR (400 MHz, D₆-DMSO): δ 8.20 (s, 1H), 7.32 (d, J=7.4 Hz, 1H), 7.26 (td, J=7.4, 1.5 Hz, 1H), 7.20 (t, J=7.4 Hz, 1H), 7.17 (d, J=7.4 Hz, 1H), 6.99 (d, J=2.4 Hz, 1H), 6.76 (d, J=2.4 Hz, 1H), 6.07 (t, J=7.4 Hz, 1H), 3.92 (s, 3H), 3.80 (s, 34H), 3.50 (q, J=10.0 Hz, 1H), 3.36 (td, J=10.0, 7.4 Hz, 1H), 2.97 (m, 4H), 2.45 (m, 1H), 2.40 (s, 3H), 2.13 (m, 1H).

### (xi) 6,8-dimethoxy-4-methyl-1-[(6-phenoxy)pyridin-3-yl]-2,3-dihydro-1H-pyrrolo[3,2-c]quinoline trifluoroacetate

Prepared using General Method 2 and Purification Method 1.

LCMS (Method B): Rt=7.86 min, m/z=414.12 [M+H]⁺; C₂₅H₂₃N₃O₃, Mono-isotopic mass=413.17.

¹H-NMR (400 MHz, D₆-DMSO): δ 13.01 (s, 1H), 8.38 (d, J=2.8 Hz, 1H), 8.10 (d, J=8.7, 2.8 Hz, 1H), 7.47 (m, 2H), 7.28 (d, J=8.7 Hz, 1H), 7.26 (m, 1H), 7.15 (m, 2H), 7.09 (d, J=2.4 Hz, 1H), 5.89 (d, J=2.4 Hz, 1H), 4.38 (t, J=9.5 Hz, 2H), 4,06 (s, 3H), 3.47 (s, 3H), 3.34 (t, 2H), 2.64 (s, 3H).

### (xii) 6,8-dimethoxy-1-[(6-methoxy)pyridin-3-yl]-4-methyl-2,3-dihydro-1H-pyrrolo[3,2-c]quinoline trifluoroacetate

Prepared using General Method 2 and Purification Method 1.

LCMS (Method B): Rt=6.57 min, m/z=352.13 [M+H]⁺; C₂₀H₂₁N₃O₃, Mono-isotopic mass=351.16.

¹H-NMR (400 MHz, D₆-DMSO): δ 12.96 (s, 1H), 8.43 (dd, J=2.8, 0.5 Hz, 1H), 7.95 (dd, J=8.8, 2.8 Hz, 1H), 7.08 (d, J=2.4 Hz, 1H), 7.07 (dd, J=8.8, 0.5 Hz, 1H), 5.88 (d, J=2.4 Hz, 1H), 4.36 (t, J=9.4 Hz, 2H), 4.06 (s, 3H), 3.92 (s, 3H), 3.39 (s, 3H), 3.33 (t, J=9.4 Hz, 2H), 2.63 (s, 3H).

### (xiii) 1-(benzodioxol-5-ylmethyl)-6,8-dimethoxy-4-methyl-2,3-dihydro-1H-pyrrolo[3,2-c]quinoline formate

Prepared using General Method 2 and Purification Method 2.

LCMS (Method B): Rt=7.39 min, m/z=379.16 [M+H]⁺; C₂₂H₂₂N₂O₄, Mono-isotopic mass=378.16

¹H-NMR (400 MHz, D₆-DMSO): δ 8.21 (s, 1H), 6.94 (d, J=1.7 Hz, 1H), 6.89 (d, J=7.9 Hz, 1H), 6.86 (dd, J=7.9, 1.7 Hz, 1H), 6.67 (m, 2H), 5.99 (s, 2H), 4.81 (s, 2H), 3.88 (s, 3H), 3.87 (t, J=9.5 Hz, 2H), 3.55 (s, 3H), 3.11 (t, J=9.5 Hz, 2H), 2.40 (s, 3H).

### Preparative Example 4

The following compounds were prepared, from appropriate intermediates (such as those described hereinbefore), according to or by analogy with methods described herein:

### (a) 6,8-dimethoxy-4-methyl-1-(3-methylbutyl)-2,3-dihydro-1H-pyrrolo[3,2-c]quinoline formate

Prepared using General Method 2 and Purification Method 2.

LCMS (method B): Rt=7.90 min, m/z=315.14[M+H]⁺; C₁₉H₂₆N₂O₂, Mono-isotopic mass=314.20

¹H NMR (400 MHz, D₆-DMSO) δ 8.21 (s, 1H), 6.83 (d, J=2.5 Hz, 1H), 6.79 (d, J=2.5 Hz, 1H), 3.92 (s, 3H), 3.85 (s, 3H), 3.82 (t, J=9.4 Hz, 2H), 3.66 (m, 2H), 3.04 (t, J=9.4 Hz, 2H), 2.39 (s, 3H), 1.69 (m, 1H), 1.59 (m, 2H), 0.95, (d, J=6.5 Hz, 6H).

### (b) 1-cyclopropylmethyl-6,8-dimethoxy-4-methyl-2,3-dihydro-1H-pyrrolo[3,2-c]quinoline trifluoroacetate

Prepared using General Method 2 and Purification Method 1.

LCMS (method B): Rt=7.04 min, m/z=299.13[M+H]⁺; C₁₈H₂₂N₂O₂, Mono-isotopic mass=298.17

¹H NMR (400 MHz, D₆-DMSO) δ 12.40 (br s, 1H), 7.15 (d, J=2.3 Hz, 1H), 7.13 (d, J=2.3 Hz, 1H), 4.15 (t, J=9.5 Hz, 2H), 4.07 (s, 3H), 3.92 (s, 3H), 3.86 (d, J=6.6 Hz, 2H), 3.13 (t, J=9.5/hz, 2H), 2.50 (s, 3H), 1.24 (m, 1H), 0.61 (m, 2H) 0.41 (m, 2H).

### (c) 4-methyl-8-(morpholin-4-yl)-1-(4-phenoxyphenyl)-2,3-dihydro-1H-pyrrolo[3,2-c]quinoline formate

Prepared using General Method 2 and Purification Method 2.

LCMS (method B): Rt=8.21 min, m/z=438.15[M+H]⁺; C₂₈H₂₇N₃O₂, Mono-isotopic mass=437.21

¹H NMR (400 MHz, D₄-methanol) δ 8.54 (s, 1H), 7.69 (d, J=9.5 Hz, 1H), 7.58 (dd, J=9.5, 2.6 Hz, 1H), 7.53 (t, J=8.0 Hz, 1H), 7.36 (m, 2H), 7.16 (m, 2H), 7.03 (m, 4H), 6.43 (d, J=2.6 Hz, 1H), 4.34 (t, J=9.3 Hz, 2H), 3.74 (m, 4H), 3.33 (t, J=9.3 Hz, 2H), 2.89 (m, 4H) 2.56 (s, 3H).

### (d) 8-methoxy-4-methyl-1-(1,2,3,4-tetrahydronaphthalen-1-yl)-2,3-dihydro-1H-pyrrolo[3,2-c]quinoline formate

Prepared using General Method 2 and Purification Method 2.

LCMS (method B): Rt=8.00 min, m/z=345.18[M+H]⁺; C₂₃H₂₄N₂O, Mono-isotopic mass=344.19

¹H NMR (400 MHz, D₄-methanol) δ 8.54 (s, 1H), 7.74 (d, J=9.5 Hz, 1H), 7.34 (d, J=7.0 Hz, 1H), 7.17 (m, 5H), 5.52 (br s, 1H), 3.67 (br m, 5H), 3.05 (t, J=9.4 Hz, 2H), 2.88 (m, 2H), 2.45 (s, 3H), 2.11 (m, 3H), 1.88 (m, 1H).

### (e) 4-methyl-1-(2-phenylethyl)-2,3-dihydro-1H-pyrrolo[3,2-c]quinoline hydrochloride

Prepared using General Method 2 and Purification Method 1. The product was then converted to the hydrochloride salt by the addition of 1 N hydrochloric acid, followed by evaporation.

LCMS (method B): Rt=7.25 min, m/z=289.17[M+H]⁺; C₂₀H₂₀N₂, Mono-isotopic mass=288.16

¹H NMR (400 MHz, D₆-DMSO) δ 13.59 (s, 1H), 8.2 (d, J=8.6 Hz, 1H), 7.92 (dd, J=8.7, 1.4 Hz, 1H), 7.86 (m, 1H), 7.55 (m, 1H), 7.33 (m, 4H), 7.23 (m, 1H), 4.17 (t, J=7.4 Hz, 2H), 3.99 (m, 2H), 3.09 (m, 4H), 2.49 (s, 3H).

### (f) 4,6-dimethyl-1-(2-methylphenyl)-2,3-dihydro-1H-pyrrolo[3,2-c]quinoline hydrochloride

Prepared using General Method 2 and Purification Method 1. The product was then converted to the hydrochloride salt by the addition of 1 N hydrochloric acid, followed by evaporation.

LCMS (method B): Rt=7.25 min, m/z=289.15[M+H]⁺; C₂₀H₂₀N₂, Mono-isotopic mass=288.16

¹H NMR (400 MHz, D₆-DMSO) δ 12.06 (s, 1H), 7.66 (dt, j=7.0, 1.1 Hz, 1H), 7.52 (m, 3H), 7.46 (m, 1H), 7.14 (dd, J=8.6, 7.0 Hz, 1H), 6.68 (d, J=8.6 Hz, 1H), 4.33 (m, 2H), 3.40 (m, 2H), 2.71 (s, 3H), 2.67 (s, 3H), 2.18 (s, 3H).

### (g) 4,6-dimethyl-1-(2-phenylethyl)-2,3-dihydro-1H-pyrrolo[3,2-c]quinoline hydrochloride

Prepared using General Method 2 and Purification Method 1. The product was then converted to the hydrochloride salt by the addition of 1 N hydrochloric acid, followed by evaporation.

LCMS (method B): Rt=7.60 min, m/z=303.19[M+H]⁺; C₂₁ H₂₂N₂, Mono-isotopic mass=302.18

¹H NMR (400 MHz, D₆-DMSO) δ 11.73 (s, 1H), 8.08 (d, J=8.6 Hz, 1H), 7.74 (dt, J=7.2, 1.0 Hz, 1H), 7.47 (dd, J=8.6, 7.2 Hz, 1H), 7.33 (m, 4H), 7.24 (m, 1H), 4.16 (t, J=7.5 Hz, 2H), 4.00 (m, 2H), 3.09 (m, 4H), 2.67 (s, 3H), 2.58 (s, 3H).

### (h) 4-methyl-8-(piperidin-1-yl)-1-[4-(piperidin-1-yl)phenyl]-2,3-dihydro-pyrrolo[3,2-c]quinoline hydrochloride

Prepared using General Method 2 and Purification Method 1. The product was then converted to the hydrochloride salt by the addition of 1 N hydrochloric acid, followed by evaporation.

LCMS (method B): Rt=8.10 min, m/z=427.17[M+H]⁺; C₂₈H₃₄N₄, Mono-isotopic mass=426.28 ¹H NMR (400 MHz, D₄-methanol) 6 7.92 (m, 4H), 7.73 (m, 2H), 7.15 (br s, 1H), 4.48 (t, J=9.4 Hz, 2H), 3.75 (t, J=5.5 Hz, 4H), 3.44 (t, J=9.4 Hz, 2H), 3.22 (br t, J=5.5 Hz, 4H), 2.66 (s, 3H), 2.12 (m, 4H), 1.83 (m, 6H), 1.66 (m, 2H).

### (i) 4-methyl-8-(piperidin-1-yl)-1-(3-phenoxyphenyl)-2,3-dihydro-1H-pyrrolo[3,2-c]quinoline hydrochloride

Prepared using General Method 2 and Purification Method 1. The product was then converted to the hydrochloride salt by the addition of 1 N hydrochloric acid, followed by evaporation.

LCMS (method B): Rt=9.32 min, m/z=436.14[M+H]⁺; C₂₉H₂₉N₃O, Mono-isotopic mass=435.23

¹H NMR (400 MHz, D₄-methanol) 6 7.99 (dd, J=9.5, 2.3 Hz, 1H), 7.90 (d, J=9.5 Hz, 1H), 7.61 (t, J=8.1 Hz, 1H), 7.41 (m, 2H), 7.27 (dd, J=7.9, 1.5 Hz, 1H), 7.20 (m, 2H), 7.16 (m, 2H), 7.10 (m, 2H), 4.46 (t, J=9.4 Hz, 2H), 3.41 (t, J=9.4 Hz, 2H), 3.287 (t, J=5.5 Hz, 4H), 2.63 (s, 3H), 1.88 (m, 4H), 1.70 (m, 2H).

### (j) 1-14-[2-(N,N-dimethylamino)ethoxy]phenyl}-4-methyl-8-phenoxy-2,3-dihydro-1H-pyrrolo[3,2-c]quinoline hydrochloride

Prepared using General Method 2 and Purification Method 1. The product was then converted to the hydrochloride salt by the addition of 1 N hydrochloric acid, followed by evaporation.

LCMS (method B): Rt=5.43 min, m/z=440.11 [M+H]⁺; C₂₈H₂₉N₃O₂, Mono-isotopic mass=439.23

¹H NMR (400 MHz, D₄-methanol) δ 7.81 (d, J=9.2 Hz, 1H), 9.57 (dd, J=9.2, 2.6 Hz, 1H), 7.32 (m, 4H), 7.20 (m, 1H), 7.01 (m, 2H), 6.85 (m, 2H), 6.49 (d, J=2.6 Hz, 1H), 4.39 (t, J=5.0 Hz, 2H), 4.30 (t, 9.4H, 2H), 3.70 (t, J=5.0 Hz, 2H), 3.36 (t, J=9.4 Hz, 2H), 3.03 (s, 6H), 2.61 (s, 3H).

### (k) 1-[4-(4-fluorophenoxy)phenyl]-8-methoxy-4-methyl-2,3-dihydro-1H-pyrrolo[3,2-c]quinoline hydrochloride

Prepared using General Method 2 and Purification Method 1. The product was then converted to the hydrochloride salt by the addition of 1 N hydrochloric acid, followed by evaporation.

LCMS (method B): Rt=8.43 min, m/z=401.06[M+H]⁺; C₂₅H₂₁FN₂O₂, Mono-isotopic mass=400.16

¹H NMR (400 MHz, D₄-methanol) δ 7.70 (d, J=9.4 Hz, 1H), 7.53 (m, 2H), 7.43 (dd, J=9.4, 2.7 Hz, 1H), 7.18 (m, 4H), 7.09 (m, 2H), 6.48 (d, J=2.7 Hz, 1H), 4.41 (t, J=9.6 Hz, 2H), 3047 (s, 3H), 3.40 (t, J=9.6 Hz, 2H), 2.61 (s, 3H).

### (l) 1-(benzodioxan-2-ylmethyl)-8-methoxy-4-methyl-2,3-dihydro-1H-pyrrolo[3,2-c]quinoline hydrochloride

Prepared using General Method 2 and Purification Method 1. The product was then converted to the hydrochloride salt by the addition of 1 N hydrochloric acid, followed by evaporation.

LCMS (method B): Rt=7.31 min, m/z=363.02[M+H]⁺; C₂₂H₂₂N₂O₃, Mono-isotopic mass=362.16

¹H NMR (400 MHz, D₄-methanol) δ 7.71 (d, J=9.3 Hz, 1H), 7.67 (d, J=2.6 Hz, 1H), 7.50 (dd J=9.3, 2.6 Hz, 1H), 6.88 (dd, J=8.0, 1.6 Hz, 1H), 6.82 (m, 1H), 6.76 (m, 1H), 6.63 (dd, J=8.0, 1.6 Hz, 1H), 4.81 (m, 1H), 4.46 (m, 2H), 4.24 (m, 3H), 4.12 (dd, J=15.8, 4.0 Hz, 1H), 3.83 (s, 3H), 3.25 (t, J=9.7 Hz, 2H), 2.53 (s, 3H).

### (m) 1-cyclohexyl-8-methoxy-4-methyl-2,3-dihydro-1H-pyrrolo[3,2-c]quinoline hydrochloride

Prepared using General Method 2 and Purification Method 1. The product was then converted to the hydrochloride salt by the addition of 1 N hydrochloric acid, followed by evaporation.

LCMS (method B): Rt=7.38 min, m/z=297.13[M+H]⁺; C₁₉H₂₄N₂O, Mono-isotopic mass=296.19

¹14 NMR (400 MHz, D₄-methanol) δ 7.70 (m, 1H), 7.52 (m, 2H), 4.54 (m, 1H), 4.14 (t, J=9.6 Hz, 2H), 3.96 (s, 3H), 3.16 (t, J=9.6 Hz, 2H), 2.48 (s, 3H), 2.12 (d, J=12.3 Hz, 2H), 1.98 (m, 2H), 1.80 (m, 3H), 1.54 (m, 2H), 1.32 (m, 1H).

### (n) 8-methoxy-4-methyl-1-phenyl-2,3-dihydro-1H-pyrrolo[3,2-c]quinoline hydrochloride

Prepared using General Method 2 and Purification Method 1. The product was then converted to the hydrochloride salt by the addition of 1 N hydrochloric acid, followed by evaporation.

LCMS (method B): Rt=6.75 min, m/z=291.08[M+H]⁺; C₁₉H₁₈N₂O, Mono-isotopic mass=290.14

¹H NMR (400 MHz, D₄-methanol) δ 7.72 (d, J=9.4 Hz, 1H), 7.64 (m, 2H), 7.56 (m, 3H), 7.40 (dd, J=9.4, 2.7 Hz, 1H), 6.38 (d, J=2.7 Hz, 1H), 4.45 (t, J=9.5 Hz, 2H), 3.42 (t, J=9.5 Hz, 2H), 3.33 (s, 3H), 2.62 (s, 3H).

### (o) 4-methyl-8-phenoxy-1-[4-(3-pyridyl)phenyl]-2,3-dihydro-1H-pyrrolo[3,2-c]quinoline hydrochloride

Prepared using General Method 2 and Purification Method 1. The product was then converted to the hydrochloride salt by the addition of 1 N hydrochloric acid, followed by evaporation.

LCMS (method B): Rt=7.48 min, m/z=446.05[M+H]⁺; C₂₉H₂₃N₃O₂, Mono-isotopic mass=445.18

¹H NMR (400 MHz, D₄-methanol) δ 8.80 (d, J=2.7 Hz, 1H), 8.67 (d, J=5.7 Hz, 1H), 8.24 (ddd, J=8.8, 2.7, 1.1 Hz, 1H), 8.09 (dd, J=8.8, 5.7 Hz, 1H), 7.86 (d, J=9.2 Hz, 1H), 7.64 (dd, J=9.2, 2.6 Hz, 1H), 7.48 (m, 2H), 7.40 (m, 2H), 7.28 (m, 1H), 7.23 (m, 2H), 6.91 (m, 2H), 6.47 (d, J=2.6 Hz, 1H), 4.38 (t, J=9.5 Hz, 3.41 (t, J=9.5 Hz, 2H), 2.64 (s, 3H).

### (p) 4-methyl-8-phenoxy-1-[2-(3-pyridyl)ethyl]-2,3-dihydro-1H-pyrrolo[3,2-c]quinoline hydrochloride

Prepared using General Method 2 and Purification Method 1. The product was then converted to the hydrochloride salt by the addition of 1 N hydrochloric acid, followed by evaporation.

LCMS (method B): Rt=5.48 min, m/z=382.13[M+H]⁺; C₂₅H₂₃N₃O, Mono-isotopic mass=381.18

¹H NMR (400 MHz, D₄-methanol) δ 8.78 (d, J=5.9 Hz, 1H), 8.75 (d, J=1.5 Hz, 1H), 8.32 (dt, J=8.1, 1.5 Hz, 1H), 8.02 (dd, J=8.1, 5.9 Hz, 1H), 7.86 (d, J=9.2 Hz, 1H), 7.66 (dd, J=9.2, 2.4 Hz, 1H), 7.56 (d, J=2.4 Hz, 1H), 7.41 (m, 2H), 7.15 (m, 3H), 4.12 (m, 4H), 3.25 (m, 4H), 2.55 (s, 3H).

### (q) 4-methyl-8-phenoxy-1-(2-pyridylmethyl)-2,3-dihydro-1H-pyrrolo[3,2-c]quinoline hydrochloride

Prepared using General Method 2 and Purification Method 1. The product was then converted to the hydrochloride salt by the addition of 1 N hydrochloric acid, followed by evaporation.

LCMS (method B): Rt=6.72 min, m/z=368.10[M+H]⁺; C₂₄H₂₁N₃O, Mono-isotopic mass=367.17

¹H NMR (400 MHz, D₄-methanol) δ 8.75 (dd, J=5.8, 1.2 Hz, 1H), 8.43 (m, 1H), 7.94 (m, 1H), 7.91 (d, J=9.2 Hz, 1H), 7.83 (d, J=8.0 Hz, 1H), 7.68 (dd, J=9.2, 2.5 Hz., 1H), 7.30 (m, 2H), 7.18 (m, 1H), 6.90 (m, 2H), 6.88 (d, J=2.5 Hz, 1H), 5.33 (s, 2H), 4.22 (t, J=9.5 Hz, 2H), 3.37 (t, J=9.5 Hz, 2H), 2.64 (s, 3H).

### (r) 4-methyl-1-(5-methylpyrazin-2-ylmethyl)-8-phenoxy-2,3-dihydro-1H-pyrrolo[3,2-c]quinoline hydrochloride

Prepared using General Method 2 and Purification Method 1. The product was then converted to the hydrochloride salt by the addition of 1 N hydrochloric acid, followed by evaporation.

LCMS (method B): Rt=6.51 min, m/z=383.10[M+H]⁺; C₂₄H₂₂N₄O, Mono-isotopic mass=382.18

¹H NMR (400 MHz, D₄-methanol) δ 8.50 (s, 1H), 8.43 (s, 1H), 7.83 (d, J=9.2 Hz, 1H), 7.62 (dd, J=9.2, 2.4 Hz, 1H), 7.30 (m, 3H), 7.15 (m, 1H), 6.92 (m, 2H), 5.13 (s, 2H), 4.20 (t, J=9.5 Hz, 2H), 3.29 (t, J=9.5 Hz, 2H), 2.62 (s, 3H), 2.57 (s, 3H).

### (s) 8-chloro-4-methyl-1-(2-phenylethyl)-2,3-dihydro-1H-pyrrolo[3,2-c]quinoline hydrochloride

Prepared using General Method 2 and Purification Method 1. The product was then converted to the hydrochloride salt by the addition of 1 N hydrochloric acid, followed by evaporation.

LCMS (method B): Rt=7.55 min, m/z=323.05[M+H]⁺; C₂₀H₁₉Cl N₂, Mono-isotopic mass=322.12.

¹H NMR (400 MHz, D₆-DMSO) δ 14.02 (s, 1H), 8.01 (d, J=2.2 Hz, 1H), 7.98 (d, J=9.2 Hz, 1H), 7.88 (dd, J=9.2, 2.2 Hz, 1H), 7.31 (m, 4H), 7.20 (m, 1H), 4.15 (t, J=7.3 Hz, 2H), 4.08 (t, J=9.6 Hz, 2H), 3.10 (m, 4H), 2.49 (s, 3H).

### (t) methyl 4-methyl-1-(2-phenylethyl)-2,3-dihydro-1H-pyrrolo[3,2-c]quinoline-8-carboxylate hydrochloride

Prepared using General Method 2 and Purification Method 1. The product was then converted to the hydrochloride salt by the addition of 1 N hydrochloric acid, followed by evaporation.

LCMS (method B): Rt=7.17 min, m/z=347.08[M+H]⁺; C₂₂H₂₂N₂O₂, Mono-isotopic mass=346.17

¹H NMR (400 MHz, D₆-DMSO) δ 14.09 (s, 1H), 8.78 (d, J=1.6 Hz, 1H), 8.29 (dd, J=8.9, 1.6 Hz, 1H), 8.05 (d, J=8.9 Hz, 1H), 7.45 (m, 2H), 7.36 (m, 2H), 7.25 (m, 1H), 4.19 (t, J=9.5 Hz, 2H), 4.12 (t, J=8.0 Hz, 2H), 3.95 (s, 3H), 3.16 (m, 4h), 2.52 (s, 3H).

### (u) 4-methyl-8-(morpholin-1-yl)-1-(2-phenylethyl)-2,3-dihydro-1H-pyrrolo[3,2-c]quinoline hydrochloride;

Prepared using General Method 2 and Purification Method 1. The product was then converted to the hydrochloride salt by the addition of 1 N hydrochloric acid, followed by evaporation.

LCMS (method B): Rt=7.26 min, m/z=374.14[M+H]⁺; C₂₄H₂₇N₃O, Mono-isotopic mass=373.22

¹H NMR (400 MHz, D₆-DMSO) δ 13.66 (s, 1H), 7.85 (d, J=9.5 Hz, 1H), 7.73 (dd, J=9.5, 2.4 Hz, 1H), 7.32 (m, 4H), 7.24 (m, 1H), 7.19 (d, J=2.4 Hz, 1H), 4.16 (t, J=7.5 Hz, 2H), 4.00 (t, J=9.7 Hz, 2H), 3.75 (m, 4H), 3.12 (m, 8H), 2.47 (s, 3H).

### (v) ethyl [4-methyl-1-(2-phenylethyl)-2,3-dihydro-1H-pyrrolo[3,2-c]quinoline-8-yl]acetate hydrochloride

Prepared using General Method 2 and Purification Method 1. The product was then converted to the hydrochloride salt by the addition of 1 N hydrochloric acid, followed by evaporation.

LCMS (method B): Rt=7.57 min, m/z=375.08[M+H]⁺; C₂₄H₂₆N₂O₂, Mono-isotopic mass=374.20

¹H NMR (400 MHz, D₆-DMSO) δ 13.67 (s, 1H), 8.05 (d, J=1.6 Hz, 1H), 7.88 (d, J=8.8 Hz, 1H), 7.76 (dd, J=8.8, 1.6 Hz, 1H), 7.34 (m, 2H), 7.29 (m, 2H), 7.21 (m, 1H), 4.16 (t, J=7.5 Hz, 2H), 4.11 (t, J=7.1 Hz, 2H), 4.03 (t, J=9.5 Hz, 2), 3.89 (s, 2H),. 3.09 (m, 4H), 2.48 (s, 3H), 1.19 (t, J=7.1 Hz, 3H).

### (w) 1-[3-(4-methyl-8-phenoxy-2,3-dihydro-1H-pyrrolo[3,2-c]quinolin-1-yl)propyl]-pyrrolidin-2-one hydrochloride

Prepared using General Method 2 and Purification Method 1. The product was then converted to the hydrochloride salt by the addition of 1 N hydrochloric acid, followed by evaporation.

LCMS (method B): Rt=6.58 min, m/z=402.11[M+H]⁺; C₂₅H₂₇N₃O₂, Mono-isotopic mass=401.21

¹H NMR (400 MHz, D₄-methanol) δ 7.80 (d, J=9.3 Hz, 1H), 7.61 (dd, J=9.3, 2.5 Hz, 1H), 7.51 (d, J=2.5 Hz, 1H), 7.47 (m, 2H), 7.25 (m, 1H), 7.14 (m, 2H), 4.14 (t, J=9.6 Hz, 2H), 3.72 (t, J=7.8 Hz, 2H), 3.42 (t, J=7.1 Hz, 2H), 3.21 (m, 4H), 2.51 (s, 3H), 2.35 (t, J=8.1 Hz, 2H), 2.03 (m, 2H), 1.90 (m, 2H).

### (x) 4-methyl-8-phenoxy-1-[2-(2-pyridyl)ethyl]-2,3-dihydro-1H-pyrrolo[3,2-c]quinoline hydrochloride

Prepared using General Method 2 and Purification Method 1. The product was then converted to the hydrochloride salt by the addition of 1 N hydrochloric acid, followed by evaporation.

LCMS (method B): Rt=6.28 min, m/z=382.10[M+H]⁺; C₂₅H₂₃N₃O, Mono-isotopic mass=381.18

¹H NMR (400 MHz, D₄-methanol) δ 8.78 (dd, J=5.9, 1.3 Hz, 1H), 8.54 (td, J=8.0, 1.3 Hz, 1H), 7.99 (m, 1H), 7.86 (d, J=9.4 Hz, 1H), 7.79 (d, J=8.0 Hz, 1H), 7.65 (dd, J=9.4, 2.5 Hz, 1H), 7.52 (d, J=2.5 Hz, 1H), 7.41 (m, 2H), 7.16 (m, 3H), 4.26 (t, J=7.3 Hz, 2H), 4.07 (t, J=9.6 Hz, 2H), 3.48 (t, J=7.3 Hz, 2H), 3.23 (t, J=9.6 Hz, 2H), 2.56 (s, 3H).

### (y) ethyl 3-(8-methoxy-4-methyl-2,3-dihydro-1H-pyrrolo[3,2-c]quinoline-1-yl)propionate hydrochloride

Prepared using General Method 2 and Purification Method 1. The product was then converted to the hydrochloride salt by the addition of 1 N hydrochloric acid, followed by evaporation.

LCMS (method B): Rt=6.21 min, m/z=315.07[M+H]⁺; C₁₈H₂₂N₂O₃, Mono-isotopic mass=314.16

¹H NMR (400 MHz, D₆-DMSO) δ 13.77 (s, 1H), 7.91 (d, J=9.3 Hz, 1H), 7.57 (dd, J=9.3, 2.5 Hz, 1H), 7.50 (d, J=2.5 Hz, 1H), 4.19 (t, J=7.3 Hz, 2H), 4.09 (m, 4H), 3.91 (s, 3H), 3.12 (t, J=9.6 Hz, 2H), 2.91 (t, J=7.3 Hz, 2H), 2.48 (s, 3H), 1.17 (t, J=7.2 Hz, 3H).

### (z) ethyl 4-(4-methyl-8-phenoxy-2,3-dihydro-1H-pyrrolo[3,2-c]quinoline-1-yl)butanoate hydrochloride

Prepared using General Method 2 and Purification Method 1. The product was then converted to the hydrochloride salt by the addition of 1 N hydrochloric acid, followed by evaporation.

LCMS (method B): Rt=7.67 min, m/z=391.07[M+H]⁺; C₂₄H₂₆N₂O₃, Mono-isotopic mass=390.19

¹H NMR (400 MHz, CD₃CN) δ 14.80 (s, 1H), 8.37 (d, J=9.3 Hz, 1H), 7.54 (dd, J=9.3, 2.5 Hz, 1H), 7.47 (d, J=2.5 Hz, 1H), 7.43 (m, 2H), 7.22 (m, 1H), 7.12 (m, 2H), 4.06 (q, J=7.1 Hz, 2H), 4.02 (t, J=9.6 Hz, 2H), 3.60 (m, 2H), 3.09 (t, J=9.6 Hz, 2H), 2.55 (s, 3H), 2.14 (t, J=7.3 Hz, 2H), 1.85 (m, 2H), 1.19 (t, J=7.1 Hz, 3H).

### (aa) methyl 4-(4-methyl-8-phenoxy-2,3-dihydro-1H-pyrrolo[3,2-c]quinoline-1-yl)butanoate hydrochloride

Prepared using General Method 2 and Purification Method 1. The product was then converted to the hydrochloride salt by the addition of 1 N hydrochloric acid, followed by evaporation.

LCMS (method B): Rt=7.26 min, m/z=377.08[M+H]⁺; C₂₃H₂₄N₂O₃, Mono-isotopic mass=376.18

¹H NMR (400 MHz, D₄-methanol) δ 7.81 (d, J=9.3 Hz, 1H), 7.65 (dd, J=9.3, 2.5 Hz, 1H), 7.53 (d, J=2.5 Hz, 1H), 7.46 (m, 2H), 7.25 (m, 1H), 7.14 (m, 2H), 4.12 (t, J=9.6 Hz, 2H), 3.67 (m, 2H), 3.65 (s, 3H), 3.19 (t, J=9.6 Hz, 2H), 2.50 (s, 3H), 2.18 (t, J=7.2 Hz, 2H), 1.90 (m, 2H).

### (ab) ethyl (4-methyl-8-phenoxy-2,3-dihydro-1H-pyrrolo[3,2-c]quinoline-1-yl)acetate hydrochloride

Prepared using General Method 2 and Purification Method 1. The product was then converted to the hydrochloride salt by the addition of 1 N hydrochloric acid, followed by evaporation.

LCMS (method B): Rt=7.21 min, m/z=362.99[M+H]⁺; C₂₂H₂₂N₂O₃, Mono-isotopic mass=362.16

¹H NMR (400 MHz, CD₃CN) δ 14.92 (s, 1H), 8.40 (d, J=9.3 Hz, 1H), 7.54 (dd, J=9.3, 2.5 Hz, 1H), 7.43 (m, 2H), 7.29 (d, J=2.5 Hz, 1H), 7.22 (m, 1H), 7.06 (m, 2H), 4.46 (s, 2H), 4.04 (m, 4H), 3.15 (t, J=9.6 Hz, 2H), 2.60 (s, 3H), 1.13 (t, J=7.0 Hz, 3H).

### (ac) 4-methyl-1-(1-methylpiperidin-4-yl)-8-phenoxy-2,3-dihydro-1H-pyrrolo[3,2-c]quinoline dihydrochloride

Prepared using General Method 2 and Purification Method 1. The product was then converted to the hydrochloride salt by the addition of 1 N hydrochloric acid, followed by evaporation.

LCMS (method B): Rt=4.68 min, m/z=374.14[M+H]⁺; C₂₄H₂₇N₃O, Mono-isotopic mass=373.22

¹H NMR (400 MHz, D₆-DMSO) δ 14.04 (s, 1H), 11.05 (s, 1H), 8.06 (d, J=9.2 Hz, 1H), 7.67 (m, 2H), 7.49 (m, 2H), 7.26 (m, 1H), 7.15 (m, 2H), 4.63 (m, 1H), 4.01 (t, J=9.6 Hz, 2H), 3.43 (d, J=12.0 Hz, 2H), 3.14 (t, J=9.6 Hz, 2H), 2.85 (m, 2H), 2.69 (s, 3H), 2.52 (s, 3H), 2.31 (m, 2H), 2.00 (d, J=13.0 Hz, 2H).

### (ad) 1-(1-benzylpyrrolidin-3-yl)-8-methoxy-4-methyl-2,3-dihydro-1H-pyrrolo[3,2-c]quinoline hydrochloride

Prepared using General Method 2 and Purification Method 1. The product was then converted to the hydrochloride salt by the addition of 1 N hydrochloric acid, followed by evaporation.

LCMS (method B): Rt=4.54 min, m/z=374.16[M+H]⁺; C₂₄H₂₇N₃O, Mono-isotopic mass=373.22

¹H NMR (400 MHz, D₆-DMSO+TFA-D) δ 7.90 (br d, J=9.0 Hz, 1H), 7.70-7.58 (m, 3H), 7.56-7.40 (m, 4H), 5.80-5.55 (m, 1H), 4.50 (2br s, 2H), 4.16 (m, 2H), 3.98 (2br s, 3H), 3.73 (m, 1H),3.61 (m, 2H), 3.43 (m, 1H), 3.17 (m, 2H), 2.51 (s, 3H), 2.48 (m, 2H).

### (ae) methyl 3-(4-methyl-8-phenoxy-2,3-dihydro-1H-pyrrolo[3,2-c]quinoline-1-yl)propionate hydrochloride

Prepared using General Method 2 and Purification Method 1. The product was then converted to the hydrochloride salt by the addition of 1 N hydrochloric acid, followed by evaporation.

LCMS (method B): Rt=7.05 min, m/z=363.07[M+H]⁺; C₂₂H₂₂N₂O₃, Mono-isotopic mass=362.16

¹H NMR (400 MHz, D₄-methanol) δ 7.83 (d, J=9.3 Hz, 1H), 7.65 (dd, J=9.3, 2.5 Hz, 1H), 7.54 (d, J=2.5 Hz, 1H), 7.45 (m, 2H), 7.24 (m, 1H), 7.14 (m, 2H), 4.13 (t, J=9.6 Hz, 2H), 3.97 (t, J=7.1 Hz, 2H), 3.65 (s, 3H), 3.18 (t, J=9.6 Hz, 2H), 2.66 (t, J=7.1 Hz, 2H), 2.52 (s, 3H).

### (af) 1-((S)-indan-1-yl)-4-methyl-8-phenoxy-2,3-dihydro-1H-pyrrolo[3,2-c]quinoline

Prepared using General Method 2 and Purification Method 3. The product was isolated as the acetate salt and then converted to the free base by partitioning between aqueous sodium carbonate and dichloromethane followed by evaporation of the organic phase.

LCMS (method B): Rt=8.81 min, m/z=393.06[M+H]⁺; C₂₇H₂₄N₂O, Mono-isotopic mass=392.19

¹H NMR (400 MHz, CDCl₃) δ 7.97 (d, J=9.2 Hz, 1H), 7.64 (d, J=2.6 Hz, 1H), 7.31 (m, 3H), 7.24 (m, 2H), 7.16 (m, 2H), 7.08 (m, 1H), 7.02 (m, 2H), 5.88 (t, J=7.6 Hz, 1H), 3.46 (m, 2H), 3.00 (m, 3H), 2.85 (m, 1H), 2.52 (s, 3H), 2.32 (m, 1H), 2.11 (m, 1H).

### (ag) 1-((R)-indan-1-yl)-4-methyl-8-phenoxy-2,3-dihydro-1H-pyrrolo[3,2-c]quinoline

Prepared using General Method 2 and Purification Method 3. The product was isolated as the acetate salt and then converted to the free base by partitioning between aqueous sodium carbonate and dichloromethane followed by evaporation of the organic phase.

LCMS (method B): Rt=8.68 min, m/z=393.11[M+H]⁺; C₂₇H₂₄N₂O, Mono-isotopic mass=392.19

¹H NMR (400 MHz, CDCl₃) δ 7.98 (d, J=9.2 Hz, 1H), 7.64 (d, J=2.6 Hz, 1H), 7.31 (m, 3H), 7.24 (m, 2H), 7.16 (m, 2H), 7.08 (m, 1H), 7.02 (m, 2H), 5.87 (t, J=7.6 Hz, 1H), 3.45 (m, 2H), 2.99 (m, 3H), 2.84 (m, 1H), 2.52 (s, 3H), 2.32 (m, 2H), 2.11 (m, 1H).

### (ah) 1-(3-methoxypropyl)-4-methyl-8-phenoxy-2,3-dihydro-1H-pyrrolo[3,2-c]quinoline hydrochloride

Prepared using General Method 2 and Purification Method 1. The product was then converted to the hydrochloride salt by the addition of 1 N hydrochloric acid, followed by evaporation.

LCMS (method B): Rt=7.31 min, m/z=349.13[M+H]⁺; C₂₂H₂₄N₂O₂, Mono-isotopic mass=348.18

¹H NMR (400 MHz, D₆-DMSO) δ 13.76 (s, 1H), 8.00 (m, 1H), 7.68 (m, 2H), 7.46 (m, 2H), 7.23 (m, 1H), 7.12 (m, 2H), 4.06 (t, J=9.6 Hz, 2H), 3.73 (t, J=7.5 Hz, 2H), 3.22 (t, J=5.8 Hz, 2H), 3.14 (s, 3H), 3.13 (t, J=9.6 Hz, 2H), 2.49 (s, 3H), 1.82 (m, 2H).

### (ai) 4-methyl-8-phenoxy-1-(tetrahydrofuran-2-ylmethyl)-2,3-dihydro-1H-pyrrolo[3,2-c]quinoline hydrochloride

Prepared using General Method 2 and Purification Method 1. The product was then converted to the hydrochloride salt by the addition of 1 N hydrochloric acid, followed by evaporation.

LCMS (method B): Rt=7.35 min, m/z=361.12[M+H]⁺; C₂₃H₂₄N₂O₂, Mono-isotopic mass=360.16

¹H NMR (400 MHz, D₆-DMSO) δ 13.83 (s, 1H), 8.01 (d, J=9.2 Hz, 1H), 7.71 (dd, J=9.2, 2.5 Hz, 1H), 7.68 (d, J=2.5 Hz, 1H), 7.48 (m, 2H), 7.25 (m, 1H), 7.16 (m, 2H), 4.15 (td, J=11.3, 7.9 Hz, 1H), 4.03 (m, 2H), 3.73 (d, J=6.0 Hz, 2H), 3.52 (t, J=6.7 Hz, 2H), 3.13 (m, 2H), 2.50 (s, 3H), 1.74 (m, 3H), 1.27 (m, 1H).

### (aj) 1-[2-(4-chlorophenyl)ethyl]-4-methyl-8-phenoxy-2,3-dihydro-1H-pyrrolo[3,2-c]quinoline hydrochloride

Prepared using General Method 2 and Purification Method 1. The product was then converted to the hydrochloride salt by the addition of 1 N hydrochloric acid, followed by evaporation.

LCMS (method B): Rt=8.91 min, m/z=415.08[M+H]⁺; C₂₆H₂₃ClN₂O, Mono-isotopic mass=414.15.

¹H NMR (400 MHz, D₆-DMSO) δ 13.82 (2s, 1H), 8.01 (m, 1H), 7.68 (dd, J=9.2, 2.5 Hz, 1H), 7.54 (d, J=2.5 Hz, 1H), 7.45 (m, 2H), 7.26 (m, 2H), 7.21 (m, 1H), 7.14 (m, 2H), 7.06 (m, 2H), 3.98 (t, J=9.6 Hz, 2H), 3.92 (t, J=7.5 Hz, 2H), 3.10 (t, J=9.6 Hz, 2H), 2.90 (t, J=7.5 Hz, 2H), 2.50 (s, 3H).

### (ak) 1-[2-(4-methoxyphenyl)ethyl]-4-methyl-8-phenoxy-2,3-dihydro-1H-pyrrolo[3,2-c[quinoline hydrochloride

Prepared using General Method 2 and Purification Method 1. The product was then converted to the hydrochloride salt by the addition of 1 N hydrochloric acid, followed by evaporation.

LCMS (method B): Rt=8.44 min, m/z=411.12[M+H]⁺; C₂₇H₂₆N₂O₂, Mono-isotopic mass=410.20

¹H NMR (400 MHz, D₆-DMSO) δ 13.67 (s, 1H), 7.97 (d, J=9.3 Hz, 1H), 7.69 (dd, J=9.3, 2.5 Hz, 1H), 7.56 (d, J=2.5 Hz, 1H), 7.46 (m, 2H), 7.23 (m, 1H), 7.15 (m, 2H), 6.93 (m, 2H), 6.76 (m, 2H), 3.97 (t, J=9.6 Hz, 2H), 3.87 (t, J=7.4 Hz, 3.69 (s, 3H), 3.09 (t, J=9.6 Hz, 2H), 2.83 (t, J=7.4 Hz, 2H), 2.49 (s, 3H).

### (al) 4-methyl-8-phenoxy-1-(2-phenylpropyl)-2,3-dihydro-1H-pyrrolo[3,2-c]quinoline hydrochloride

Prepared using General Method 2 and Purification Method 1. The product was then converted to the hydrochloride salt by the addition of 1 N hydrochloric acid, followed by evaporation.

LCMS (method B): Rt=8.72 min, m/z=395.11[M+H]⁺; C₂₇H₂₆N₂O, Mono-isotopic mass=394.20

¹H NMR (400 MHz, D₆-DMSO) δ 13.81 (s, 1H), 8.01 (d, J=9.3 Hz, 1H), 7.73 (dd, J=9.3, 2.5 Hz, 1H), 7.51 (m, 2H), 7.44 (d, J=2.5 Hz, 1H), 7.28 (m, 1H), 7.22 (m, 5H), 7.09 (m, 2H), 3.92 (m, 2H), 3.67 (dd, J=15.0, 9.4 Hz, 1H), 3.38 (m, 1H), 3.04 (m, 2H), 2.88 (m, 1H), 2.46 (s, 3H), 1.04 (d, J=7.0 Hz, 3H).

### (am) 8-cyano-4-methyl-1-(2-phenylethyl)-2,3-dihydro-1H-pyrrolo[3,2-c]quinoline hydrochloride

Prepared using General Method 2 and Purification Method 1. The product was then converted to the hydrochloride salt by the addition of 1 N hydrochloric acid, followed by evaporation.

LCMS (method B): Rt=6.59 min, m/z=314.12[M+H]⁺; C₂₁H₁₉N₃, Mono-isotopic mass=313.16

¹H NMR (400 MHz, D₆-DMSO) δ 14.21 (s, 1H), 8.45 (d, J=1.6 Hz, 1H), 8.11 (dd, J=8.9, 1.6 Hz, 1H), 8.05 (d, J=8.9 Hz, 1H), 7.34 (m, 2H), 7.26 (m, 2H), 7.17 (m, 1H), 4.25 (t, J=7.1 Hz, 2H), 4.10 (t, J=9.6 Hz, 2H), 3.12 (m, 4H), 2.50 (s, 3H).

### (an) 8-hydroxy-4-methyl-1-(2-phenylethyl)-2,3-dihydro-1H-pyrrolo[3,2-c]quinoline hydrochloride

Prepared using General Method 2 and Purification Method 1. The product was then converted to the hydrochloride salt by the addition of 1 N hydrochloric acid, followed by evaporation.

LCMS (method B): Rt=6.75 min, m/z=305.11[M+H]⁺; C₂₀H₂₀N₂O, Mono-isotopic mass=304.16

¹H NMR (400 MHz, D₆-DMSO) δ 13.51 (s, 1H), 10.44 (s, 1H), 7.82 (d, J=9.2 Hz, 1H), 7.57 (d, J=2.3 Hz, 1H), 7.44 (dd, J=9.2, 2.3 Hz, 1H), 7.33 (m, 4H), 7.24 (m, 1H), 4.07 (t, J=7.4 Hz, 2H), 3.90 (t, J=9.6 Hz, 2H), 3.08 (t, J=7.4 Hz, 2H), 3.04 (t, J=9.6 Hz, 2H), 2.45 (s, 3H).

### (ao) 8-phenoxy-1-(2-phenylethyl)-2,3-dihydro-1H-pyrrolo[3,2-c]quinoline hydrochloride

### Prepared using General Method 3 and Purification Method 4. The product was then converted to the hydrochloride salt by the addition of 1 N hydrochloric acid, followed by evaporation.

### LCMS (method B): Rt=8.52 min, m/z=367.08[M+H]⁺; C₂₅H₂₂N₂O, Mono-isotopic mass=366.17

¹H NMR (400 MHz, D₆-DMSO) δ 13.79 (s, 1H), 8.26 (s, 1H), 7.98 (d, J=9.3 Hz, 1H), 7.72 (dd, J=9.3, 2.5 Hz, 1H), 7.60 (d, J=2.5 Hz, 1H), 7.46 (m, 2H), 7.19 (m, 6H), 7.03 (m, 2H), 3.99 (t, J=9.5 Hz, 2H), 3.94 (t, J=7.6 Hz, 2H), 3.16 (t, J=9.5 Hz, 2H), 2.91 (t, J=7.6 Hz, 2H).

### Preparative Example 5

### 6,8-Dimethoxy-1-(4-hydroxyphenyl)-4-methylpyrrolo[3,2-c]quinoline trifluoroacetate

A mixture of 6,8-dimethoxy-1-(4-hydroxyphenyl)-4-methyl-2,3-dihydro-1H-pyrrolo[3,2-c]quinoline (0.1 g; see Preparative Example 3(i) above), palladium on carbon (10%, 0.1 g) and diphenyl ether (5 mL) was heated at 200° C. for 2 hours. The mixture was cooled to room temperature, diluted with methanol and filtered through Celite™. The filtrate was evaporated to dryness and the residue was purified using Purification Method 1 to give the title compound (0.004 g).

LCMS (Method B): Rt=6.50 min, m/z=335.13 [M+H]⁺; C₂₀H₁₈N₂O₃, Mono-isotopic mass=334.13

¹H-NMR (400 MHz, D₆-DMSO): δ 10.24 (s, br, 1H), 7.92 (d, J=3.3 Hz, 1H), 7.45 (m, 2H), 7.44 (d, J=3.3 Hz, 1H), 7.08 (d, J=2.4 Hz, 1H), 7.06 (m, 2H), 6.25 (d, J=2.4 Hz, 1H), 4.13 (s, 3H), 3.52 (s, 3H), 3.15 (s, 3H).

### Preparative Example 6

### 8-Methoxy-4-methyl-1-[4-(4-methylpiperazin-1-yl)-3-fluorophenyl]-2,3-dihydro-1H-pyrrolo[3,2-c]quinoline hydrochloride

### (i) 1-(4-Bromo-3-fluorophenyl)-8-methoxy-4-methyl-2,3-dihydro-1H-pyrrolo[3,2-c]quinoline

The sub-title compound was prepared from the appropriate intermediates by analogy with General Method 3 (above) and was purified using Purification Method 1.

¹H NMR (400 Mz, D₄-methanol) δ 7.86 (t, J=8.1 Hz, 1H), 7.77 (d, J=9.3 Hz, 1H), 7.51 (dd, J=9.2, 2.4 Hz, 1H), 7.47 (dd, J=9.3, 2.7 Hz, 1H), 7.31 (m, 1H), 6.42 (d, J=2.7 Hz, 1H), 4.45 (t, J=9.3 Hz, 2H), 3.47 (s, 3H), 3.42 (t, J=9.3 Hz, 2H), 2.64 (s, 3H).

The compound was converted to the free base by partitioning between dichloromethane and aqueous sodium bicarbonate solution, followed by evaporation of the organic phase. The free base was used directly without further purification.

### (ii) 8-Methoxy-4-methyl-1-[4-(4-methylpiperazin-1-yl)-3-fluorophenyl]-2,3-dihydro-1H-pyrrolo[3,2-e]quinoline hydrochloride

A mixture of 1-(4-bromo-3-fluorophenyl)-8-methoxy-4-methyl-2,3-dihydro -1H-pyrrolo[3,2-e]quinoline (0.075 g; see step (i) above), 1-methylpiperazine (0.023 g), palladium acetate (0.003 g), 2-(di-tert-butylphosphino)biphenyl (0.003 g), sodium tert-butoxide (0.026 g) and toluene (5 mL) was stirred and heated at 80° C. under an atmosphere of nitrogen overnight. The mixture was then stirred and heated at reflux overnight. Further palladium acetate (0.003 g) and 2-(di-tert-butylphosphino)biphenyl (0.003 g) was added and the mixture was stirred and heated at reflux overnight. The mixture was evaporated to dryness and the residue was partitioned between ethyl acetate and aqueous sodium bicarbonate solution. The organic layer was washed with water, aqueous brine solution, dried (MgSO₄) and filtered. The filtrate was evaporated to dryness and the residue was purified using Purification Method 1. The product was then converted to the hydrochloride salt by the addition of 1 N hydrochloric acid, followed by evaporation to give the title compound (0.009 g).

LCMS (Method B): Rt=4.79 min, m/z=407.17 [M+H]⁺; C₂₄H₂₇FN₄O, Mono-isotopic mass=406.22

¹H-NMR (400 MHz, D₄-methanol): δ 7.75 (d, J=9.2 Hz, 1H), 7.45 (dd, J=9.2, 2.7 Hz, 1H), 7.42 (dd, J=12.9, 2.3 Hz, 1H), 7.37 (dd, J=8.4, 2.3 Hz, 1H), 7.34 (t, J=8.4 Hz, 1H), 6.45 (d, J=2.7 Hz, 1H), 4.40 (t, J=9.5 Hz, 2H), 3.68 (s, br, 4H), 3.45 (s, 3H), 3.40 (t, J=9.5 Hz, 2H), 3.40, 3.25 (broad singlets, 4H), 3.00 (s, 3H), 2.62 (s, 3H).

### Preparative Example 7

### 4-Methyl-8-phenylamino-1-(2-phenylethyl)-2 3-dihydro-1H-pyrrolo[3 2-c]quinoline formate

### (i) 8-Bromo-4-methyl-1-(2-phenylethyl)-2,3-dihydro-1H-pyrrolo[3,2-c]quinoline

The sub-title compound was prepared from the appropriate intermediates by analogy with General Method 3 (above) and then used without purification.

LCMS (method A): Rt=2.42 min, m/z=367 [M+H]⁺; C₂₀H₁₉BrN₂, Mono-isotopic mass=367.07

### (ii) 4-Methyl-8-phenylamino-1-(2-phenylethyl)-2,3-dihydro-1H-pyrrolo[3,2-c]quinoline formate

A mixture of 8-bromo-4-methyl-1-(2-phenylethyl)-2,3-dihydro-1H-pyrrolo[3,2-c]quinoline (0.308 g; see step (i) above), aniline (0.064 mL) 2-dicyclohexyl-phosphino 2′-dimethylamino biphenyl (0.028 g), tris-(dibenzylidieneacetone)-dipalladium (0.032 g), sodium tert-butoxide (0.094 g) and toluene (8 mL) was degassed and then heated in the microwave at 140° C. for 30 minutes. The mixture was diluted with water, extracted with ethyl acetate, washed with water, dried (MgSO₄) and filtered. The filtrate was evaporated to dryness and the residue was purified using Purification Method 1 to give the title compound (0.08 g).

LCMS (method B): Rt=8.61 min, m/z=380.12[M+H]⁺; C₂₆H₂₅N₃, Mono-isotopic mass=379.20

¹H NMR (400 MHz, D₆-DMSO) δ 8.40 (s, 1H), 8.27 (s, 1H), 7.70 (d, J=9.2 Hz, 1H), 7.66 (d, J=2.4 Hz, 1H), 7.35 (dd, J=9.2, 2.4 Hz, 1H), 7.23 (m, 2H), 7.17 (m, 5H), 7.06 (m, 2H), 6.87 (m, 1H), 3.73 (m, 4H), 3.00 (t, J=9.4 Hz, 2H), 2.88 (t, J=7.8 Hz, 2H), 2.38 (s, 3H).

### Preparative Example 8

### [4-Methyl-1-(2-phenylethyl)-2,3-dihydro-1H-pyrrolo[2,3-c]quinoline-8-oyl]piperidine hydrochloride

### (i) 4-Methyl-1-(2-phenyl ethyl)-2,3-dihydro-1H-pyrrolo[3,2-c]quinoline-8-carboxylic acid

Crude methyl 4-methyl-1-(2-phenylethyl)-2,3-dihydro-1H-pyrrolo[3,2-c]quinoline-8-carboxylate (see Preparative Example 4 (t) above) was dissolved in a mixture of methanol (3 mL) and water (3 mL) and sodium hydroxide (0.2 g) was added and the mixture was stirred at room temperature for 1 hour. The mixture was evaporated to dryness and the residue was dissolved in ethyl acetate and washed with aqueous citric acid solution, dried (MgSO₄) and filtered. The filtrate was evaporated to dryness and the residue was purified using Purification Method 1. The product (sub-title compound) was used directly without further purification.

### (ii) [4-Methyl-1-(2-phenylethyl)-2 3-dihydro-1H-pyrrolo[2 3-c]quinoline-8-oyl]piperidine hydrochloride

A mixture of crude 4-methyl-1-(2-phenylethyl)-2,3-dihydro-1H-pyrrolo[3,2-c]quinoline-8-carboxylic acid (0.05 g; see step (i) above), piperidine (0.085 g), ethyl acetate (2 mL), pyridine (0.2 mL) and O-(7-azabenzotriazol-1-yl)-N,N,N′,N′-tetramethyluronium hexafluorophosphate (0.051 g) was stirred at room temperature for 2 hours. The resultant mixture was evaporated to dryness and the residue was purified by Purification Method 1. The product was then converted to the hydrochloride salt by the addition of 1 N hydrochloric acid, followed by evaporation to give the title compound (0.023 g).

LCMS (method B): Rt=7.06 min, m/z=400.14[M+H]⁺; C₂₆H₂₉N₃O, Mono-isotopic mass=399.23

¹H NMR (400 MHz, D₆-DMSO) δ 13.88 (s, 1H), 8.08 (d, J=1.5 Hz, 1H), 7.99 (d, J=8.8 Hz, 1H), 7.85 (dd, J=8.8, 1.5 Hz, 1H), 7.32 (m, 4H), 7.23 (m, 1H), 4.13 (t, J=7.7 Hz, 2H), 4.07 (t, J=9.5 Hz, 2H), 3.59 (br, 2H), 3.31 (br, 2H), 3.12 (m, 4H), 2.51 (s, 3H), 1.50 (br m, 6H).

### Preparative Example 9

### 4-Methyl-1-(2-phenylethyl)-8-phenoxy-2,3-dihydro-1H-pyrrolo[3,2-c]quinoline hydrochloride

Large Scale Process Outline.

| Step | | | |
|---|---|---|---|
| No. | Operation | Charges | |
| 1 | Set-up 10 L flange flask and purge with N₂. | | |
| 2 | Charge to flask stage 4-chloro-3-(2-chloroethyl)- | 745.3 | g |
| | 2-methyl-6-phenoxyquinoline (1 eq.; see | | |
| | Preparation 4 above). | | |
| 3 | Charge to flask ethylene glycol (5 volumes). | 3700 | mL |
| 4 | Nitrogen was bubbled through the reaction | | |
| | mixture for 30 min. | | |
| 5 | Phenethylamine (2.0 eq) was added and the flask | 560 | mL |
| | evacuated and purged with N₂ 3x. | | |
| 6 | The reaction mixture was heated to 180° C. | | |
| | under an atmosphere of N₂. | | |
| 7 | The reaction was analysed for completion after | | |
| | 2 h by LC analysis and every hour thereafter until | | |
| | reaction complete. The reaction was deemed | | |
| | complete when the level of reaction intermediate | | |
| | at rt 16.8 min <1% . | | |
| 8 | The reaction mixture was allowed to cool to room | | |
| | temperature and left to stir overnight. | | |
| 9 | Water (5 volumes) was added to the reaction | 3700 | mL |
| | mixture. | | |
| 10 | The reaction mixture was acidified to pH 1 using | ~10 | mL |
| | conc. HCl. | | |
| 11 | The mixture was extracted using DCM (2 × 5 | 2 × 3700 | mL |
| | volumes). | | |
| 12 | The DCM extracts were combined and washed | 3700 | mL |
| | with water (5 volumes). | | |
| 13 | The DCM extracts were dried over MgSO₄. | | |
| 14 | The DCM mixture was distilled under | | |
| | atmospheric pressure until precipitate starts | | |
| | to form. | | |
| 15 | Acetone (6 volumes) was added and the | 4500 | mL |
| | remaining amount of DCM removed by | | |
| | distillation until a constant head temperature | | |
| | was reached. Add further amount of acetone | | |
| | to increase mobility of slurry if required. | | |
| 16 | The mixture was allowed to cool to room | | |
| | temperature and stirred for 1 h. | | |
| 17 | The precipitate was filtered and the solid cake | 1500 | mL |
| | washed with acetone (2 volumes). | | |
| 18 | The crude stage 3 was dissolved in EtOH | 3000 | mL |
| | (4 volumes) at reflux. | | |
| 19 | The mixture was allowed to cool to 70° C. | | |
| | and a polish and filter carried out transferring | | |
| | to mixture to a 10 L flange flask. | | |
| 20 | The filtered mixture was allowed to cool to room | 1500 | mL |
| | temperature resulting in the formation of a | | |
| | precipitate. The precipitate was filtered and | | |
| | the wet cake washed with acetone (2 volumes). A | | |
| | further recrystallisation can be used to improve | | |
| | purity. | | |
| 21 | The title compound was dried in a vacuum oven | | |
| | to constant weight at 50° C. | | |

### Working Examples

### Example 1

Compounds of Examples 1 to 9 above may be formulated for topical administration according to any of the following formulations (wherein “active compound” represents any of the compounds of Examples 1 to 9 above).

| Ingredient | Formulation | | | | | | | |
|---|---|---|---|---|---|---|---|---|
| (w/w) | 1(i) | 2(ii) | 3(i) | 4(ii) | 5(i) | 6(ii) | 7 | 8 |
| Benzyl alcohol | 1.0 | 1.0 | 1.0 | 1.0 | — | — | 2.0 | 2.1 |
| Potassium | — | — | — | — | 0.04 | 0.1 | — | — |
| sorbate | | | | | | | | |
| EDTA | — | — | — | — | 0.04 | 0.1 | — | — |
| Ethanol | 9.9 | 10 | 7.7 | 7.7 | — | — | 10.2 | 10 |
| Propylene | 15.2 | 15 | 5.7 | 5.7 | — | — | 15.0 | 14.9 |
| glycol | | | | | | | | |
| Glycerol | — | — | 25.1 | 25 | — | — | — | 10.2 |
| Kleptose ™ | — | — | — | — | 20.1 | 20 | — | — |
| Klucel ™ | — | — | — | — | 2.0 | 2.0 | — | — |
| HEC | 2 | 2 | 2.2 | 2.0 | — | — | 2 | 2.0 |
| Water | — | — | — | — | 75.82 | 75.8 | 68.8 | 58.8 |
| Citrate/ | 69.8 | 70 | 56.3 | 56.6 | — | — | — | — |
| phosphate | | | | | | | | |
| pH 5.5 buffer | | | | | | | | |
| Active | 2.0 | 2.0 | 2.0 | 2.0 | 2.0 | 2.0 | 2.0 | 2.0 |
| compound | | | | | | | | |

Alternative formulations include those based upon 2(i) and 2(ii) above, but having increased propylene glycol concentration (but less than 15% w/w) and decreased glycerol concentration.

### Example 2

The compounds of Preparative Example 9 above was formulated for topical administration according to the following formulations (wherein “active compound” represents 4-methyl-1-(2-phenylethyl)-8-phenoxy-2,3-dihydro-1H-pyrrolo[3,2-c]quinoline hydrochloride).

| Ingredient | Formulation | | | | | | |
|---|---|---|---|---|---|---|---|
| (w/w) | 9(D) | 9(P) | 10 | 11(D) | 11(P) | 12(D) | 12(P) |
| Benzyl alcohol | 1.01 | 1.00 | 1.0 | 0.99 | 0.99 | 1.0 | 1.0 |
| Ethanol | 8.01 | 7.97 | 8.0 | 4.99 | 5.01 | 8.0 | 8.0 |
| Propylene | 6.02 | 5.99 | 6.0 | 4.00 | 4.01 | 6.0 | 6.0 |
| glycol | | | | | | | |
| Glycerol | 20.05 | 19.94 | 20.0 | 14.94 | 15.00 | 20.0 | 20.0 |
| HEC | 1.99 | 2.05 | 2.0 | 2.08 | 1.97 | 2.0 | 2.0 |
| Citrate/ | — | — | — | — | — | 62.0 | 63.0 |
| phosphate | | | | | | | |
| pH 5.5 buffer | | | | | | | |
| Water | 60.90 | 63.05 | 62.0 | 72.5 | 73.01 | — | — |
| Active | 2.01 | — | 1.0 | 0.51 | — | 1.0 | — |
| compound | | | | | | | |

### Example 3

As depicted in FIGS. 1 to 3, compositions of Examples 1 and 2 above were, when applied to infected skin (in a mouse model) effective in killing microorganisms (bacteria and fungi). Indeed, compared to no treatment or mere application of the topical formulation base (without the compound of formula I), Formulation 9(D) of Example 2 above demonstrated a log kill of approximately 7.0 against S. aureus on intact mouse skin.

### Abbreviations

br=broad (in relation to NMR)

doublet (in relation to NMR)

DCM=dichloromethane

DMSO=dimethylsulfoxide

EDTA=ethylenediaminetetraacetic acid

HEC=hydroxyethylcellulose

HPLC=high performance liquid chromatography

LC=liquid chromatography

m=multiplet (in relation to NMR)

MBC=minimum bactericidal concentration

Me=methyl

min.=minute(s)

MIC=minimum inhibitory concentration

MS=mass spectroscopy

NMR=nuclear magnetic resonance

q=quartet (in relation to NMR)

s=singlet (in relation to NMR)

t=triplet (in relation to NMR)

Prefixes n-, s-, i-, t- and tert- have their usual meanings: normal, secondary, iso, and tertiary.

1. A topical pharmaceutical composition comprising a compound of formula I, or a pharmaceutically-acceptable derivative thereof, in admixture with a pharmaceutically acceptable adjuvant, diluent or carrier, wherein the compound of formula I has the following structure, 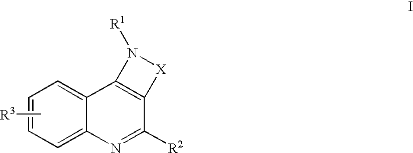
wherein
R¹ represents
(a) H,
(b) C₁₋₁₂ alkyl, C₃₋₁₂ cycloalkyl, C₃₋₁₂ cycloalkenyl (which latter three groups are optionally substituted by one or more substituents selected from halo, nitro, CN, C₁₋₆ alkyl, C₁₋₆ alkenyl, C₁₋₆ alkynyl, C₃₋₈ cycloalkyl (which latter three groups are optionally substituted by one or more substituents selected from OH, ═O, halo, C₁₋₄ alkyl and C₁₋₄ alkoxy), OR^{4a}, S(O)ₙR^{4b}, S(O)₂N(R^{4c})(R^{4d}), N(R^{4e})S(O)₂R^{4f}, N(R^{4g})(R^{4h}), B¹—C(O)—B²—R⁴ⁱ, aryl and Het¹, and which C₃₋₁₂ cycloalkyl or C₄₋₁₂ cycloalkenyl groups may additionally be substituted by ═O),
(c) aryl or
(d) Het²;
R² represents
(a) H,
(b) C₁₋₁₂ alkyl, C₁₋₁₂ alkenyl, C₁₋₁₂ alkynyl, C₃₋₁₂ cycloalkyl or C₄₋₁₂ cycloalkenyl, which latter five groups are optionally substituted by one or more substituents selected from halo, nitro, CN, C₁₋₆ alkyl, C₁₋₆ alkenyl, C₁₋₆ alkynyl, C₃₋₈ cycloalkyl (which latter three groups are optionally substituted by one or more substituents selected from OH, ═O, halo, C₁₋₄ alkyl and C₁₋₄ alkoxy), OR^{5a}, S(O)ₚR^{5b}, S(O)₂N(R^{5c})(R^{5d}), N(R^{5e})S(O)₂R^{5f}, N(^{5g})(R^{5h}), B³—C(O)—B⁴—R⁵ⁱ, aryl and Het³, and which C₃₋₁₂ cycloalkyl or C₄₋₁₂ cycloalkenyl groups may additionally be substituted by ═O,
(c) aryl or
(d) Het⁴;
R³ represents H or one to four substituents on the fused benzene ring selected from
(a) halo,
(b) CN,
(c) C₁₋₁₂ alkyl, C₁₋₁₂ alkenyl, C₁₋₁₂ alkynyl, C₃₋₁₂ cycloalkyl or C₄₋₁₂ cycloalkenyl, which latter five groups are optionally substituted by one or more substituents selected from halo, nitro, CN, C₁₋₆ alkyl, C₁₋₆ alkenyl, C₁₋₆ alkynyl, C₃₋₈ cycloalkyl (which latter three groups are optionally substituted by one or more substituents selected from OH, ═O, halo, C₁₋₄ alkyl and C₁₋₄ alkoxy), OR^{6a}, S(O)_{q}R^{6b}, S(O)₂N(R^{6c})(R^{6d}), N(R^{6e})S(O)₂R^{6f}, N(R^{6g})(R^{6h}), B⁵—C(O)—B⁶—R⁶ⁱ, aryl and Het⁵, and which C₃₋₁₂ cycloalkyl or C₄₋₁₂ cycloalkenyl groups may additionally be substituted by ═O,
(d) OR^{7a},
(e) S(O)ᵣR^{7b},
(f) S(O)₂N(R^{7c})(R^{7d}),
(g) N(R^{7e})S(O)₂R^{7f},
(h) N(R^{7g})(R^{7h}),
(i) B⁷—C(O)—B⁸—R⁷ⁱ,
(j) aryl or p1 (k) Het⁶;
R^{4a} to R⁴ⁱ, R^{5a} to R⁵ⁱ, R^{6a} to R⁶ⁱ and R^{7a} to R⁷ⁱ independently represent, at each occurrence,
(a) H,
(b) C₁₋₁₀ alkyl, C₂₋₁₀ alkenyl, C₂₋₁₀ alkynyl (which latter three groups are optionally substituted by one or more substituents selected from halo, OH, C₁₋₆ alkoxy, aryl and Het⁷),
(c) C₃₋₁₀ cycloalkyl, C₄₋₁₀ cycloalkenyl (which latter two groups are optionally substituted by one or more substituents selected from halo, OH, ═O, C₁₋₆ alkyl, C₁₋₆ alkoxy, aryl and Het⁸),
(d) aryl or
(e) Het⁹,
provided that R^{4b}, R^{5b}, R^{6b} or R^{7b} does not represent H when n, p, q or r, respectively is 1 or 2;
X represents
(a) —C(R^{8a})(R^{8b})—C(R^{8c})(R^{8d})— or
(b) —C(R^{8e})═C(R^{8f})—;
R^{8a} to R^{8f} independently represent H, halo or C₁₋₄ alkyl;
each aryl independently represents a C₆₋₁₀ carbocyclic aromatic group, which group may comprise either one or two rings and may be substituted by one or more substituents selected from
(a) halo,
(b) CN,
(c) C₁₋₁₂ alkyl, C₁₋₁₂ alkenyl, C₁₋₁₂ alkynyl, C₃₋₁₂ cycloalkyl or C₄₋₁₂ cycloalkenyl, which latter five groups are optionally substituted by one or more substituents selected from halo, nitro, CN, C₁₋₆ alkyl, C₁₋₆ alkenyl, C₁₋₆ alkynyl, C₃₋₈ cycloalkyl (which latter three groups are optionally substituted by one or more substituents selected from OH, ═O, halo, C₁₋₄ alkyl and C₁₋₄ alkoxy), OR^{9a}, S(O)^{t}R^{9b}, S(O)₂N(R^{9c})(R^{9d}), N(R^{9e})S(O)²R^{9f}, N(R^{9g})(R^{9h}), B⁹—C(O)—B¹⁰—R⁹ⁱ, phenyl, naphthyl (which latter two groups are optionally substituted by one or more substituents selected from OH, halo, C₁₋₄ alkyl and C₁₋₄ alkoxy) and Het¹⁰, and which C₃₋₁₂ cycloalkyl or C₄₋₁₂ cycloalkenyl groups may additionally be substituted by ═O,
(d) OR^{10a},
(e) S(O)ᵤR^{10b},
(f) S(O)₂N(R^{10c})(R^{10d}),
(g) N(R^{10e})S(O)₂R^{10f},
(h) N(R^{10g})(R^{10h}),
(i) B¹¹—C(O)—B¹²—R¹⁰ⁱ,
(j) phenyl (which latter group is optionally substituted by one or more substituents selected from OH, halo, C₁₋₄ alkyl and C₁₋₄ alkoxy) or
(k) Het¹¹;
R^{9a} to R⁹ⁱ and R^{10a} to R¹⁰ⁱ independently represent, at each occurrence,
(a) H,
(b) C₁₋₁₂ alkyl, C₂₋₁₂ alkenyl, C₂₋₁₂ alkynyl, C₃₋₁₂ cycloalkyl, C₄₋₁₂ cycloalkenyl (which latter five groups are optionally substituted by one or more substituents selected from halo, OH, C₁₋₆ alkyl, C₃₋₁₂ cycloalkyl, C₄₋₁₂ cycloalkenyl (which latter two groups are optionally substituted by one or more substituents selected from OH, ═O, halo, C₁₋₄ alkyl and C₁₋₄ alkoxy), C₁₋₆ alkoxy, NH₂, N(H)—C₁₋₆ alkyl, N(C₁₋₆ alkyl)₂, phenyl (which latter group is optionally substituted by one or more substituents selected from OH, halo, C₁₋₄ alkyl and C₁₋₄ alkoxy) and Het¹², and which C₃₋₁₂ cycloalkyl or C₄₋₁₂ cycloalkenyl groups may additionally be substituted by ═O),
(c) phenyl (which latter group is optionally substituted by one or more substituents selected from OH, CN, halo, C₁₋₆ alkyl and C₁₋₆ alkoxy) or
(e) Het¹³,
provided that R^{9b} or R^{10b} does not represent H when t or u, respectively is 1 or 2;
Het¹ to Het¹³ independently represent 4- to 14-membered heterocyclic groups containing one or more heteroatoms selected from oxygen, nitrogen and/or sulfur, which heterocyclic groups may comprise one, two or three rings and may be substituted by one or more substituents selected from
(a) halo,
(b) CN,
(c) C₁₋₁₂ alkyl, C₁₋₁₂ alkenyl, C₁₋₁₂ alkynyl, C₃₋₁₂ cycloalkyl or C₄₋₁₂ cycloalkenyl, which latter five groups are optionally substituted by one or more substituents selected from halo, nitro, CN, C₁₋₆ alkyl, C₁₋₆ alkenyl, C₁₋₆ alkynyl, C₃₋₈ cycloalkyl (which latter three groups are optionally substituted by one or more substituents selected from OH, ═O, halo, C₁₋₄ alkyl and C₁₋₄ alkoxy), OR₁₁ₐ, S(O)ᵥR^{11b}, S(O)₂N(R^{11c})(R^{11d}), N(R^{11e})S(O)₂R^{11f}, N(R^{11g})(R^{11h}), B¹³—C(O)—B¹⁴—R¹¹ⁱ, phenyl, naphthyl (which latter two groups are optionally substituted by one or more substituents selected from OH, halo, C₁₋₄ alkyl and C₁₋₄ alkoxy) and Het^{a}, and which C₃₋₁₂ cycloalkyl or C₄₋₁₂ cycloalkenyl groups may additionally be substituted by ═O,
(d) OR^{12a},
(e) ═O,
(f) S(O)_{w}R^{12b},
(g) S(O)₂N(R^{12c})(R^{12d}),
h) N(R^{12e})S(O)₂R^{12f},
(i) N(R^{12g})(R^{12h}),
(j) B¹⁵—C(O)—B¹⁶—R¹²ⁱ,
(k) phenyl (which latter group is optionally substituted by one or more substituents selected from OH, halo, C₁₋₄ alkyl and C₁₋₄ alkoxy) or
(l) Het^{b};
R^{11a} to R¹¹ⁱ and R^{12a} to R¹²ⁱ independently represent, at each occurrence,
(a) H,
(b) C₁₋₁₂ alkyl, C₂₋₁₂ alkenyl, C₂₋₁₂ alkynyl, C₃₋₁₂ cycloalkyl, C₄₋₁₂ cycloalkenyl (which latter five groups are optionally substituted by one or more substituents selected from halo, OH, C₁₋₆ alkyl, C₃₋₁₂ cycloalkyl, C₄₋₁₂ cycloalkenyl (which latter two groups are optionally substituted by one or more substituents selected from OH, ═O, halo, C₁₋₄ alkyl and C₁₋₄ alkoxy), C₁₋₆ alkoxy, phenyl (which latter group is optionally substituted by one or more substituents selected from OH, halo, C₁₋₄ alkyl and C₁₋₄ alkoxy) and Het, and which C₃₋₁₂ cycloalkyl or C₄₋₁₂ cycloalkenyl groups may additionally be substituted by ═O),
(c) phenyl (which latter group is optionally substituted by one or more substituents selected from OH, halo, C₁₋₄ alkyl and C₁₋₄ alkoxy) or
(e) Het^{d},
provided that R^{11b} or R^{12b} does not represent H when v or w, respectively is 1 or 2;
B¹ to B¹⁶ independently represent a direct bond, O, S, NH or N(R¹³);
n, p, q, r, s, t, u, v and w independently represent 0, 1 or 2;
R¹³ represents
(a) C₁₋₆ alkyl,
(b) phenyl (which latter group is optionally substituted by one or more substituents selected from OH, halo, C₁₋₄ alkyl and C₁₋₄ alkoxy),
(c) C₃₋₇ cycloalkyl (which latter group is are optionally substituted by one or more substituents selected from OH, ═O, halo, C₁₋₄ alkyl and C₁₋₄ alkoxy) or
(e) Het^{e};
Het^{a} to Het^{e} independently represent 5- or 6-membered heterocyclic groups containing one to four heteroatoms selected from oxygen, nitrogen and/or sulfur, which heterocyclic groups may be substituted by one or more substituents selected from halo, ═O and C₁₋₆ alkyl; and
unless otherwise specified (i) alkyl, alkenyl, alkynyl, cycloalkyl, and cycloalkenyl groups, as well as the alkyl part of alkoxy groups, may be substituted by one or more halo atoms, and
(ii) cycloalkyl and cycloalkenyl groups may comprise one or two rings and may additionally be ring-fused to one or two benzene rings.

2. A topical pharmaceutical composition as claimed in claim 1, wherein the compound of formula I is the sole antimicrobial agent in the composition.

3. A combination product for topical administration comprising:
(A) a compound of formula I, as defined in claim 1, or a pharmaceutically-acceptable derivative thereof; and
(B) a conventional antimicrobial agent, or a pharmaceutically-acceptable derivative thereof, and/or
a conventional sterilising agent,
wherein each of components (A) and (B) is formulated in admixture with a pharmaceutically-acceptable adjuvant, diluent or carrier.

4. An intranasal, buccal, intraurethral, intravesical or intravaginal composition comprising a compound of formula I, as defined in claim 1, or a pharmaceutically-acceptable derivative thereof, in admixture with a pharmaceutically acceptable adjuvant, diluent or carrier.

5. A combination product for intranasal, buccal, intraurethral, intravesical or intravaginal administration comprising:
(A) a compound of formula I, as defined in claim 1, or a pharmaceutically-acceptable derivative thereof; and
(B) a conventional antimicrobial agent,
wherein each of components (A) and (B) is formulated in admixture with a pharmaceutically-acceptable adjuvant, diluent or carrier.

6. A topical pharmaceutical composition as claimed in claim 1, wherein the composition is in the form of a cream, ointment, lotion, spray, gel or sterile aqueous solution or suspension.

7. A topical pharmaceutical composition as claimed in claim 6, wherein the composition comprises the compound of formula I and one or more components selected from: water; aqueous buffer solutions; non-aqueous solvents; and oils.

8. A topical pharmaceutical composition as claimed in claim 6, wherein the composition comprises a compound of formula I, water and one or more components selected from: a solubilising agent or solvent; a thickening agent; a gelling agent; a preservative; and pH buffering agent(s).

9. A topical pharmaceutical composition as claimed in claim 6, wherein the composition comprises a compound of formula I and:
(a) water;
(b) one or more polar, non-aqueous solvents;
(c) a preservative;
(d) a thickening agent; and, optionally
(e) pH buffering agent(s).

10. A topical pharmaceutical composition as claimed in claim 9, wherein the composition comprises 0.5 to 3% by weight of the compound of formula I.

11. A topical pharmaceutical composition as claimed in claim 9, wherein the one or more polar, non-aqueous solvents are one or more of ethanol, propylene glycol and glycerol.

12. A topical pharmaceutical composition as claimed in claim 9, wherein the preservative is benzyl alcohol.

13. A topical pharmaceutical composition as claimed in claim 9, wherein the thickening agent is hydroxyethylcellulose.

14. A topical pharmaceutical composition as claimed in claim 9, wherein:
(i) water is present at from 55 to 75% by weight;
(ii) the one or more polar, non-aqueous solvents are together present at from 15 to 40% by weight;
(iii) glycerol, if used, is present at from 5 to 25% by weight;
(iv) ethanol, if used, is present at from 3 to 10% by weight;
(v) propylene glycol, if used, is present at from 2 to 15% by weight;
(vi) the preservative is present at from 0.1 to 3% (e.g. about 1%) by weight; and
(vii) the thickening agent is present at from 1 to 5% (e.g. about 2% by weight).

15. A topical pharmaceutical composition as claimed in claim 9, wherein the one or more polar, non-aqueous solvents comprises a mixture of ethanol, propylene glycol and glycerol.

16. A topical pharmaceutical composition as claimed in claim 1, wherein the compound of formula I is a compound of formula Ib, 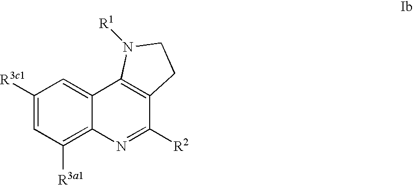
wherein:
R¹ represents
(a) C₁₋₅ alkyl, which latter group is optionally substituted by C₃₋₅ cycloalkyl, phenyl (which latter group is optionally substituted by one or more substituents selected from halo, methyl and methoxy), phenoxy, benzodioxanyl or benzodioxolyl,
(b) C₃₋₆ cycloalkyl, which latter group is optionally fused to a benzene ring,
(c) phenyl, which latter group is optionally substituted by one or more substituents selected from halo, C₁₋₄ alkyl, OH, C₁₋₄ alkoxy (which latter group is optionally substituted by N(CH₃)₂), phenoxy (which latter group is optionally substituted by one or more substituents selected from methoxy and halo), piperidin-1-yl, pyridyloxy and piperazinyl (which latter group is optionally substituted by methyl),
(d) pyridyl, which latter group is optionally substituted by methoxy or phenoxy, or
(e) piperidinyl, which latter group is optionally substituted by C₁₋₂ alkyl (which latter group is optionally substituted by phenyl);
R² represents C₁₋₃ alkyl optionally substituted by one or more halo substituents;
R^{3a1} represents H and R^{3e1} represents phenoxy,
or, when R¹ represents
C₁₋₂ alkyl substituted by optionally substituted phenyl,
C₅₋₆ cycloalkyl fused to a benzene ring, or
phenyl substituted by phenoxy or piperidin-1-yl,
then R^{3a1} can additionally represent methoxy, or phenoxy and R^{3c1} can additionally represent H, piperidin-1-yl, methoxy, trifluoromethoxy or ethoxy,
provided that R^{3a1} and R^{3e1} do not both represent phenoxy.

17. A topical pharmaceutical composition as claimed in claim 1, wherein the compound of formula I is any compound selected from:
(1) 6,8-dimethoxy-4-methyl-1-(3-phenoxyphenyl)-2,3-dihydro-1H-pyrrolo[3,2-c]-quinoline;
(2) 6,8-dimethoxy-4-methyl-1-(2-phenoxyethyl)-2,3-dihydro-1H-pyrrolo[3,2-c]-quinoline;
(3) 1-cyclopropyl-6,8-dimethoxy-4-methyl-2,3-dihydro-1H-pyrrolo[3,2-c]-quinoline;
(4) 8-methoxy-4-methyl-1-(4-phenoxyphenyl)-2,3-dihydro-1H-pyrrolo[3,2-c]-quinoline;
(5) (2-[4-(8-methoxy-4-methyl-2,3-dihydro-1H-pyrrolo[3,2-c]quinolin-1-yl)-phenyoxy]ethyl}dimethylamine;
(6) 8-methoxy-4-methyl-1-[4-(pyridin-3-yloxy)phenyl]-2,3-dihydro-1H-pyrrolo[3,2-c]quinoline;
(7) 4-methyl-8-phenoxy-1-phenyl-2,3-dihydro-1H-pyrrolo[3,2-c]quinoline;
(8) 1-benzyl-4-methyl-8-phenoxy-2,3-dihydro-1H-pyrrolo[3,2-c]quinoline
(9) 1-(indan-2-yl)-4-methyl-8-phenoxy-2,3-dihydro-1H-pyrrolo[3,2-c]quinoline
(10) 4-methyl-6-phenoxy-1-phenyl-2,3-dihydro-1H-pyrrolo[3,2-c]quinoline;
(11) 1-benzyl-4-methyl-6-phenoxy-2,3-dihydro-1H-pyrrolo[3,2-c]quinoline;
(12) 1-(indan-2-yl)-4-methyl-6-phenoxy-2,3-dihydro-1H-pyrrolo[3,2-c]quinoline;
(13) 4-methyl-1-(2-phenylethyl)-8-phenoxy-2,3-dihydro-1H-pyrrolo[3,2-c]-quinoline;
(14) 8-methoxy-4-methyl-1-(2-phenylethyl)-2,3-dihydro-1H-pyrrolo[3,2-c]-quinolin-6-ol;
(15) 1-(1-benzyl-piperidin-4-yl)-4-methyl-8-phenoxy-2,3-dihydro-1H-pyrrolo[3,2-c]quinoline;
(16) 1-(indan-1-yl)-4-methyl-8-phenoxy-2,3-dihydro-1H-pyrrolo[3,2-c]quinoline;
(17) 1-(benzodioxan-2-ylmethyl)-4-methyl-8-phenoxy-2,3-dihydro-1H-pyrrolo[3,2-c]quinoline;
(18) 4-methyl-8-phenoxy-1-(1,2,3,4-tetrahydronaphthalen-1-yl)-2,3-dihydro-1H-pyrrolo[3,2-c]quinoline;
(19) 1-cyclohexyl-4-methyl-8-phenoxy-2,3-dihydro-1H-pyrrolo[3,2-c]quinoline;
(20) 8-ethoxy-4-methyl-1-(4-phenoxyphenyl)-2,3-dihydro-1H-pyrrolo[3,2-c]-quinoline;
(21) 1-(4-methoxyphenyl)-4-methyl-8-phenoxy-2,3-dihydro-1H-pyrrolo[3,2-c]-quinoline;
(22) 4-methyl-1-(4-phenoxyphenyl)-2,3-dihydro-1H-pyrrolo[3,2-c]quinoline;
(23) 4-methyl-1-(2-methylphenyl)methyl-8-phenoxy-2,3-dihydro-1H-pyrrolo[3,2-c]quinoline;
(24) 4-methyl-8-phenoxy-1-(4-iso-propylphenyl)-2,3-dihydro-1H-pyrrolo[3,2-c]-quinoline;
(25) 4-methyl-8-phenoxy-1-(1-phenylethyl)-2,3-dihydro-1H-pyrrolo[3,2-c]-quinoline;
(26) 8-methoxy-4-methyl-1-(2-phenylethyl)-2,3-dihydro-1H-pyrrolo[3,2-c]-quinoline;
(27) 6,8-dimethoxy-1-(4-hydroxyphenyl)-4-methyl-2,3-dihydro-1H-pyrrolo[3,2-c]quinoline;
(28) 6,8-dimethoxy-1-(3-hydroxyphenyl)-4-methyl-2,3-dihydro-1H-pyrrolo[3,2-c]quinoline;
(29) 6,8-dimethoxy-1-(3-hydroxy-5-methylphenyl)-4-methyl-2,3-dihydro-1H-pyrrolo[3,2-c]quinoline;
(30) 8-methoxy-1-(4-methoxyphenyl)-4-methyl-2,3-dihydro-1H-pyrrolo[3,2-c]-quinoline;
(31) 8-trifluoromethoxy-1-(4-phenoxyphenyl)-4-methyl-2,3-dihydro-1H-pyrrolo[3,2-c]quinoline;
(32) 6,8-dimethoxy-4-methyl-1-[4-(pyridin-3-yloxy)phenyl]-2,3-dihydro-1H-pyrrolo[3,2-c]quinoline;
(33) 1-benzyl-6,8-dimethoxy-4-methyl-2,3-dihydro-1H-pyrrolo[3,2-c]quinoline;
(34) 6,8-dimethoxy-4-methyl-1-(2-phenylethyl)-2,3-dihydro-1H-pyrrolo[3,2-c]quinoline;
(35) 4-methyl-1-(2-phenylethyl)-8-trifluoromethoxy-2,3-dihydro-1H-pyrrolo[3,2-c]quinoline;
(36) 6,8-dimethoxy-1-(indan-1-yl)-4-methyl-2,3-dihydro-1H-pyrrolo[3,2-c]-quinoline;
(37) 6,8-dimethoxy-4-methyl-1-[(6-phenoxy)pyridin-3-yl]-2,3-dihydro-1H-pyrrolo[3,2-c]quinoline;
(38) 6,8-dimethoxy-1-[(6-methoxy)pyridin-3-yl]-4-methyl-2,3-dihydro-1H-pyrrolo[3,2-c]quinoline;
(39) 1-(benzodioxol-5-ylmethyl)-6,8-dimethoxy-4-methyl-2,3-dihydro-1H-pyrrolo[3,2-c]quinoline;
(40) 6,8-dimethoxy-4-methyl-1-(3-methylbutyl)-2,3-dihydro-1H-pyrrolo[3,2-c]-quinoline;
(41) 1-cyclopropylmethyl-6,8-dimethoxy-4-methyl-2,3-dihydro-1H-pyrrolo[3,2-c]quinoline;
(42) 4-methyl-8-(morpholin-4-yl)-1-(4-phenoxyphenyl)-2,3-dihydro-1H-pyrrolo[3,2-c]quinoline;
(43) 8-methoxy-4-methyl-1-(1,2,3,4-tetrahydronaphthalen-1-yl)-2,3-dihydro-1H-pyrrolo[3,2-c]quinoline;
(44) 4-methyl-1-(2-phenylethyl)-2,3-dihydro-1H-pyrrolo[3,2-c]quinoline;
(45) 4,6-dimethyl-1-(2-methylphenyl)-2,3-dihydro-1H-pyrrolo[3,2-c]quinoline;
(46) 4,6-dimethyl-1-(2-phenylethyl)-2,3-dihydro-1H-pyrrolo[3,2-c]quinoline;
(47) 4-methyl-8-(piperidin-1-yl)-1-[4-(piperidin-1-yl)phenyl]-2,3-pyrrolo[3,2-c]quinoline;
(48) 4-methyl-8-(piperidin-1-yl)-1-(3-phenoxyphenyl)-2,3-dihydro-1H-pyrrolo[3,2-c]quinoline;
(49) 1-{4-[2-(N,N-dimethylamino)ethoxy]phenyl}-4-methyl-8-phenoxy-2,3-dihydro-1H-pyrrolo[3,2-c]quinoline;
(50) 1-[4-(4-fluorophenoxy)phenyl]-8-methoxy-4-methyl-2,3-dihydro-1H-pyrrolo[3,2-c]quinoline;
(51) 1-(benzodioxan-2-ylmethyl)-8-methoxy-4-methyl-2,3-dihydro-1H-pyrrolo[3,2-c]quinoline ;
(52) 1-cyclohexyl-8-methoxy-4-methyl-2,3-dihydro-1H-pyrrolo[3,2-c]quinoline;
(53) 8-methoxy-4-methyl-1-phenyl-2,3-dihydro-1H-pyrrolo[3,2-c]quinoline;
(54) 4-methyl-8-phenoxy-1-[4-(3-pyridyl)phenyl]-2,3-dihydro-1H-pyrrolo[3,2-c]-quinoline;
(55) 4-methyl-8-phenoxy-1-[2-(3-pyridyl)ethyl]-2,3-dihydro-1H-pyrrolo[3,2-c]-quinoline;
(56) 4-methyl-8-phenoxy-1-(2-pyridylmethyl)-2,3-dihydro-1H-pyrrolo[3,2-c]-quinoline;
(57) 4-methyl-1-(5-methylpyrazin-2-ylmethyl)-8-phenoxy-2,3-dihydro-1H-pyrrolo[3,2-c]quinoline;
(58) 8-chloro-4-methyl-1-(2-phenylethyl)-2,3-dihydro-1H-pyrrolo[3,2-c]-quinoline;
(59) methyl 4-methyl-1-(2-phenylethyl)-2,3-dihydro-1H-pyrrolo[3,2-c]quinoline-8-carboxylate;
(60) 4-methyl-8-(morpholin-1-yl)-1-(2-phenylethyl)-2,3-dihydro-1H-pyrrolo[3,2-c]quinoline;
(61) ethyl [4-methyl-1-(2-phenylethyl)-2,3-dihydro-1H-pyrrolo[3,2-c]quinoline-8-yl]acetate;
(62) 1-[3-(4-methyl-8-phenoxy-2,3-dihydro-1H-pyrrolo[3,2-c]quinolin-1-yl)propyl]-pyrrolidin-2-one;
(63) 4-methyl-8-phenoxy-142-(2-pyridyl)ethyl]-2,3-dihydro-1H-pyrrolo[3,2-c]quinoline;
(64) ethyl 3-(8-methoxy-4-methyl-2,3-dihydro-1H-pyrrolo[3,2-c]quinoline-1-yl)propionate;
(65) ethyl 4-(4-methyl-8-phenoxy-2,3-dihydro-1H-pyrrolo[3,2-c]quinoline-1-yl)butanoate;
(66) methyl 4-(4-methyl-8-phenoxy-2,3-dihydro-1H-pyrrolo[3,2-c]quinoline-1-yl)butanoate;
(67) ethyl (4-methyl-8-phenoxy-2,3-dihydro-1H-pyrrolo[3,2-c]quinoline-1-yl)acetate;
(68) 4-methyl-1-(1-methylpiperidin-4-yl)-8-phenoxy-2,3-dihydro-1H-pyrrolo[3,2-c]quinoline;
(69) 1-(1-benzylpyrrolidin-3-yl)-8-methoxy-4-methyl-2,3-dihydro-1H-pyrrolo[3,2-c]quinoline;
(70) methyl 3-(4-methyl-8-phenoxy-2,3-dihydro-1H-pyrrolo[3,2-c]quinoline-1-yl)propionate;
(71) 1((S)-indan-1-yl)-4-methyl-8-phenoxy-2,3-dihydro-1H-pyrrolo[3,2-c]-quinoline;
(72) 1-((R)-indan-1-yl)-4-methyl-8-phenoxy-2,3-dihydro-1H-pyrrolo[3,2-c]-quinoline;
(73) 1-(3-methoxypropyl)-4-methyl-8-phenoxy-2,3-dihydro-1H-pyrrolo[3,2-4-quinoline;
(74) 4-methyl-8-phenoxy-1-(tetrahydrofuran-2-ylmethyl)-2,3-dihydro-1H-pyrrolo[3,2-c]quinoline;
(75) 1-[2-(4-chlorophenyl)ethyl]-4-methyl-8-phenoxy-2,3-dihydro-1H-pyrrolo[3,2-c]quinoline;
(76) 1-[2-(4-methoxyphenyl)ethyl]-4-methyl-8-phenoxy-2,3-dihydro-1H-pyrrolo[3,2-c]quinoline;
(77) 4-methyl-8-phenoxy-1-(2-phenylpropyl)-2,3-dihydro-1H-pyrrolo[3,2-c]-quinoline;
(78) 8-cyano-4-methyl-1-(2-phenylethyl)-2,3-dihydro-1H-pyrrolo[3,2-c]quinoline;
(79) 8-hydroxy-4-methyl-1-(2-phenylethyl)-2,3-dihydro-1H-pyrrolo[3,2-c]-quinoline;
(80) 8-phenoxy-1-(2-phenylethyl)-2,3-dihydro-1H-pyrrolo[3,2-c]quinoline;
(81) 6,8-dimethoxy-1-(4-hydroxyphenyl)-4-methylpyrrolo[3,2-c]quinoline;
(82) 8-methoxy-4-methyl-1-[4-(4-methylpiperazin-1-yl)-3-fluorophenyl]-2,3-dihydro-1H-pyrrolo[3,2-c]quinoline;
(83) 4-methyl-8-phenylamino-1-(2-phenylethyl)-2,3-dihydro-1H-pyrrolo[3,2-c]-quinoline;
(84) [4-methyl-1-(2-phenylethyl)-2,3-dihydro-1H-pyrrolo[2,3-c]quinoline-8-oyl]-piperidine, (85) 6,8-dimethoxy-1-(4-iso-propylphenyl)-4-methyl-2,3-dihydro-1H-pyrrolo[3,2-c]quinoline;
(86) 6-methoxy-1-(4-phenoxyphenyl)-4-methyl-2,3-dihydro-1H-pyrrolo[3,2-c]quinoline;
(87) 6-methoxy-1-(4-iso-propylphenyl)-4-methyl-2,3-dihydro-1H-pyrrolo[3,2-c]quinoline;
(88) 6,8-dimethoxy-1-(4-phenoxyphenyl)-4-methyl-2,3-dihydro-1H-pyrrolo[3,2-c]quinoline;
(89) 4-methyl-8-phenoxy-1-(4-phenoxyphenyl)-2,3-dihydro-1H-pyrrolo[3,2-c]quinoline;
(90) 1-(4-iso-propylphenyl)-6-phenoxy-4-methyl-2,3-dihydro-1H-pyrrolo[3,2-c]quinoline; and
(91) 4,6-dimethyl-1-(4-methylphenyl)-2,3-dihydro-1H-pyrrolo[3,2-c]quinoline,
and pharmaceutically-acceptable salts and/or solvates thereof.

18. A topical pharmaceutical composition as claimed in claim 1 or claim 16, wherein: R¹ represents cyclohexyl or 2-phenylethyl; R² represents methyl; R^{3a} represents H; and/or R^{3b} represents phenoxy.

19. A topical pharmaceutical composition as claimed in claim 1 or claim 16, wherein the compound of formula I is:
1-cyclohexyl-4-methyl-8-phenoxy-2,3-dihydro-1H-pyrrolo[3,2-c]quinoline; or
4-methyl-1-(2-phenylethyl)-8-phenoxy-2,3-dihydro-1H-pyrrolo[3,2-c]quinoline,
or a pharmaceutically-acceptable salt and/or solvate thereof.

20. A method of preparing a topical pharmaceutical composition according to claim 1, which method comprises mixing together the components of the composition.

21. A method for treating: acne vulgaris; acne rosacea; rosacea; erysipelas; erythrasma; ecthyma; ecthyma gangrenosum; impetigo; paronychia; cellulitis; folliculitis; furunculosis; carbunculosis; staphylococcal scalded skin syndrome; surgical scarlet fever; streptococcal peri-anal disease; streptococcal toxic shock syndr ome; pitted keratolysis; trichomycosis axillaris; pyoderma; external canal ear infections; green nail syndrome; spirochetes; necrotizing fasciitis; Mycobacterial skin infections; atopic eczma with staphylococcal carriage; or infected eczma, burns, abrasions or skin wounds, said method comprising administering to a patient in need thereof an effective amount of a topical pharmaceutical composition as defined in claim 1 or claim 16.

22. A topical pharmaceutical composition as defined in claim 1 or claim 16, for use in the treatment of: acne vulgaris; acne rosacea; rosacea; erysipelas; erythrasma; ecthyma; ecthyma gangrenosum; impetigo; paronychia; cellulitis; folliculitis; furunculosis; carbunculosis; staphylococcal scalded skin syndrome; surgical scarlet fever; streptococcal peri-anal disease; streptococcal toxic shock syndr ome; pitted keratolysis; trichomycosis axillaris; pyoderma; external canal ear infections; green nail syndrome; spirochetes; necrotizing fasciitis; Mycobacterial skin infections; atopic eczma with staphylococcal carriage; or infected eczma, burns, abrasions or skin wounds.

23. A method for effecting clearance of Staphylococci, Propionibacteria or fungi from the skin or membranes of a patient in need of such clearance, said method comprising administering to a patient in need thereof an effective amount of a topical pharmaceutical composition as defined in claim 1 or claim 16.

24. A topical pharmaceutical composition as defined in claim 1 or claim 16, for use in the clearance of Staphylococci, Propionibacteria or fungi from the skin or membranes of a patient in need of such clearance.

25. A method of killing clinically latent microorganisms in a mammal infected with such latent microorganisms, the method comprising administering to said mammal a microbicidally effective amount of a topical pharmaceutical composition as defined in claim 1 and claim 16.

26. A topical pharmaceutical composition as defined in claim 1 and claim 16, for use in the killing of clinically latent microorganisms in a mammal infected with such latent microorganisms.

27. A method of treating a topical protozoal disease, said method comprising administering to a patient in need thereof an effective amount of a topical pharmaceutical composition as defined in claim 1 or claim 16.

28. A topical pharmaceutical composition as defined in claim 1 for use in the treatment of a topical protozoal disease.

29. A method as claimed in claim 27, or a topical pharmaceutical composition for use as claimed in claim 28, wherein the topical protozoal disease is Leishmaniasis or an infection with Trichomonas vaginalis.
